SEQUENCE LISTING (61 sequences)

SEQ ID NO: 1 - protein, 265 aa, Mouse
MALPVTALLLPLALLLHAARPELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGGGGSGGGGSGGGGSQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 2 - protein, 270 aa, Mouse
MALPVTALLLPLALLLHAARPDIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWMGHFSPGNTDIKYNDKFKGRVTITADKSTSTAYMELSSLRSEDTAVYYCKTSTFFFDYWGQGTTVTVSS

SEQ ID NO: 3 - protein, 270 aa, Mouse
MALPVTALLLPLALLLHAARPDIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSS

SEQ ID NO: 4 - protein, 270 aa, Mouse
MALPVTALLLPLALLLHAARPELVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSS

SEQ ID NO: 5 - protein, 270 aa, Mouse
MALPVTALLLPLALLLHAARPDIVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWMGHFSPGNTDIKYNDKFKGRVTITADKSTSTAYMELSSLRSEDTAVYYCKTSTFFFDYWGQGTTVTVSS

SEQ ID NO: 6 - protein, 270 aa, Mouse
MALPVTALLLPLALLLHAARPDIVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSS

SEQ ID NO: 7 - protein, 270 aa, Mouse
MALPVTALLLPLALLLHAARPELVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSS

SEQ ID NO: 8 - protein, 21 aa, Mouse
MALPVTALLLPLALLLHAARP

SEQ ID NO: 9 - protein, 15 aa, Mouse
GGGGSGGGGSGGGGS

SEQ ID NO: 10 - protein, 17 aa, Mouse
KSSQSLLNSGDQKNYLT

SEQ ID NO: 11 - protein, 7 aa, Mouse
WASTRES

SEQ ID NO: 12 - protein, 9 aa, Mouse
QNDYSYPLT

SEQ ID NO: 13 - protein, 5 aa, Mouse
DHAIH

SEQ ID NO: 14 - protein, 17 aa, Mouse
HFSPGNTDIKYNDKFKG

SEQ ID NO: 15 - protein, 7 aa, Mouse
STFFFDY

SEQ ID NO: 16 - protein, 17 aa, Mouse
KSSQSLLNSGDQKNYLT

SEQ ID NO: 17 - protein, 7 aa, Mouse
WASTRES

SEQ ID NO: 18 - protein, 9 aa, Mouse
QNDYSYPLT

SEQ ID NO: 19 - protein, 5 aa, Mouse
DHAIH

SEQ ID NO: 20 - protein, 17 aa, Mouse
HFSPGNTDIKYNDKFKG

SEQ ID NO: 21 - protein, 7 aa, Mouse
STFFFDY

SEQ ID NO: 22 - protein, 17 aa, Mouse
KSSQSLLNSGDQKNYLT

SEQ ID NO: 23 - protein, 7 aa, Mouse
WASTRES

SEQ ID NO: 24 - protein, 9 aa, Mouse
QNDYSYPLT

SEQ ID NO: 25 - protein, 5 aa, Mouse
DHAIH

SEQ ID NO: 26 - protein, 17 aa, Mouse
HFSPGNTDIKYNDKFKG

SEQ ID NO: 27 - protein, 7 aa, Mouse
STFFFDY

SEQ ID NO: 28 - protein, 17 aa, Mouse
KSSQSLLNSGDQKNYLT

SEQ ID NO: 29 - protein, 7 aa, Mouse
WASTRES

SEQ ID NO: 30 - protein, 9 aa, Mouse
QNDYSYPLT

SEQ ID NO: 31 - protein, 5 aa, Mouse
DHAIH

SEQ ID NO: 32 - protein, 17 aa, Mouse
HFSPGNTDIKYNDKFKG

SEQ ID NO: 33 - protein, 7 aa, Mouse
STFFFDY

SEQ ID NO: 34 - protein, 17 aa, Mouse
KSSQSLLNSGDQKNYLT

SEQ ID NO: 35 - protein, 7 aa, Mouse
WASTRES

SEQ ID NO: 36 - protein, 9 aa, Mouse
QNDYSYPLT

SEQ ID NO: 37 - protein, 5 aa, Mouse
DHAIH

SEQ ID NO: 38 - protein, 17 aa, Mouse
HFSPGNTDIKYNDKFKG

SEQ ID NO: 39 - protein, 7 aa, Mouse
STFFFDY

SEQ ID NO: 40 - protein, 17 aa, Mouse
KSSQSLLNSGDQKNYLT

SEQ ID NO: 41 - protein, 7 aa, Mouse
WASTRES

SEQ ID NO: 42 - protein, 9 aa, Mouse
QNDYSYPLT

SEQ ID NO: 43 - protein, 5 aa, Mouse
DHAIH

SEQ ID NO: 44 - protein, 17 aa, Mouse
HFSPGNTDIKYNDKFKG

SEQ ID NO: 45 - protein, 7 aa, Mouse
STFFFDY

SEQ ID NO: 46 - protein, 113 aa, Mouse
DIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIK

SEQ ID NO: 47 - protein, 116 aa, Mouse
QVQLVQSGAEVKKPGSSVKVSCKASGYTFTDHAIHWVRQAPGQGLEWMGHFSPGNTDIKYNDKFKGRVTITADKSTSTAYMELSSLRSEDTAVYYCKTSTFFFDYWGQGTTVTVSS

SEQ ID NO: 48 - protein, 113 aa, Mouse; identical to SEQ ID NO: 46

SEQ ID NO: 49 - protein, 116 aa, Mouse
QVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSS

SEQ ID NO: 50 - protein, 113 aa, Mouse
ELVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIK

SEQ ID NO: 51 - protein, 116 aa, Mouse; identical to SEQ ID NO: 49

SEQ ID NO: 52 - protein, 113 aa, Mouse
DIVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIK

SEQ ID NO: 53 - protein, 116 aa, Mouse; identical to SEQ ID NO: 47

SEQ ID NO: 54 - protein, 113 aa, Mouse; identical to SEQ ID NO: 52

SEQ ID NO: 55 - protein, 116 aa, Mouse; identical to SEQ ID NO: 49

SEQ ID NO: 56 - protein, 113 aa, Mouse
ELVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIK

SEQ ID NO: 57 - protein, 116 aa, Mouse; identical to SEQ ID NO: 49

SEQ ID NO: 58 - protein, 509 aa, Mouse
MALPVTALLLPLALLLHAARPDIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWMGHFSPGNTDIKYNDKFKGRVTITADKSTSTAYMELSSLRSEDTAVYYCKTSTFFFDYWGQGTTVTVSSRTFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 509 aa, Mouse
MALPVTALLLPLALLLHAARPDIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSSRTFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 509 aa, Mouse
MALPVTALLLPLALLLHAARPDIVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWMGHFSPGNTDIKYNDKFKGRVTITADKSTSTAYMELSSLRSEDTAVYYCKTSTFFFDYWGQGTTVTVSSRTFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 509 aa, Mouse
MALPVTALLLPLALLLHAARPDIVMTQSPDSLAVSLGERATINCKSSQSLLNSGDQKNYLTWYQQKPGQPPKLLIFWASTRESGVPDRFSGSGSGTDFTLTISSLQAEDVAVYYCQNDYSYPLTFGQGTKLEIKGGGGSGGGGSGGGGSGGGGSQVQLVQSGAEVKKPGSSVKVSCKASGYTFTDHAIHWVRQAPGQGLEWIGHFSPGNTDIKYNDKFKGRATLTVDRSTSTAYMELSSLRSEDTAVYFCKTSTFFFDYWGQGTTVTVSSRTFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0374151 A1
(43) Pub. Date: Nov. 23, 2023

(54) ANTI-TN-MUC1 CHIMERIC ANTIGEN RECEPTORS

(71) Applicant: GLAXOSMITHKLINE INTELLECTUAL PROPERTY DEVELOPMENT LIMITED, Brentford (GB)

(72) Inventors: Tejinder Kaur BHINDER, Stevenage (GB); Nicole CHRIST, STEVENAGE (GB); Sergey LEKOMTSEV, Stevenage (GB); ALAN Peter LEWIS, Stevenage (GB)

(21) Appl. No.: 17/913,072

(22) PCT Filed: Mar. 24, 2021
(86) PCT No.: PCT/IB2021/052447

§ 371 Date: Sep. 20, 2022

(30) Foreign Application Priority Data
Mar. 26, 2020 (GB) 2004371.7

(51) Int. Cl. C07K 16/30; C07K 14/725; C12N 5/0783; C07K 16/28; A61K 39/00; A61P 35/00

(52) U.S. Cl. CPC C07K 16/3092; C07K 14/7051; C12N 5/0636; C07K 16/2878; A61K 39/4611; A61K 39/46447; A61P 35/00; C07K 2317/24; C07K 2319/03; C07K 2319/33; C07K 2317/92; C07K 2317/622; A61K 2239/13; A61K 2239/22

(57) ABSTRACT

The present invention relates to improved compositions and methods for treating diseases, such as cancers that express aberrantly glycosylated MUC1 proteins, by providing a cell immunotherapy, wherein the cell immunotherapy is an immunomodulatory cell expressing a chimeric antigen receptor (CAR) that binds aberrantly glycosylate MUC1 proteins. The invention further relates to polynucleotides, expression vectors, and immunomodulatory cells comprising the immunotherapy, as well as related methods.

### CROSS REFERENCE TO RELATED APPLICATION

This application claims priority to GB Patent Application No. 2004371.7, filed 26 Mar. 2020, the contents of which are incorporated by reference herein.

### SEQUENCE LISTING SUBMITTED ELECTRONICALLY

The instant application contains a Sequence Listing which has been submitted electronically in ASCII format and is hereby incorporated by reference in its entirety. Said ASCII copy, created on 2 Mar. 2021, is named “PB66802 Sequence Listing” and is 107 KB in size.

### FIELD OF THE INVENTION

The present invention relates to chimeric antigen receptor (CAR) molecules, polypeptides comprising such CAR molecules, polynucleotides encoding such CAR molecules, and vectors comprising such polynucleotides. The present invention also relates to immune effector cells comprising such CAR molecules, polypeptides, polynucleotides, and vectors. The present invention also relates to pharmaceutical compositions comprising such immune effector cells. The present invention also relates to the use of such CAR molecules, polypeptides, polynucleotides, vectors, immune effector cells, and pharmaceutical compositions for the treatment of cancers which comprise cells expressing aberrantly glycosylated MUC1 proteins.

### BACKGROUND TO THE INVENTION

Adoptive T cell therapies are transformative medicines with curative potential for cancer patients. To engineer these potent autologous cell therapies for patients, peripheral blood is used to obtain T cells which are then genetically modified. Introducing a chimeric antigen receptor (CAR) to these cells enables them to specifically bind to an antigen of choice. These modified cells are multiplied ex vivo and reinfused into the patient with the objective of trafficking to and subsequently killing cancer cells expressing the matching antigens (Yeku et al., Am Soc Clin Oncol Educ Book. 2017; 37: 193-204; McBride et al., Wiley Interdiscip Rev Nanomed Nanobiotechnol. 2019; 11(5): e1557).

CARs are synthetic antigen receptors that reprogram T cell specificity, function and persistence. CARs are composed of the antigen specific region of an antibody single chain variable fragment (scFv) fused to the T cells activating domain, e.g., zeta chain of the CD3 complex, and a co-stimulatory domain, e.g., CD28 or 4-1BB. This configuration promotes antigen specific activation and enhances proliferation and antiapoptotic functions of human primary T cells.

CAR-T cells have demonstrated remarkable efficacy against a range of liquid tumour B-cell malignancies; with results of early clinical trials suggesting activity in multiple myeloma (Sadelain et al., Nature. 2017; 545(7655): 423-31; June et al., N Engl J Med. 2018; 379(1): 64-73; Brudno et al., Nat Rev Clin Oncol. 2017; 15(1): 31-46). The 2017 FDA approval of CD19 CAR-T's for lymphoma and leukaemia has reinvigorated focused efforts in developing CAR-T cells for solid tumours. However, CAR-T cell therapies have demonstrated limited efficacy in solid tumours to date.

To generate a safe and efficacious T cell therapy against solid cancers, the T cells must retain a high and efficacious killing potential throughout manufacturing, be capable of trafficking to the tumour, and overcome the immunosuppressive tumour microenvironment (TME). One of the major success factors is the selection of the antigen target. The antigen must be expressed in sufficient amounts on the cancer cell surface, while normal tissue expression remains low to ensure a low cross-reactivity to healthy cells (both off- and on-target effect). CAR immunotherapy in solid tumours remains challenging, largely due to the lack of appropriate surface antigens whose expression is confined to malignant tissue. Off-tumour expression of the antigen target has potential to cause on-target toxicity with varying degrees of severity depending on the affected organ tissue (Watanabe et al., Front Immunol. 2018; 9: 2486; Park et al, Sci Rep. 2017; 7(1): 14366).

Cell surface associated mucin 1 (MUC1), is a large protein with tandem repeated sequences carrying 0-glycans and is expressed on the apical surface of normal cells. The aberrantly glycosylated form of MUC1 with truncated glycoforms in cancer (FIG. 1) is overexpressed in most adenocarcinomas. The most prevalent aberrant glycoforms found in cancer are the Tn and sialyl-Tn (sTn) glycoforms. Several mechanisms might result in accumulation of Tn glycoforms, including loss of T synthase activity due to mutations or epigenetic silencing of COSMC and ectopic expression of GalNAc-Ts23. In healthy tissues, the Tn antigen is rarely expressed, and humans have natural anti-Tn IgM antibodies. Furthermore, unlike normal epithelium where MUC1 is confined to the apical surface, MUC-1 becomes aberrantly over-expressed on tumor cell membranes (FIG. 1). However, despite data showing that aberrant glycosylation is predominantly present on the cellular surface of mutated cancer cells, it cannot be ruled out completely that low levels of aberrantly glycosylated MUC1 are present on healthy or inflamed cells that express MUC1 protein (Cascio et al., Biomolecules. 2016; 6(4): E39). High-affinity glycopeptide-specific antibodies have recently been developed to target TnMUC1. The mouse 5E5 mAb can lyse breast cancer cells via complement mediated and antibody-dependent cellular cytotoxicity. TnMUC1 specific CAR-T cells, which comprise the variable domains of the 5E5 mAb, can eliminate pancreatic and leukemia in xenograft models and similar to the original antibody, display cancer-specificity with negligible reactivity against normal tissues.

The present inventors have identified the Tn-mucin 1 (TnMUC1) antigen as a potential target for CAR-T development and have engineered a second-generation CAR specifically targeting the Tn-MUC1 antigen. The TnMUC1 targeting CAR-T demonstrates high specificity to TnMUC1 antigen, low level of basal activation and high potency in killing of TnMUC1 positive tumour cells.

### SUMMARY OF THE INVENTION

The present invention provides, in a first aspect, a chimeric antigen receptor (CAR) comprising:
a) an extracellular domain which comprises a humanised antibody or antigen binding domain thereof that binds one or more epitopes on an aberrantly glycosylated MUC1 protein, wherein the antibody or antigen binding domain thereof comprises:
   a CDRL1 sequence at least 90% identical to SEQ ID NO: 28;
   a CDRL2 sequence at least 90% identical to SEQ ID NO: 29;
   a CDRL3 sequence at least 90% identical to SEQ ID NO: 30;
   a CDRH1 sequence at least 90% identical to SEQ ID NO: 31;
   a CDRH2 sequence at least 90% identical to SEQ ID NO: 32; and
   a CDRH3 sequence at least 90% identical to SEQ ID NO: 33,
and wherein the antibody or antigen binding fragment thereof binds said epitope with a faster dissociation rate constant (k_{d}) as compared to a non-humanised version of said antibody or antigen binding domain thereof, and
b) a transmembrane domain;
c) one or more costimulatory domains; and
d) one or more intracellular signalling domains

The present invention also provides, in a second aspect, a polypeptide comprising the amino acid sequence of a CAR according to the first aspect of the invention.

The present invention also provides, in a third aspect, a polynucleotide encoding a CAR according to the first aspect of the invention.

The present invention also provides, in a fourth aspect, a vector comprising a polynucleotide according to the third aspect of the invention.

The present invention also provides, in a fifth aspect, a vector producer cell comprising a polynucleotide according to the third aspect of the invention and/or the vector according to a fourth aspect of the invention.

The present invention also provides, in a sixth aspect, an immune effector cell comprising a CAR according to the first aspect of the invention, a polypeptide according to the second aspect of the invention, a polynucleotide according to the third aspect of the invention, or a vector according to the fourth aspect of the invention.

The present invention also provides, in a seventh aspect, a pharmaceutical composition comprising an immune effector cell according to the sixth aspect of the invention and a pharmaceutically acceptable excipient.

The present invention also provides, in an eighth aspect, a method of generating an immune effector cell comprising a CAR according to the first aspect of the invention, comprising introducing into an immune effector cell a vector according to the fourth aspect of the invention.

The present invention also provides, in a ninth aspect, a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention, for use in the treatment of cancer, wherein the cancer comprises cells which express an aberrantly glycosylated MUC1 protein.

The present invention also provides, in a tenth aspect, use of a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention in the manufacture of a medicament for the treatment of cancer, wherein the cancer comprises cells which express an aberrantly glycosylated MUC1 protein.

The present invention also provides, in an eleventh aspect, a method for the treatment of cancer in a subject in need thereof, comprising administering to the subject a therapeutically effective amount of a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention, wherein the cancer comprises cells which express an aberrantly glycosylated MUC1 protein.

The present invention also provides, in a twelfth aspect, a method for increasing the cytotoxicity in cancer cells that express an aberrantly glycosylated MUC1 protein in a subject having cancer, comprising administering to the subject a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention, in an amount sufficient to increase the cytotoxicity in cancer cells that express an aberrantly glycosylated MUC1 compared to the cytotoxicity of the cancer cells that express an aberrantly glycosylated MUC1 protein prior to the administration.

The present invention also provides, in a thirteenth aspect, a method for decreasing the number of cancer cells expressing an aberrantly glycosylated MUC1 protein in a subject having cancer, comprising administering to the subject a therapeutically effective amount of a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention, wherein the therapeutically effective amount is sufficient to decrease the number of cancer cells that express an aberrantly glycosylated MUC1 protein compared to the number of the cancer cells that express an aberrantly glycosylated MUC1 protein prior to the administration.

The present invention also provides, in a fourteenth aspect, a CAR according to the first aspect of the invention, a polypeptide according to the second aspect of the invention, a polynucleotide according to the third aspect of the invention, a vector according to the fourth aspect of the invention, an immune effector cell according to the sixth aspect of the invention, or a pharmaceutical composition according to the seventh aspect of the invention, for use in therapy.

### BRIEF DESCRIPTION OF THE FIGURES

FIG. 1—Schematic representation of MUC1 expression on normal vs. cancerous mucosa/epithelium. Upon tumor progression, differential expression of MUC1 on cancerous cells occurs leading to strong over-expression, failure to control apical distribution (loss of polarity), and aberrant hypoglycosylation.

FIG. 2—Binding of positive control proteins to TnMUC1 peptide (36-2) vs unglycosylated MUC1 (36-4).

FIGS. 3A-3C—Binding profiles for humanized anti-TnMUC1 scFv protein supernatants.

FIG. 4—SDS-PAGE analysis of purified anti-TnMUC1 scFv constructs.

FIG. 5—Schematic representation of MUC1 peptides tested in BIAcore assays detailing the positions of Tn or STn sugar residues: MUC1 peptide (SEQ ID NO: 97), TnMUC1 peptide (fully glycosylated; SEQ ID NO: 96), TnMUC1 peptide 1 (SEQ ID NO: 98), TnMUC1 peptide 2 (SEQ ID NO: 99), STnMUC1 peptide (SEQ ID NO: 100).

FIG. 6—Data table summarizing kₐ (1/Ms), k_{d} (1/s), and K_{D} (M) for humanized scFv proteins and murine 5E5 scFv protein for binding to TnMUC1 and STnMUC1 peptides

FIG. 7—Binding profiles and affinity of humanized anti-TnMUC1 scFvs binding to TnMUC1 peptide and MUC1 peptide.

FIG. 8—Binding of murine 5E5 scFv and humanized scFv proteins to fully glycosylated and differentially glycosylated TnMUC1 peptide and MUC1 peptide: MUC1 peptide (SEQ ID NO: 97), TnMUC1 peptide (fully glycosylated; SEQ ID NO: 96), TnMUC1 peptide 1 (SEQ ID NO: 98), TnMUC1 peptide 2 (SEQ ID NO: 99).

FIG. 9—Binding profiles and affinity of humanized anti-TnMUC1 scFv proteins to both STnMUC1 and MUC1 peptides.

FIG. 10—Binding of humanized anti-TnMUC1 scFv protein 13P16 to various MUC1 peptides: MUC1 peptide (SEQ ID NO: 97), TnMUC1 peptide (fully glycosylated; SEQ ID NO: 96), TnMUC1 peptide 1 (SEQ ID NO: 98), TnMUC1 peptide 2 (SEQ ID NO: 99), STnMUC1 peptide (SEQ ID NO: 100).

FIG. 11—Fitted curves of humanized scFv binding in TnMUC1-positive MDA-MB-468 cells (representative data from n=1 experiment).

FIG. 12—Fitted curves of humanized scFv binding in TnMUC1-negative PC3 cells (representative data from n=1 experiment).

FIG. 13—Fitted curves of humanized scFv binding in highly TnMUC1-positive Jurkat cells (MFI values at top concentration 16P4 (82A) and 13P18 (19A) removed prior to curve fitting due to evidence of a hook effect (representative data from n=1 experiment)).

FIG. 14—Fitted binding curves of humanized scFv 13P16 (97A) binding in Jurkat, MDA-MB-468 cells and PC3 cell (standard error bars based on data from n=2 experiments).

FIG. 15—Percent expression of TnMUC1 on the surface of Jurkat, MDA-MB-468 and PC3 cells.

FIGS. 16A-16D—Plasma membrane protein array spotting patterns: FIG. 16A: control spotting pattern; FIGS. 16B-16D: untransduced and BCMA CAR-T transduced cells from various donors.

FIG. 17—Plasma membrane protein array spotting patterns using anti-human MUC1 mAb and controls.

FIGS. 18A-18D—Plasma membrane protein array spotting patterns (pre-screen study) using untransduced cells and TnMUC1 transduced cells. FIG. 18A: control spotting pattern; FIG. 18B: untransduced T cells; FIG. 18C: BCMA CAR-T cells; and FIG. 18D: TnMUC1 CAR-T cells.

FIGS. 19A-19D—Plasma membrane protein array patterns (confirmatory screen). FIG. 19A: control spotting pattern; FIG. 19B: untransduced cells; FIG. 19C BCMA CAR-T cells; and FIG. 19D: TnMUC1 CAR-T cells.

FIGS. 20A-20C—Basal phenotyping of untransduced and transduced 5E5 CAR-T cells. FIG. 20A shows transduction efficiency (as a percentage expression and degree of expression);

FIG. 20B shows average CD4+/CD8+ ratios; FIG. 20C shows CAR-T cell activation (CD69+41BB+) and exhaustion (PD1+LAG3+ TIM3+) status in CD4+ vs CD8+ CAR-T cells, compared to untransduced T cells.

FIG. 21—Heatmaps of n=3 donors from FIG. 20 showing the percentages of T cell memory subsets of all tested samples.

FIGS. 22A-22C—Evaluation of basal activation in unstimulated CAR-T cells: FIG. 22A: CD4/CD8 ratios; FIG. 22B: activation/exhaustion status depicting CD69+ TIM3+PD1+ Cells in T-cell subpopulations; FIG. 22C: supernatant cytokine and granzyme B release. All graphs show the average response with error bars indicating standard deviation. Scatter plots with bars show the spread of individual donor responses represented by different colored circles.

FIGS. 23A-23B—Evaluation of antigen independent signaling for TnMUC-1-BBζ(PGK) CAR-T cells compared to CD19-BBζ(PGK) (MB049) and GD2-28ζ(EF1a) (MB62): FIG. 23A: normalized pCD3ζ signaling in relation to total CD3ζ and GAPDH loading control; FIG. 23B normalized pZAP70 signaling in relation to GAPDH loading control.

FIGS. 24A-24B—Detection of CAR-T signaling transduction using capillary western (PEGGY-SUE). FIG. 24A: Western data representing activation profile of GD2-28ζ(EF1a) (MB062) CAR-T cell compared to CD19-BBζ (MB049) and TnMUC-1-BBζ (MB040) CAR-T cells, with an increase in pCD3ζ, pZAP70, pERK1/2, and a decrease in total IκBα; FIG. 24B: calculated normalized pCD3ζ signal showing significantly lower activation of CD19-BBζ (MB049), TnMUC-1-BBζ (MB040) CAR-T cells in comparison to GD2-28ζ(EF1a) (MB062) CAR-T cells.

FIG. 25—MSD assay measuring TnMUC1 CAR-T cell activation (via IFNγ release) in the presence of TnMUC1 PC3 tumor cell lines (24 hours post co-culture, data is mean of n=4 donors).

FIGS. 26A-26C—Analysis of TnMUC1 CAR-T mediated killing of PC3 TnMUC1 cells. FIG. 26A shows target density dependent rate of killing by MB024, with high to low target expression relating to quickest to slowest killing respectively; FIG. 26B shows statistical significance measurement of percent live cells at 72 hours in PC3 TnMUC1 positive cells relative to PC3 WT control; and FIG. 26C shows activation measured by IFNγ release at 24 hours by MSD.

FIG. 27—Normalized IncyCyte CAR-T killing response on PC3.wt and COSMC knockout cell lines: Normalized kinetic TnMUC-1 CAR-T cell (MB051, MB052, MB053 and MB054) killing responses showing differential rate and threshold of killing of the PC3.wt (TnMUC-1 negative), PC3.4C11(5% TnMUC-1) and PC3.5F5 (100% TnMUC1) cell lines depending on the level of target expression (n=9).

FIGS. 28A-28D—Kt50 and % live cells obtained at threshold endpoints on PC3.wt and COSMC knockout cell lines: Bonferroni one-way ANOVA test for MB051, MB052, MB053 and MB054 where, FIG. 28A shows calculated Kt50 for the CAR-T cells on TnMUC-1 expressing cell lines, FIG. 28B shows % live cells on the TnMUC-1 high expressing PC3.5F5 cell line, FIG. 28C shows % live cells on the TnMUC-1 low expressing PC3.4C11 cell line and FIG. 28D shows % live cells on the TnMUC-1 negative cell line (PC3.wt) (n=9).

FIG. 29—Peptide stimulation IFNγ activation by CAR MB024: plots showing IFNγ released by CAR MB024 upon co-culture with the fully glycosylated TnMUC1 peptide (SEQ ID NO: 96), as well as the partially glycosylated TnMUC1 peptide 1 (SEQ ID NO: 98) and STnMUC1 peptide (SEQ ID NO: 100). No IFNγ was seen when CAR T-cells were cultured in the presence of MUC1 peptide (SEQ ID NO: 97) or TnMUC1 peptide 2 (SEQ ID NO: 99). IFNγ analysis was carried out by MSD.

FIG. 30—CAR T cell cytokine and granzyme B release in response to co-culture with Jurkat WT and Jurkat COSMC+ cell lines: Cytokine release from Humanised 5E5 CAR T cells in response to 24 hrs co-culture with Jurkat WT and Jurkat COSMC+ tumour cell lines. Dot plot graphs show the average response with error bars indicating standard deviation. MB021, MB022 & MB024 T cell only conditions N=8 (4 donors in duplicate), MB025 T cells only N=6 (3 donors in duplicate), CAR T cells+COSMC−/COSMC+ conditions N=4 (single repeats in 4 donors), COSMC− & COSMC+ target cells only N=4. UT=untransduced T cells; COSMC(−)=Jurkat WT & COSMC(+)=JurkatCOSMC+ tumour cell lines.

FIGS. 31A-31C—Flow cytometry analysis of Humanised 5E5 CAR T cells co-cultured with MDA-MB-468 and MC7F MUC1 KO tumour cell lines for 24 hrs. FIG. 31A: The transduction efficiency and CD4+/CD8+ ratio of Humanised 5E5 CAR T cells cultured alone and in co-culture after 24 hrs. FIGS. 31B-31C. Intracellullar cytokine levels in Humanised 5E5 CAR T cells cultured alone and in co-cultures after 24 hrs; FIG. 31B: represents the percentage of CAR T cells positive for intracellular cytokine and FIG. 31C: the degree of intracellular cytokine positivity as measured by median fluorescence intensity (MFI). N=4 donors tested for MB021, MB022, MB024 and N=3 for MB025. All graphs show the average response with error bars indicating standard deviation. Scatter plots with bars show the spread of individual donor responses represented by different coloured circles. 468=MDA-MB-68 and KO=MCF7-KO tumour cell lines.

FIGS. 32A-32B—Supernatant cytokine analysis of Humanised 5E5 CAR T cells co-cultured with MDA-MB-468 and MC7F MUC1 KO tumour cell lines for 24 hrs. FIG. 32A: Supernatant Cytokine Bead Array (CBA) assay results. FIG. 32B: Luminex assay results. N=4 donors tested for MB021, MB022, MB024 and N=3 for MB025. All graphs show the average response with error bars indicating standard deviation. Scatter plots with bars show the spread of individual donor responses represented by different coloured circles. 468=MDA-MB-68 and KO=MCF7-KO tumour cell lines.

FIG. 33—Cell surface general MUC1 and specific Tn/STnMUC1 expression levels of MDA-MB-468 & MCF7-KO tumour cell lines were evaluated prior to the intracellular staining to confirm their expression specificity. Gating was based on BV421 secondary antibody only control. Arrows showed the respective positive populations of MUC1 and Tn/STnMUC1.

FIG. 34—Proliferation of TnMUC1 CAR T cells in response to Tn-MUC1 expressing cancer cell lines: proliferation response based on the division index (left panel); and % of CAR T and T cell proliferation (right panel). 4 donors were tested in TnMUC CAR T & UT T cell conditions and 3 donors for BCMA CAR T. All plots show the average with standard deviation. P values=ns (no significance)>0.05 *<0.05 **<0.01 ***<0.001. TA=TransACT. Fab(2)=Tn-MUC1 CAR.

FIGS. 35A-35C—Evaluating Tn-MUC1 CAR expression and CD4/CD8 ratios in response to co-culture with Tn-MUC1 expressing cancer cell lines. FIG. 35A: % frequency of CAR positive T cells. FIG. 35B: Level of CAR expression on the T cell surface based on medium fluorescence intensity. FIG. 35C: Graph showing the CD4/CD8 ratio in CAR positive cells (Tn-MUC1 & BCMA CAR T) compared to UT T cells (CAR negative). 4 donors were tested in TnMUC1 CAR & UT T cell conditions and 3 donors for BCMA CAR T. All plots show the average with standard deviation P values=ns (no significance)>0.05 *<0.05 **<0.01 ***<0.001. TA=TransACT. Fab(2)=TnMUC1 CAR detection.

FIG. 36—Quantification of IFN-γ production. Supernatants removed from co-culture plates were analysed by MSD to quantify the production of IFN-γ by CAR T cell populations. Four donors were tested. Error bars display Mean with SD (N=3). Data is displayed on a Log 10 Scale.

FIG. 37—Tumour volume in NSG mice inoculated with PC3 5F5 human prostate cell line and treated with TnMUC1 CAR T cells or control BCMA CAR T cells or PBS (vehicle). 11 days after initial tumour measurement, tumours were dosed with PBS (no T cells), BCMA (control CAR) or TnMUC1 CAR T cells at a dose of 1×10⁷. Graph shows tumour volume (in mm³) on study day 31 (25 days after initial tumour measurement; 14 days post T cell dosing). One-way ANOVA followed by Tukey post-hoc test was performed. Error bars indicate standard deviation. ns>0.05, *** p<0.001.

FIGS. 38A-38B—IFN-γ, IL-2 and TNF-α release measured in blood serum of PC3 5F5 tumour-bearing CDX mice prior to (pre-treatment—D0) and 7 days post-dosing (post-treatment—D7) with PBS (no T cells), BCMA CAR T cells (control CAR) or TnMUC1 CAR T cells. FIG. 38A: Secreted levels of IFN-γ, IL-2 and TNF-α are shown in pg/ml (y-axis). Each dot is cytokine concentration at a given time-point for a given mouse. Points for the same mouse are connected by dotted lines. Data is presented in a log 10 transformation on the y-axis. Wherever data is <LLoQ for one time-point, this has been set to zero for illustration purpose. FIG. 38B: Direct comparison of each cytokine (IFN-γ, IL-2 and TNF-α) using Bayesian linear regression indicating that TnMUC1 CAR T group leads to higher levels of all three cytokines from pre-treatment (DO) to post-treatment (D7) (for each of “Pre-treatment” and “post-treatment” data plotted from left to right is shown for BCMA CAR T, PBS, and TnMUC1 CAR T).

FIGS. 39A-39B—IFNγ secretion by TnMUC1 CAR T and UT T cells at increasing levels of Tn-MUC1 positive cells in Jurkat and PC-3 co-culture supernatants. FIG. 39A: Mean IFNγ secretion of TnMUC1 CAR T and UTT cells plotted at each cell line condition. Data is represented as log 10 transformed IFNγ concentration (pg/mL). Effector only represents TnMUC1 CAR T cells cultured in the absence of target cells. FIG. 39B: Comparison of TnMUC1 CAR T cells to UT T cells at each cell line condition, calculated as a ratio. Ratios greater than 1 (dotted line) indicate higher IFNγ release in TnMUC1 CAR T cells than UT T cells. Effector only represents TnMUC1 CAR T cells cultured in the absence of target cells. Data is a mean of three donors, error bars present 95% confidence intervals.

### DETAILED DESCRIPTION OF THE INVENTION

### Overview

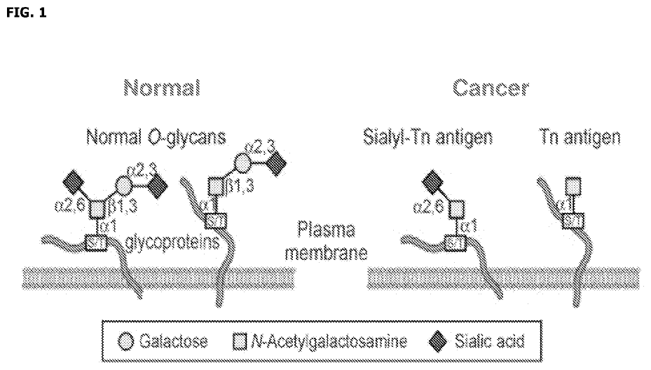

MUC1 is a transmembrane glycoprotein expressed on apical surface of normal cells and characterised by extensive O-glycosylation (FIG. 1, left). Together with other mucin family members MUC1 forms a mucus, protective layer against pathogenic and environmental challenges (Hattrup and Gendler, Annu Rev Physiol. 2008; 70: 431-57). In cancers, MUC1 is often overexpressed by tumour cells, loses its polarity and, importantly, MUC1 becomes aberrantly glycosylated with truncated glycans (FIG. 1, right). The most prevalent truncated glycans found in cancer are the Thomsen-nouveau (Tn) and sialyl-Tn (STn) glycoforms (Springer, 1984). The terms “aberrantly glycosylated MUC1 protein” and “AG-MUC1” as used herein may refer collectively to all aberrantly glycosylated MUC1 proteins or to individual variants of aberrantly glycosylated MUC1 proteins, e.g., TnMUC1, STnMUC1, etc.

Increased levels of AG-MUC1 have been associated with cancer progression and metastasis that correlate with poor prognosis and high mortality (Cascio and Finn, 2016; Finn et al., 2011). Striking difference in expression levels and glycosylation status between healthy and tumour tissues makes aberrantly glycosylated Tn/STn-MUC1 an attractive target for CAR T cell therapy.

The present invention generally relates to improved chimeric antigen receptors (CAR) targeted to these AG-MUC1 proteins. The improved CAR molecules of the invention are intended to be used in compositions and methods for preventing or treating cancers that express AG-MUC1 proteins, thereby preventing, treating, or ameliorating at least one symptom associated with an AG-MUC1 protein expressing cancer. In particular embodiments, the invention relates to improved cell therapy of cancers that express AG-MUC1 protein, using genetically modified immune effector cells.

The improved compositions and methods of adoptive cell therapy contemplated herein, provide genetically modified immune effector cells that can readily be expanded, exhibit long term persistence in vivo, and demonstrate antigen dependent cytotoxicity to cells expressing AG-MUC1.

Particularly, a specific fast off-rate and lower affinity, but high potency of the CAR-T cells can minimize the likelihood of off-target, off-tumour toxicities previously observed in other gene-engineered T-cell therapies and may prevent T cell exhaustion following infusion which has been described as a risk for CARs with slow off-rates.

One of the key requirements for CAR molecules is the ability to differentiate tumour tissues from healthy tissues. Previously, it has been shown that CARs with high affinity can lead to collateral targeting of healthy tissues resulting in on/off-target, off-tumour toxicity (Johnson et al., 2015; Park et al., 2017; Watanabe et al., 2018). Therefore, we compared slow off-rate and fast off-rate CAR-Ts on their ability to discriminate between TnMUC1-positive and -negative cells. Both fast and slow off-rate CAR-Ts secreted IFNγ in response to TnMUC1-positive cell lines, however slow off-rate CAR-Ts produced IFNγ in response to tumour cells lacking the MUC1 protein, whereas fast off-rate CAR-Ts did not. These data suggest that fast off-rate CAR-T cells specifically recognise TnMUC1 target, minimizing the risk of off-target:off-tumour toxicities in patients.

### Chimeric Antigen Receptor (CAR)

In various embodiments, genetically engineered receptors that redirect immune effector cells toward cancer cells expressing an AG-MUC1 protein are provided. These genetically engineered receptors, referred to herein as chimeric antigen receptors (CARs), are molecules that combine antibody-based specificity for a desired antigen (e.g., AG-MUC1) with a T cell receptor-activating intracellular domain to generate a chimeric protein that exhibits a specific anti-AG-MUC1 cellular immune activity. The term “chimeric” describes being composed of parts of different proteins or DNAs from different origins.

In particular embodiments, CARs comprise an extracellular domain (also referred to as a binding domain, antigen binding domain, or antigen-specific binding domain) that binds to AG-MUC1 proteins, a transmembrane domain, one or more costimulatory domains and one or more intracellular signalling domains. Engagement of the anti-AG-MUC1 antigen binding domain of the CAR with the antigen on the surface of a target cell results in clustering of the CAR and delivers an activation stimulus to the CAR containing cell. The main characteristic of CARs is their ability to redirect immune effector cell specificity, thereby triggering proliferation, cytokine production, phagocytosis and/or production of molecules that can mediate cell death of the target antigen expressing cell in a major histocompatibility (MHC) independent manner, exploiting the cell specific targeting abilities of monoclonal antibodies, soluble ligands or cell specific co-receptors.

### Extracellular Domain

In various embodiments, a CAR comprises an extracellular binding domain that comprises an anti-AG-MUC1 antigen binding domain; a transmembrane domain; one or more co-stimulatory signalling domains; and one or more intracellular signalling domains.

In particular embodiments, the CAR further comprises a hinge domain between the antigen binding domain and the intracellular signalling domain. The CAR may also comprise hinge domains or spacer domains between any of the extracellular binding domain, the transmembrane domain, the costimulatory domains and/or the intracellular signalling domains.

In particular embodiments, CARs comprise an extracellular binding domain that comprises an anti-AG-MUC1 antigen binding domain that specifically binds to an AG-MUC1 protein expressed on a target cell, e.g., a cancer cell. As used herein, the terms, “binding domain,” “extracellular domain,” “extracellular binding domain,” “antigen binding domain,” “antigen-specific binding domain,” and “extracellular antigen specific binding domain,” are used interchangeably and provide a CAR with the ability to specifically bind to the target antigen of interest, e.g., AG-MUC1. The binding domain may be derived either from a natural, synthetic, semi-synthetic, or recombinant source.

In particular embodiments, an anti-AG-MUC1 antigen binding domain is an antibody or antigen binding domain thereof.

The terms “specific binding affinity” or “specifically binds” or “specifically bound” or “specific binding” or “specifically targets” as used herein, describe binding of an anti-AG-MUC1 antigen binding domain (or a CAR comprising the same) to AG-MUC1 at a greater binding affinity than background binding. A binding domain (or a CAR comprising a binding domain or a fusion protein containing a binding domain) “specifically binds” to an AG-MUC1 protein if it binds to or associates with AG-MUC1 with an affinity or Ka (i.e., an equilibrium association constant of a particular binding interaction with units of I/M) of, for example, greater than or equal to about 10⁵ M⁻¹. In certain embodiments, a binding domain (or a fusion protein thereof) binds to a target with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, or 10¹² M⁻¹. “High affinity” binding domains (or single chain fusion proteins thereof) refers to those binding domains with a Ka of at least 10⁷ M⁻¹, or at least 10⁸ M⁻¹, or at least 10⁹ M⁻¹, or at least 10¹⁰ M⁻¹, or at least 10¹¹ M⁻¹, or at least 10¹² M⁻¹ or greater.

Alternatively, affinity may be defined as an equilibrium dissociation constant (K_{D}) of a particular binding interaction with units of M (e.g., 10⁻⁵ M to 10⁻¹³ M, or less). Affinities of binding domain polypeptides and CAR proteins according to the present disclosure can be readily determined using conventional techniques, e.g., by competitive ELISA (enzyme linked immunosorbent assay), or by binding association, or displacement assays using labelled ligands, or using a surface-plasmon resonance device such as the Biacore T 100, which is available from Biacore, Inc., Piscataway, NJ, or optical biosensor technology such as the EPIC system or EnSpire that are available from Corning and Perkin Elmer respectively (see also, e.g., Scatchard, Ann NY Acad Sci. 1949; 51(4): 660; and U.S. Pat. No. 5,283,173; or the equivalent).

K_{D}/K_{A}/k_{d}/kₐ

In an embodiment, the equilibrium dissociation constant (K_{D}) of the CAR:AG-MUC1 interaction is 500 nM or less, 200 nM or less, 100 nM or less, 10 nM or less, 2 nM or less or 1 nM or less. Alternatively, the K_{D} may be between 5 nM and 10 nM; or between 1 nM and 2 nM. The K_{D} may be between 1 pM and 500 pM; or between 500 pM and 1 nM. A skilled person will appreciate that the smaller the K_{D} numerical value, the stronger the binding. The reciprocal of K_{D} (i.e. 1/K_{D}) is the equilibrium association constant (K_{A}) having units M⁻¹. A skilled person will appreciate that the larger the K_{A} numerical value, the stronger the binding.

The dissociation rate constant (k_{d}) or “off-rate” describes the stability of the CAR:AG-MUC1 complex, i.e., the fraction of complexes that decay per second. For example, a k_{d} of 0.01 s⁻¹ equates to 1% of the complexes decaying per second. In an embodiment, the dissociation rate constant (k_{d}) is 1×10⁻² s⁻¹ or less, 1×10⁻³ s⁻¹ or less, 1×10⁻⁴ s⁻¹ or less, 1×10⁻⁵ s⁻¹ or less, or 1×10⁻⁶ s⁻¹ or less. The k_{d} may be between 1×10⁻⁵ s⁻¹ and 1×10⁻⁴ s⁻¹; between 1×10⁻⁴ s⁻¹ and 1×10⁻³ s⁻¹; or between 1×10⁻³ s⁻¹ and 1×10⁻² s⁻¹.

The association rate constant (kₐ) or “on-rate” describes the rate of CAR:AG-MUC1 complex formation. In an embodiment, the association rate constant (kₐ) is 1×10⁶ M⁻¹ s⁻¹ or less, 1×10⁵ M⁻¹s⁻¹ or less, 1×10⁴ M⁻¹s⁻¹ or less, 1×10³ M⁻¹s⁻¹ or less, or 1×10² M⁻¹s⁻¹ or less. The kₐ may be between 1×10⁶ M⁻¹s⁻¹ and 1×10⁵ M⁻¹s⁻¹; between 1×10⁴ M⁻¹s⁻¹ and 1×10³ M⁻¹s⁻¹; and between 1×10³ M⁻¹s⁻¹ and 1×10² M⁻¹s⁻¹.

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In particular embodiments, the extracellular binding domain of a CAR comprises an antibody or antigen binding domain thereof. An “antibody” refers to a binding agent that is a polypeptide comprising at least a light chain or heavy chain immunoglobulin variable region which specifically recognizes and binds an epitope of an antigen, such as a lipid, carbohydrate, polysaccharide, glycoprotein, peptide, or nucleic acid containing an antigenic determinant, such as those recognized by an immune cell.

An “antigen (Ag)” refers to a compound, composition, or substance that can stimulate the production of antibodies or a T cell response in an animal, including compositions (such as one that includes a cancer-specific protein) that are injected or absorbed into an animal. Exemplary antigens include but are not limited to lipids, carbohydrates, polysaccharides, glycoproteins, peptides, or nucleic acids. An antigen reacts with the products of specific humoral or cellular immunity, including those induced by heterologous antigens, such as the disclosed antigens. In particular embodiments, the target antigen is an AG-MUC1 antigen.

An “epitope” or “antigenic determinant” refers to the region of an antigen to which a binding agent binds.

Antibodies include antigen binding domains thereof, such as Camel Ig, Ig NAR, Fab fragments, Fab′ fragments, F(ab)′₂ fragments, F(ab)′₃ fragments, Fv, single chain Fv proteins (“scFv”), bis-scFv, (scFv)₂, minibodies, diabodies, triabodies, tetrabodies, disulphide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding domains thereof. See also, Pierce Catalog and Handbook, 1994-1995 (Pierce Chemical Co., Rockford, IL); Kuby, J., Immunology, 3^{rd} Ed., W. H. Freeman & Co., New York, 1997.

As would be understood by the skilled person and as described herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ and μ. Mammalian light chains are classified as δ or κ.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

Immunoglobulins comprising the α, δ, ε, γ and μ heavy chains are classified as immunoglobulin IgA, IgD, IgE, IgG, and IgM. The complete antibody forms a “Y” shape. The stem of the Y consists of the second and third constant regions (and for IgE and IgM, the fourth constant region) of two heavy chains bound together and disulphide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” “CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature. 1989; 342(6252): 877-83. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Accordingly, an antigen binding protein is provided, which comprises any one or a combination of the following CDRs:

CDRH1 of SEQ ID NO: 31
(DHAIH), (or CDRH1 of SEQ ID NO: 13, 19, 25, 37, or
43, each of which is identical to SEQ ID NO: 13);
CDRH2 of SEQ ID NO: 32
(HFSPGNTDIKYNDKFKG), (or CDRH1 of SEQ ID NO: 14,
20, 26, 38, or 44, each of which is identical to
SEQ ID NO: 32);
CDRH3 of SEQ ID NO: 33
(STFFFDY), (or CDRH3 of SEQ ID NO: 15, 21, 27, 39,
or 45, each of which is identical to SEQ ID NO: 33);
CDRL1 of SEQ ID NO: 28
(KSSQSLLNSGDQKNYLT), (or CDRL1 of SEQ ID NO: 10,
16, 22, 34, or 40, each of which is identical to
SEQ ID NO: 28);
CDRL2 of SEQ ID NO: 29
(WASTRES), (or CDRL2 of SEQ ID NO: 11, 17, 23, 35,
or 41, each of which is identical to SEQ ID NO: 29);
CDRL3 of SEQ ID NO: 30
(QNDYSYPLT), (or CDRL3 of SEQ ID NO: 12, 18, 24, 36,
or 42, each of which is identical to SEQ ID NO: 30);
OR
CDRH1, CDRH2, CDRH3 from SEQ ID NO: 53 (or CDRH1,
CDRH2, CDRH3 from SEQ ID NO: 47, 49, 51, 55, or 57);
CDRL1, CDRL2, CDRL3 from SEQ ID NO: 52 (or CDRL1,
CDRL2, CDRL3 from SEQ ID NO: 48, 50, 54, or 56).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen.

References to “VL” or “VL” refer to the variable region of an immunoglobulin light chain, including that of an antibody, Fv, scFv, dsFv, Fab, or other antibody fragment as disclosed herein. Illustrative examples of light chain variable regions that are suitable for constructing anti-AG-MUC1 CARs contemplated herein include, but are not limited to the light chain variable region sequences set forth in SEQ ID NOS: 46, 48, 50, 52, 54, and 56.

References to “VH” or “VH” refer to the variable region of an immunoglobulin heavy chain, including that of an antibody, Fv, scFv, dsFv, Fab, or other antibody fragment as disclosed herein. Illustrative examples of heavy chain variable regions that are suitable for constructing anti-AG-MUC1 CARs contemplated herein include, but are not limited to the heavy chain variable region sequences set forth in SEQ ID NOS: 47, 49, 51, 53, 55, and 57.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular preferred embodiments, a CAR comprises antigen-specific binding domain that is a chimeric antibody or antigen binding domain thereof.

In preferred embodiments, the antibody is a human antibody (such as a human monoclonal antibody) or antigen binding domain thereof that specifically binds to a human AG-MUC1 protein.

In one embodiment, a CAR comprises a “humanized” antibody or antigen binding domain thereof. A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen, et al., Proc. Natl Acad Sci USA. 1989; 86(24): 10029-10032; Hodgson, et al., Biotechnology, 1991; 9(5): 421-5). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. Nonlimiting examples of ways to produce such humanized antibodies are detailed in EP-A-0239400 and EP-A-054951.

In particular embodiments, an anti-AG-MUC1 antibody or antigen binding domain thereof, includes but is not limited to a Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

In particular embodiments, an anti-AG-MUC1 antibody or antigen binding domain thereof is a scFv.

An exemplary AG-MUC1-specific binding domain is an immunoglobulin variable region specific for AG-MUC1 that comprises at least one human framework region. A “human framework region” refers to a wild type (i.e., naturally occurring) framework region of a human immunoglobulin variable region, an altered framework region of a human immunoglobulin variable region with less than about 50% (e.g., preferably less than about 45%, 40%, 35%, 30%, 25%, 20%, 15%, 10%, 5% or 1%) of the amino acids in the region are deleted or substituted (e.g., with one or more amino acid residues of a nonhuman immunoglobulin framework region at corresponding positions), or an altered framework region of a nonhuman immunoglobulin variable region with less than about 50% (e.g., preferably less than about 45%, 40%, 35%, 30%, 25%, 20%, 15%, 10%, 5% or 1%) of the amino acids in the region deleted or substituted (e.g., at positions of exposed residues and/or with one or more amino acid residues of a human immunoglobulin framework region at corresponding positions) so that, in one embodiment, immunogenicity is reduced.

In certain embodiments, a human framework region is a wild type framework region of a human immunoglobulin variable region. In certain other embodiments, a human framework region is an altered framework region of a human immunoglobulin variable region with amino acid deletions or substitutions at one, two, three, four, five, six, seven, eight, nine, ten or more positions. In other embodiments, a human framework region is an altered framework region of a non-human immunoglobulin variable region with amino acid deletions or substitutions at one, two, three, four, five, six, seven, eight, nine, ten or more positions.

In particular embodiments, an AG-MUC1-specific binding domain comprises at least one, two, three, four, five, six, seven or eight human framework regions (FR) selected from human light chain FR1, human heavy chain FR1, human light chain FR2, human heavy chain FR2, human light chain FR3, human heavy chain FR3, human light chain FR4, and human heavy chain FR4.

Human FRS that may be present in an AG-MUC1-specific binding domains also include variants of the exemplary FRS provided herein in which one, two, three, four, five, six, seven, eight, nine, ten or more amino acids of the exemplary FRS have been substituted or deleted.

In certain embodiments, an AG-MUC1-specific binding domain comprises: (a) a humanized light chain variable region that comprises a human light chain FR1, a human light chain FR2, a human light chain FR3, and a human light chain FR4; and (b) a humanized heavy chain variable region that comprises a human heavy chain FRI, a human heavy chain FR2, a human heavy chain FR3, and a human heavy chain FR4.

AG-MUC1-specific binding domains provided herein also comprise one, two, three, four, five, or six CDRs. Such CDRs may be nonhuman CDRs or altered nonhuman CDRs selected from CDRL1, CDRL2 and CDRL3 of the light chain and CDRH1, CDRH2 and CDRH3 of the heavy chain. In certain embodiments, an AG-MUC1-specific binding domain comprises (a) a light chain variable region that comprises a light chain CDRL1, a light chain CDRL2, and a light chain CDRL3, and (b) a heavy chain variable region that comprises a heavy chain CDRH1, a heavy chain CDRH1, and a heavy chain CDRH3.

In one embodiment, an AG-MUC1-specific binding domain comprises light chain CDR sequences set forth in SEQ ID NOs: 10-12, 16-18, 22-24, 28-30, 34-36, and 40-42. In a particular embodiment, an AG-MUC1-specific binding domain comprises light chain CDR sequences with at least 85%, 86%, 87%, 88%, 89%, 90%, 91%, 92%, 93%, 94%, 95%, 96%, 97%, 98% or 99% amino acid identity to the light chain CDR sequences set forth in SEQ ID NOS: 10-12, 16-18, 22-24, 28-30, 34-36, and 40-42.

In one embodiment, an AG-MUC1-specific binding domain comprises heavy chain CDR sequences set forth in SEQ ID NOs: 13-15, 19-21, 25-27, 31-33, 37-39, and 43-45. In a particular embodiment, an AG-MUC1-specific binding domain comprises light chain CDR sequences with at least 85%, 86%, 87%, 88%, 89%, 90%, 91%, 92%, 93%, 94%, 95%, 96%, 97%, 98% or 99% amino acid identity to the heavy chain CDR sequences set forth in SEQ ID NOS: 13-15, 19-21, 25-27, 31-33, 37-39, and 43-45.

In particular embodiments, an anti-AG-MUC1 antibody or antigen binding domain thereof comprises:
a CDRL1, a CDRL2, and a CDRL3 from SEQ ID NO: 52; and
a CDRH1, a CDRH2, and a CDRH3 from SEQ ID NO: 53.

In particular embodiments, an anti-AG-MUC1 antibody or antigen binding domain thereof comprises:
a CDRL1 sequence at least 90% identical to SEQ ID NO: 28;
a CDRL2 sequence at least 90% identical to SEQ ID NO: 29;
a CDRL3 sequence at least 90% identical to SEQ ID NO: 30;
a CDRH1 sequence at least 90% identical to SEQ ID NO: 31;
a CDRH2 sequence at least 90% identical to SEQ ID NO: 32; and a CDRH3 sequence at least 90% identical to SEQ ID NO: 33.

In particular embodiments, an anti-AG-MUC1 antibody or antigen binding domain thereof comprises:
a CDRL1 sequence of SEQ ID NO: 28;
a CDRL2 sequence of SEQ ID NO: 29;
a CDRL3 sequence of SEQ ID NO: 30;
a CDRH1 sequence of SEQ ID NO: 31;
a CDRH2 sequence of SEQ ID NO: 32; and a CDRH3 sequence of SEQ ID NO: 33.

In particular embodiments, an anti-AG-MUC1 CAR antigen binding domain comprises:
(a) a VL sequence set forth in SEQ ID NO: 46 and a VH sequence set forth in SEQ ID NO: 47;
(b) a VL sequence set forth in SEQ ID NO: 48 and a VH sequence set forth in SEQ ID NO: 49;
(c) a VL sequence set forth in SEQ ID NO: 50 and a VH sequence set forth in SEQ ID NO: 51;
(d) a VL sequence set forth in SEQ ID NO: 52 and a VH sequence set forth in SEQ ID NO: 53;
(e) a VL sequence set forth in SEQ ID NO: 54 and a VH sequence set forth in SEQ ID NO: 55; or
(f) a VL sequence set forth in SEQ ID NO: 56 and a VH sequence set forth in SEQ ID NO: 57.

In some embodiments, an anti-AG-MUC1 CAR antigen binding domain is an scFv comprising, from N-terminus to C-terminus, a VH sequence and a VL sequence, wherein the VH and VL sequences are optionally separated by a linker sequence. In other embodiments, an anti-AG-MUC1 CAR antigen binding domain is an scFv comprising, from N-terminus to C-terminus, a VL sequence and a VH sequence, wherein the VH and VL sequences are optionally separated by a linker sequence.

In particular embodiments, an anti-AG-MUC1 CAR antigen binding domain comprises a scFv having a sequence at least 90% identical to a sequence set forth in SEQ ID NO: 2, SEQ ID NO: 3, SEQ ID NO: 4, SEQ ID NO: 5, SEQ ID NO: 6, or SEQ ID NO: 7.

In particular embodiments, an anti-AG-MUC1 CAR antigen binding domain comprises a scFv having a sequence set forth in SEQ ID NO: 2, SEQ ID NO: 3, SEQ ID NO: 4, SEQ ID NO: 5, SEQ ID NO: 6, or SEQ ID NO: 7.

In particular embodiments, an anti-AG-MUC1 CAR antigen binding domain comprises a scFv having a sequence at least 90% identical to a sequence selected from the group consisting of SEQ ID NOs: 90, 91, 92, 93, 94, and 95.

In particular embodiments, an anti-AG-MUC1 CAR antigen binding domain comprises a scFv having a sequence selected from the group consisting of SEQ ID NOs: 90, 91, 92, 93, 94, and 95.

In certain embodiments, an anti-AG-MUC1 CAR antigen binding domain binds one or more epitopes on an aberrantly glycosylated MUC1 protein with a K_{D} of less than 200 nanomolar (nM).

In an embodiment, the equilibrium dissociation constant (K_{D}) of the CAR:AG-MUC1 interaction is 200 nM or less. Alternatively, the K_{D} of the CAR:AG-MUC1 interaction is 1 nM to 200 nM, 10 nM to 200 nM, 20 nM to 200 nM, 50 nM to 200 nM, 100 nM to 200 nM, 150 nM to 200 nM, or 170 nM to 200 nM.

In other embodiments, an anti-AG-MUC1 CAR antigen binding domain is a humanized antibody or antigen binding domain thereof and binds one or more epitopes on an aberrantly glycosylated MUC1 protein with a dissociation rate constant (k_{off}) that is greater than the k_{off} of a non-humanised antibody or antigen binding domain thereof.

In particular embodiments, the dissociation rate constant of the anti-AG-MUC1 CAR interaction is at least 1×10⁻³ s⁻¹. For example, the dissociation rate constant (k_{off}) can be at least 1×10⁻³ s⁻¹, at least 2×10⁻³ s⁻¹, at least 3×10⁻³ s⁻¹, at least 4×10⁻³ s⁻¹, at least 5×10⁻³ s⁻¹, at least 6×10⁻³ s⁻¹, at least 7×10⁻³ s⁻¹, at least 8×10⁻³ s⁻¹, or at least 9×10⁻³ s⁻¹. In some embodiments, the dissociation rate constant (k_{off}) is 1×10⁻³ s⁻¹ to 1×10⁻² s⁻¹. In some embodiments, the dissociation rate constant (k_{off}) is 1×10⁻³ s⁻¹ to 1×10⁻² s⁻¹, 2×10⁻³ s⁻¹ to 1×10⁻² s⁻¹, 3×10⁻³ s⁻¹ to 1×10⁻² s⁻¹, 4×10⁻³ s⁻¹ to 1×10⁻² s⁻¹, 5×10⁻³ s⁻¹ to 1×10⁻² s⁻¹, or 6×10⁻³ s⁻¹ to 1×10⁻² s⁻¹. In some embodiments, the dissociation rate constant (k_{off}) is 2×10⁻³ s⁻¹ to 1×10⁻² s⁻¹. In some embodiments, the dissociation rate constant (k_{off}) is 6×10⁻³ s⁻¹ to 1×10⁻² s⁻¹.

The dissociation rate constant (k_{off}) of the CAR:AG-MUC1 interaction can be measured by Biacore. In certain embodiments, the dissociation rate constant (k_{off}) is determined by Biacore measurement of the interaction between a humanised scFv protein and a TnMUC1 peptide, such as a TnMUC1 peptide of SEQ ID NO: 96. The dissociation rate constant (k_{off}) can be measured by Biacore as described in detail in Example 4. For example, the dissociation rate constant (k_{off}) can be measured by Biacore with a TnMUC1 peptide captured on a chip surface and a humanized scFv protein flowed over the chip surface using a 300 second association followed by a 900 seconds dissociation in buffer comprising 10 mM Hepes, pH 7.6, 150 mM NaCl, 3 mM EDTA, and 0.05% polysorbate 20. The TnMUC1 peptide can be modified on the N-terminus and/or C-terminus with an appropriate functional group to allow for capture of the peptide onto the surface of a chip according to methods known in the art.

### Linkers

In certain embodiments, the CARs comprise linker residues between the various domains, e.g., between VH and VL domains, added for appropriate spacing and conformation of the molecule. In particular embodiments the linker is a variable region linking sequence. A “variable region linking sequence,” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, transmembrane domains, co-stimulatory domains, and/or intracellular signalling domains. CARs comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)n, where n is an integer of at least one, two, three, four, or five; glycine-alanine polymers; alanine-serine polymers; and other flexible linkers known in the art.

### Spacer Domain

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 domains of IgG1, IgG4, or IgD.

### Hinge Domain

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

Illustrative hinge domains suitable for use in the CARs described herein include the hinge region derived from the extracellular regions of type I membrane proteins such as CD8α, and CD4, which may be wild-type hinge regions from these molecules or may be altered. In another embodiment, the hinge domain comprises a CD8α hinge region.

### Transmembrane Domain

The “transmembrane domain” (TM) is the portion of the CAR that fuses the extracellular binding portion and costimulatory domain/intracellular signalling domain and anchors the CAR to the plasma membrane of the immune effector cell. The TM domain may be isolated or derived either from a natural, synthetic, semi-synthetic, or recombinant source. The TM domain may be isolated or derived from (i.e., comprise) at least the transmembrane region(s) of alpha or beta chain of the T-cell receptor, CD3δ, CD3ε, CD3γ, CD3ζ, CD4, CD5, CD8α CD9, CD16, CD22, CD27, CD28, CD33, CD37, CD45, CD64, CD80, CD86, CD134, CD137 (4-1BB), CD152, CD154, CD278 (ICOS), or PD1. In a particular embodiment, the TM domain is synthetic and predominantly comprises hydrophobic residues such as leucine and valine.

In one embodiment, the CARs comprise a TM domain isolated or derived from CD8α. In another embodiment, a CAR comprises a TM domain derived from CD8α and a short oligo- or polypeptide linker, preferably between 1, 2, 3, 4, 5, 6, 7, 8, 9, or 10 amino acids in length that links the TM domain and the costimulatory/intracellular signalling domain of the CAR. A glycine-serine based linker provides a particularly suitable linker.

### Intracellular Signalling Domains

In particular embodiments, CARs comprise an intracellular signalling domain. An ‘intracellular signalling domain,” refers to the part of a CAR that participates in transducing the message of effective anti-Ag-MUC1 CAR binding to an AG-MUC1 protein into the interior of the immune effector cell to elicit effector cell function, e.g., activation, cytokine production, proliferation and/or cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity including the secretion of a cytokine. Thus, the term “intracellular signalling domain” refers to the portion of a protein which transduces the effector function signal and that directs the cell to perform a specialized function. While usually the entire intracellular signalling domain can be employed, in many cases it is not necessary to use the entire domain. To the extent that a truncated portion of an intracellular signalling domain is used, such truncated portion may be used in place of the entire domain as long as it transduces the effector function signal.

The term intracellular signalling domain is meant to include any truncated portion of the intracellular signalling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of signalling domains: intracellular signalling domains that initiate antigen-dependent primary activation through the TCR (e.g., a TCR/CD3 complex) and co-stimulatory signalling domains that act in an antigen-independent manner to provide a secondary or co-stimulatory signal. In preferred embodiments, a CAR comprises one or more “co-stimulatory signalling domains” and one or more “intracellular signalling domains”

Intracellular signalling domains regulate primary activation of the TCR complex either in a stimulatory way, or in an inhibitory way. Intracellular signalling domains that act in a stimulatory manner may contain signalling motifs which are known as immunoreceptor tyrosine-based activation motifs or ITAMs.

Illustrative examples of ITAMs containing intracellular signalling domains that are suitable for use in particular embodiments include those isolated or derived from FcRγ, FcRβ, CD3γ, CD3ε, CD3δ, CD3ζ, CD22, CD66d, CD79a, or CD79b. In particularly preferred embodiments, a CAR comprises a CD3ζ intracellular signalling domain and one or more co-stimulatory signalling domains. The intracellular signalling and co-stimulatory signalling domains may be linked in any order in tandem to the carboxyl terminus of the transmembrane domain optionally separated from the carboxyl terminus of the transmembrane domain by a linker region.

### Costimulatory Domains

In particular embodiments, CARs comprise one or more co-stimulatory signalling domains to enhance the efficacy and expansion of T cells expressing CAR receptors. As used herein, the term, “co-stimulatory signalling domain,” or “co-stimulatory domain”, refers to an intracellular signalling domain of a co-stimulatory molecule. Co-stimulatory molecules are cell surface molecules other than antigen receptors or FC receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TLR1, TLR2, TLR3, TLR4, TLR5, TLR6, TLR7, TLR8, TLR9, TLR10, TRIM, and ZAP70.

In one embodiment, a CAR comprises one or more co-stimulatory signalling domains isolated or derived from a costimulatory molecule selected from the group consisting of CD28, CD134 (OX40), and CD137 (4-1-BB).

In another embodiment, a CAR comprises a co-stimulatory signalling domain isolated or derived from CD137 (4-1BB) and an intracellular signalling domain isolated or derived from CD3ζ.

In particular embodiments, anti-AG-MUC1 CARs contemplated herein include but are not limited to the amino acid sequences set forth in SEQ ID NOS: 58, 59, 60, and 61.

In one embodiment, an anti-AG-MUC1 CAR comprises the sequence of SEQ ID NO: 58.

In one embodiment, an anti-AG-MUC1 CAR comprises the sequence of SEQ ID NO: 59.

In one embodiment, an anti-AG-MUC1 CAR comprises the sequence of SEQ ID NO: 60.

In one embodiment, an anti-AG-MUC1 CAR comprises the sequence of SEQ ID NO: 61.

In some embodiments, a CAR comprises:
a) an extracellular domain which comprises an antibody or antigen binding domain thereof that binds one or more epitopes on an aberrantly glycosylated MUC1 protein, wherein the antibody or antigen binding domain thereof binds said epitope with a K_{D} of less than 200 nanomolar (nM);
b) a transmembrane domain;
c) one or more costimulatory domains; and
d) one or more intracellular signalling domains.

In some embodiments, a CAR comprises:
a) an extracellular domain which comprises an antibody or antigen binding domain thereof that binds one or more epitopes on an aberrantly glycosylated MUC1 protein, wherein the antibody or antigen binding domain thereof binds said epitope with a dissociation rate constant (k_{off}) of at least 1×10⁻³ s⁻¹;
b) a transmembrane domain;
c) one or more costimulatory domains; and
d) one or more intracellular signalling domains.

In some embodiments, a CAR comprises:
a) an extracellular domain which comprises a humanised antibody or antigen binding domain thereof that binds one or more epitopes on an aberrantly glycosylated MUC1 protein, wherein the antibody or antigen binding domain thereof comprises:
   a CDRL1 sequence at least 90% identical to SEQ ID NO: 28, preferably a CDRL1 sequence of SEQ ID NO: 28;
   a CDRL2 sequence at least 90% identical to SEQ ID NO: 29, preferably a CDRL2 sequence of SEQ ID NO: 29;
   a CDRL3 sequence at least 90% identical to SEQ ID NO: 30, preferably a CDRL3 sequence of SEQ ID NO: 30;
   a CDRH1 sequence at least 90% identical to SEQ ID NO: 31, preferably a CDRH1 sequence of SEQ ID NO: 31;
   a CDRH2 sequence at least 90% identical to SEQ ID NO: 32, preferably a CDRH2 sequence of SEQ ID NO: 32; and
   a CDRH3 sequence at least 90% identical to SEQ ID NO: 33, preferably a CDRH3 sequence of SEQ ID NO: 33,
and wherein the antibody or antigen binding fragment thereof binds said epitope with a dissociation rate constant (k_{off}) that is greater than the k_{off} of a non-humanised antibody or antigen binding domain thereof comprising the CDRL1, CDRL2, CDRL3, CDRH1, CDRH2, and CDRH3 sequences;
b) a transmembrane domain;
c) one or more costimulatory domains; and
d) one or more intracellular signalling domains.

In an embodiment, the non-humanised antibody or antigen binding domain thereof comprising the CDRL1, CDRL2, CDRL3, CDRH1, CDRH2, and CDRH3 sequences is a murine antibody or antigen binding fragment thereof.

In an embodiment, a CAR comprises:
a) an extracellular domain which comprises a humanised single chain variable fragment (scFv) that binds one or more epitopes on an aberrantly glycosylated MUC1 protein, wherein the scFv comprises:
   a CDRL1 sequence of SEQ ID NO: 28;
   a CDRL2 sequence of SEQ ID NO: 29;
   a CDRL3 sequence of SEQ ID NO: 30;
   a CDRH1 sequence of SEQ ID NO: 31;
   a CDRH2 sequence of SEQ ID NO: 32; and
   a CDRH3 sequence of SEQ ID NO: 33,
and wherein the antibody or antigen binding fragment thereof binds said epitope with a dissociation rate constant (k_{off}) that is greater than the k_{off} of a non-humanised antibody or antigen binding domain thereof comprising the CDRL1, CDRL2, CDRL3, CDRH1, CDRH2, and CDRH3 sequences;
b) a transmembrane domain isolated from CD8α;
c) a costimulatory domain isolated from CD137 (4-1BB); and
d) an intracellular signalling domain isolated from CD3.

In other embodiments of the invention, a CAR comprises:
(a) an extracellular domain which comprises a humanised antibody or antigen binding domain thereof that binds one or more epitopes on an aberrantly glycosylated MUC1 protein, wherein the antibody or antigen binding domain thereof binds said epitope with a dissociation rate constant (k_{off}) that is at least 1×10⁻³ s⁻¹ and has at least one of the following additional properties:
   (i) increased specificity for cells expressing aberrantly glycosylated MUC1 protein; and/or
   (ii) reduced T cell exhaustion;
(b) a transmembrane domain;
(c) one or more costimulatory domains; and
(d) one or more intracellular signalling domains.
   Increased specificity of a CAR for cells expressing aberrantly glycosylated MUC1 protein means that T cells modified to express said CAR have an increased ability to discriminate among cells expressing aberrantly glycosylated MUC1 protein and cells which do not express aberrantly glycosylated MUC1 protein. Specificity for cells expressing aberrantly glycosylated MUC1 protein can be determined using any methods known in the art, such as by measuring cytokine release (e.g., IFN-γ release) of T cells modified to express a CAR provided herein when co-cultured with cells either positive or negative for aberrantly glycosylated MUC1 protein. Cytokine release, such as IFN-γ release is a marker of T-cell activation.

CARs having a fast off-rate (i.e., higher dissociation rate constant (k_{off})) may also result in reduced T cell exhaustion in the presence of aberrantly glycosylated MUC1 protein. T cell exhaustion can be determined by any method known in the art, such as by measuring antigen independent (basal) signalling of T cells modified to express a CAR provided herein. Basal activation of CAR-T cells can be determined by measuring the level of PD1, TIM3, LAG3 markers (exhaustion markers) and interferon-gamma (IFNγ) secretion (activation marker) in the absence of the antigen.

### Polypeptide

Various polypeptides are contemplated herein, including, but not limited to, CAR polypeptides and fragments thereof, cells and compositions comprising the same, antibodies and vectors that express polypeptides. In preferred embodiments, a polypeptide comprising one or more CARs is provided. In particular embodiments, the CAR is an anti-AG-MUC1 CAR comprising an amino acid sequence as set forth in any one of SEQ ID NOs: 58, 59, 60, or 61.

“Polypeptide,” “polypeptide fragment,” “peptide” and “protein” are used interchangeably, unless specified to the contrary, and according to conventional meaning, i.e., as a sequence of amino acids. Polypeptides may be synthesized or recombinantly produced. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides comprise a signal (or leader) sequence at the N-terminal end of the protein, which co-translationally or posttranslationally directs transfer of the protein. Illustrative examples of suitable signal sequences useful in CARs contemplated herein include, but are not limited to the IgG1 heavy chain signal polypeptide, a CD8α signal polypeptide, or a human GM-CSF receptor alpha signal polypeptide. An exemplary signal sequence useful in the CARs is shown in SEQ ID NO: 8.

Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as contemplated herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences.

### Polynucleotide

In preferred embodiments, a polynucleotide encoding one or more CAR polypeptides is provided. As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides.

In various illustrative embodiments, polynucleotides include expression vectors, viral vectors, and transfer plasmids, and compositions and cells comprising the same. In various illustrative embodiments, polynucleotides encode a polypeptide contemplated herein, including, but not limited to the polypeptide sequences set forth in SEQ ID NOS: 2-7, and 10-61.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

### Vectors

The present invention provides vectors which comprise a polynucleotide encoding one or more CAR polypeptides as described herein.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids), transposons, cosmids, bacterial artificial chromosomes, and viral vectors. Useful viral vectors include, e.g., replication defective retroviruses and lentiviruses.

In particular embodiments, the vectors are expression vectors. Expression vectors may be used to produce CARs and polypeptides of the invention. In addition, expression vectors may include additional components which allow for the production of viral vectors, which in turn comprise a polynucleotide of the invention. Viral vectors may be used for delivery of the polynucleotides of the invention to a subject or a subject's cells. Examples of expression vectors include, but are not limited to, plasmids, autonomously replicating sequences, and transposable elements. Additional exemplary vectors include, without limitation, plasmids, phagemids, cosmids, transposons, artificial chromosomes such as yeast artificial chromosome (YAC), bacterial artificial chromosome (BAC), or PI-derived artificial chromosome (PAC), bacteriophages such as lambda phage or MI 3 phage, and animal viruses.

Additional examples of expression vectors are pCIneo vectors (Promega) for expression in mammalian cells; pLenti4/V5-DEST™ pLenti6/V5-DEST™ and pLenti6.2/V5-GW/lacZ (Invitrogen)) for lentivirus-mediated gene transfer and expression in mammalian cells. In particular embodiments, the coding sequences of the CARs and polypeptides disclosed herein can be ligated into such expression vectors for the expression of the CARs and/or polypeptides in mammalian cells.

In particular embodiments, the expression vectors of the present invention are BACs which comprise a polynucleotide of the invention. In particular embodiments, the BACs additionally comprise one or more polynucleotides encoding for proteins necessary to allow the production of a viral vector when expressed in a producer or packaging cell line. By way of example, PCT applications WO2017/089307 and WO2017/089308 describe expression vectors used to produce retroviral vectors, in particular lentiviral vectors. In a particular embodiment, the present invention includes the expression vectors described in WO2017/089307 and WO2017/089308, comprising a polynucleotide of the invention.

The “control elements” or “regulatory sequences” present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

### Vectors for Delivery

The present invention also provides vectors for delivery of the polynucleotides of the invention to a subject and/or subject's cells. Examples of such vectors include, but are not limited to, plasmids, autonomously replicating sequences, transposable elements, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and viral vectors.

Examples of categories of animal viruses useful as viral vectors include, without limitation, retrovirus (including lentivirus), adenovirus, adeno-associated virus (AAV), herpesvirus (e.g., herpes simplex virus), poxvirus, baculovirus, papillomavirus, and papovavirus (e.g., SV40). These vectors are referred to herein as “viral vectors”.

As the skilled person will appreciate, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer.

Retroviruses are a common tool for gene delivery (Miller, 2000, Nature. 357: 455-460). In particular embodiments, a retrovirus is used to deliver a polynucleotide encoding a CAR of the invention to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors and more particularly lentiviral vectors may be used in practicing particular embodiments. Accordingly, the term “retrovirus” or “retroviral vector”, as used herein is meant to include “lentivirus” and “lentiviral vectors” respectively.

Viral particles will typically include various viral components and sometimes also host cell components in addition to nucleic acid(s).

As stated above, the term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector,” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and US. The U3 region contains the enhancer and promoter elements. The U5 region is the sequence between the primer binding site and the R region and contains the polyadenylation sequence. The R (repeat) region is flanked by the U3 and U5 regions. The LTR comprises U3, R, and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., J Virol. 1995; 69(4): 2101-9. Several retroviral vectors use the minimal packaging signal (also referred to as the psi [W] sequence) needed for encapsidation of the viral genome. Thus, as used herein, the terms “packaging sequence,” “packaging signal,” “psi” and the symbol “W,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication defective. As used herein, the term “replication-defective” refers to virus that is not capable of complete, effective replication such that infective virions are not produced (e.g., replication-defective lentiviral progeny). The term “replication-competent” refers to wildtype virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

According to certain specific embodiments, most or all of the viral vector backbone sequences are derived from a lentivirus, e.g., HIV-I. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (Science. 1996; 272(5259): 263-7; Proc Natl Acad Sci USA. 1996; 93(21): 11382-8; Curr Opin Biotechnol. 1998; 9(5): 457-63); Zufferey al., Nat Biotechnol. 1997; 15(9): 871-5; Dull et al., J Virol. 1998; 72(11): 8463-71; U.S. Pat. Nos. 6,013,516; and 5,994,136, many of which may be adapted to produce a viral vector or transfer plasmid.

In various embodiments, vectors comprise a promoter operably linked to a polynucleotide encoding a CAR polypeptide.

In particular embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

### Control Elements

In particular embodiments, vectors, including but not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” ‘cell type specific,” ‘cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukaemia virus (MoN4LV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (βKIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer binding site substituted (MND) promoter (Challita et al., J Virol. 69(2)•748-55 (1995)).

In one embodiment, a vector comprises an PGK promoter.

In a particular embodiment, it may be desirable to express a polynucleotide comprising a CAR from a T cell specific promoter.

As used herein, “conditional expression” may refer to any type of conditional expression including, but not limited to, inducible expression: repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-I promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, a polynucleotide or cell comprising the polynucleotide utilizes a suicide gene, including an inducible suicide gene to reduce the risk of direct toxicity and/or uncontrolled proliferation. In specific embodiments, the suicide gene is not immunogenic to the host comprising the polynucleotide or cell. A certain example of a suicide gene that may be used is caspase-9 or caspase-8 or cytosine deaminase. Caspase-9 can be activated using a specific chemical inducer of dimerization (CID).

In certain embodiments, vectors comprise gene segments that cause the immune effector cells, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound.

Negative selectable genes are known in the art, and include, inter alia the following: the Herpes simplex virus type I thymidine kinase (HSV-I TK) gene (Wigler et al., Cell I: 223, 1977) which confers ganciclovir sensitivity; the cellular hypoxanthine phosphoribosyltransferase (HPRT) gene, the cellular adenine phosphoribosyltransferase (APRT) gene, and bacterial cytosine deaminase, (Mullen et al., Proc. Natl. Acad. Sci. USA 89.33 (1992)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. And Cell. Biology 11:3374-3378, 1991. In addition, in preferred embodiments, the polynucleotides encoding the CARS are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra.

### Vector Production

In particular embodiments, a cell (e.g., an immune effector cell) is transduced with a retroviral vector, e.g., a lentiviral vector, encoding a CAR. For example, an immune effector cell is transduced with a vector encoding a CAR of the present invention. These transduced cells can elicit a CAR-mediated cytotoxic response.

A “host cell” includes cells electroporated, transfected, infected, or transduced in vivo, ex vivo, or in vitro with a vector or a polynucleotide. Host cells may include packaging cells, producer cells, and cells transduced with viral vectors. In particular embodiments, host cells transduced with viral vectors are administered to a subject in need of therapy. In certain embodiments, the term “target cell” is used interchangeably with host cell and refers to transfected, infected, or transduced cells of a desired cell type. In preferred embodiments, the target cell is a T cell.

Large scale viral vector production is often necessary to achieve a suitable viral titre. Viral particles may be produced by transfecting a transfer vector into a packaging cell line that comprises viral structural and/or accessory genes, e.g., gag, POI, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. In particular embodiments, a retroviral/lentiviral transfer vector is introduced into a packaging cell line, via transfection, transduction or infection, to generate a producer cell or cell line. In particular embodiments, packaging vectors are introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

As used herein, the term “packaging cell lines” is used in reference to cell lines that do not contain a packaging signal but do stably or transiently express viral structural proteins and replication enzymes (e.g., gag, pol and env) which are necessary for the correct packaging of viral particles. Any suitable cell line can be employed to prepare packaging cells. Generally, the cells are mammalian cells. In a particular embodiment, the cells used to produce the packaging cell line are human cells. Suitable cell lines which can be used include, for example, CHO cells, BHK cells, NOCK cells, C3H IOT1/2 cells, FLY cells, Psi2 cells, BOSC 23 cells, PA317 cells, WEHI cells, COS cells, BSC 1 cells, BSC 40 cells, BMT 10 cells, VERO cells, W 138 cells, MRC5 cells, A549 cells, HT1080 cells, HEK293 cells, HEK293T cells, B-50 cells, 3T3 cells, NIH3T3 cells, HepG2 cells, Saos-2 cells, Huh7 cells, HeLa cells, W 163 cells, 211 cells, and 21 IA cells. In preferred embodiments, the packaging cells are HEKK293 cells or HEK293 T cells. In another preferred embodiment, the cells are HEK293T cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Producer cell line includes those cell lines described in WO2017/089307 and WO2017/089308, which comprise all of the elements which are necessary for the production of a retroviral vector, in a single locus in the host cell genome.

Methods of preparing viral stock solutions are known in the art and are illustrated by, e.g., Y. Soneoka et al. (1995) Nucl. Acids Res. 23:628-633, and N. R. Landau al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G protein (VSV-G) envelope proteins, which allows HIV to infect a wider range of cells because HIV envelope proteins (encoded by the env gene) normally target the virus to CD4+ presenting cells. In a preferred embodiment, lentiviral envelope proteins are pseudotyped with VSV-G. In one embodiment, packaging cells produce a recombinant retrovirus, e.g., lentivirus, pseudotyped with the VSV-G envelope glycoprotein.

In other embodiments, viral vectors may be pseudotyped with an envelope protein from either another retrovirus or an unrelated virus. The skilled person will appreciate that the viral vectors of the invention may be pseudotyped with any suitable envelope protein.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

### Immune Effector Cell

In various embodiments, cells genetically modified to express the CARs contemplated herein, for use in the treatment of cancer are provided. As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the CARs contemplated by the present invention are introduced and expressed in immune effector cells so as to redirect their specificity to a target antigen of interest, e.g., an AG-MUC1 protein.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8+ T cells), tumor infiltrating lymphocytes (TILs), and helper T cells (HTLs; CD4+ T cells. In one embodiment, immune effector cells include natural killer (NK) cells. In one embodiment, immune effector cells include natural killer T cells. In one embodiment, the immune effector cells include cytotoxic T lymphocytes.

Immune effector cells can be autologous/autogeneic (“self”) or non-autologous (“nonself,” e.g., allogeneic, syngeneic or xenogeneic).

“Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison.

In certain embodiments, the cells are allogeneic.

In certain embodiments, the cells are autologous.

Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper I (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺ CD8⁺ T cell, CD4⁻ CD8⁻ T cell, or any other subset of T cells. Other illustrative populations of T cells suitable for use in particular embodiments include naive T cells and memory T cells.

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34 population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain an AG-MUC1 specific CAR may be referred to as “AG-specific redirected immune effector cells.”

Methods for making the immune effector cells which express the CARs of the present invention are provided in particular embodiments. In one embodiment, the method comprises transfecting or transducing immune effector cells isolated from an individual such that the immune effector cells express one or more CARs of the invention. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells described herein, the immune effector cells are obtained from a subject. In particular embodiments, the CAR-modified immune effector cells comprise T cells.

In particular embodiments, PBMC may be directly genetically modified to express CARs using methods contemplated herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the CARs of the invention (e.g., transduced with a viral vector comprising a nucleic acid encoding a CAR) and then are activated and expanded in vitro. In various embodiments, T cells can be activated and expanded before or after genetic modification to express a CAR.

In particular embodiments, a population of modified immune effector cells for the treatment of cancer comprises a CAR as disclosed herein. For example, a population of modified immune effector cells are prepared from peripheral blood mononuclear cells (PBMCs) obtained from a patient diagnosed with cancer (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. A vector carrying the coding sequence of a CAR of the invention can be introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and/or anti-CD28 antibodies and IL-2 or any other methods known in the art as described elsewhere herein.

Standard procedures are used for cryopreservation of T cells expressing the CAR protein T cells for storage and/or preparation for use in a human subject.

In a further embodiment, a mixture of, e.g., one, two, three, four, five or more, different vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

### T Cell Manufacturing Methods

Methods of manufacturing T cells for human therapy are known in the art. In preferred embodiments, the T cells manufactured by the methods contemplated herein provide improved adoptive immunotherapy compositions. Without wishing to be bound to any particular theory, it is believed that the T cell compositions manufactured by the methods in particular embodiments contemplated herein are imbued with superior properties, including increased survival, expansion in the relative absence of differentiation, persistence in vivo and superior anti-exhaustion properties. T cells modified to express an anti-AG-MUC1 CAR exhibit a lower binding kinetic to the AG-MUC1 target do have a lower potential to exhaust in vivo in the presence of the AG-MUC1 target. Low tendency towards exhaustion of anti-AG-MUC1 CAR-T cells retains their effector function, leads to a low sustained expression of inhibitory receptors and retains a transcriptional state of a functional effector or memory T cells. Exhaustion is not a desired feature of a CAR-T therapy since it prevents optimal control of infection and tumors.

In one embodiment, the T cells are modified by transducing the T cells with a viral vector comprising an anti-AG-MUC1 CAR contemplated herein. Anti-AG-MUC1 CAR-T cells show low levels of basal CAR activation and interferon-gamma (IFNγ) secretion in the absence of the antigen which is a desired attribute of a CAR-T therapy. The propensity of a CAR to antigen-independent (basal) signalling might indicate a self-aggregation leading to antigen-independent CAR activation that in turn could cause early CAR exhaustion resulting in loss of therapeutic potency (Ajina and Maher, 2018; Long et al., 2015a). Basal activation of CAR-T cells is determined through the level of PD1, TIM3, LAG3 markers and CAR-T ability to secret IFNγ in the absence of antigen. Humanised anti-AG-MUC1 CAR-T cells with a low binding kinetic showed low IFNγ secretion and retained a memory phenotype in vitro when compared to un-transduced matching donor T-cells.

In one embodiment, the T cells are modified by transducing the T cells with a viral vector comprising an anti-AG-Muc1 CAR contemplated herein that requires a higher target threshold to be activated rendering it a ‘safer’ CAR. It has been shown that CARs with high affinity can lead to collateral targeting of healthy tissues resulting in on/off-target, off-tumour toxicity (Johnson et al., 2015; Park et al., 2017; Watanabe et al., 2018). Therefore, we compared slow off-rate and fast off-rate CAR Ts on their ability to discriminate between Tn-MUC1-positive and -negative cells. Both fast and slow off-rate CAR Ts secreted IFNγ in response to Tn-MUC1-positive cell lines, however slow off-rate CAR Ts produced IFNγ in response to tumour cells lacking the MUC1 protein, whereas fast off-rate CAR Ts did not. These data suggest that fast off-rate anti-AG-Muc1 CAR T cells specifically recognise Tn-MUC1 target, minimizing the risk of off-target:off-tumour toxicities in patients in addition to their anti-exhaustion properties.

### Pharmaceutical Composition

Immune effector cells described herein may be incorporated into pharmaceutical compositions for use in the treatment of the human diseases described herein. In one embodiment, the pharmaceutical composition comprises an immune effector cell optionally in combination with one or more pharmaceutically acceptable carriers and/or excipients.

Such compositions comprise a pharmaceutically acceptable carrier as known and called for by acceptable pharmaceutical practice, see e.g. Remington's Pharmaceutical Sciences, 16th edition (1980) Mack Publishing Co.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for intravenous administration.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumour size, extent of infection or metastasis, and condition of the patient (subject). It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a litre or less, can be 500 mls or less, even 250 mls or 100 mls or less. Hence the density of the desired cells is typically greater than 10⁶ cells/ml, e.g. greater than 10⁶, 10⁷, 10⁸ or 10⁹ cells/ml.

The clinically relevant number of immune cells can be apportioned into multiple infusions that cumulatively equal or exceed 10⁵, 10⁶, 10⁷, 10⁸ 10⁹, 10¹⁰, 10¹¹, or 10¹² cells. In some embodiments, particularly since all the infused cells will be redirected to a particular target antigen, lower numbers of cells, in the range of 10⁶/kilogram (10⁶ to 10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFNγ, IL-2, IL-12, TNFα, IL-18, and TNFβ, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular, compositions comprising the CAR-modified T cells contemplated herein are used in the treatment of cancer. In particular embodiments, CAR-modified T cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In particular embodiments, compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

In another preferred embodiment, compositions comprising T cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a particular embodiment, compositions comprise an effective amount of CAR expressing immune effector cells, alone or in combination with one or more therapeutic agents. Thus, the CAR expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising CAR-expressing immune effector cells disclosed herein may be administered in conjunction with any number of chemotherapeutic agents which are known in the art.

A variety of other therapeutic agents may be used in conjunction with the compositions described herein. In one embodiment, the composition comprising CAR expressing immune effector cells is administered with an anti-inflammatory agent. Anti-inflammatory agents or drugs are known in the art.

In certain embodiments, the compositions described herein are administered in conjunction with a cytokine. By “cytokine” as used herein is meant a generic term for proteins released by one cell population that act on another cell as intercellular mediators. Examples of such cytokines are known in the art.

The pharmaceutical composition may be included in a kit of parts of containing the CAR expressing immune effector cell together with other medicaments, optionally and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have been diagnosed with, or are at risk for having a cancer that expresses an AG-MUC1 protein.

As used herein, the term “patient” refers to a subject that has been diagnosed with a particular disease, disorder, or condition that can be treated with the gene therapy vectors, cell-based therapeutics, and methods disclosed elsewhere herein.

As used herein, the terms “treatment,” “treating,” and derivatives thereof is meant therapeutic therapy, and includes any beneficial or desirable effect on the symptoms or pathology of a disease or pathological condition and may include even minimal reductions in one or more measurable markers of the disease or condition being treated. Treatment can involve optionally either the reduction of the disease or condition, or the delaying of the progression of the disease or condition, e.g., delaying tumour outgrowth. “Treatment” does not necessarily indicate complete eradication or cure of the disease or condition, or associated symptoms thereof. In reference to a particular condition, treating means: (1) to ameliorate the condition or one or more of the biological manifestations of the condition (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; or (4) to slow the progression of the condition or one or more of the biological manifestations of the condition.

As used herein, “prevention” means the prophylactic administration of a drug, such as an agent, to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

### Cancer

As used herein, the terms “cancer”, “neoplasm”, and “tumour”, are used interchangeably and in either the singular or plural form, refer to cells that have undergone a malignant transformation or undergone cellular changes that result in aberrant or unregulated growth or hyperproliferation. Such changes or malignant transformations usually make such cells pathological to the host organism, thus precancers or pre-cancerous cells that are or could become pathological and require or could benefit from intervention are also intended to be included. Primary cancer cells (that is, cells obtained from near the site of malignant transformation) can be readily distinguished from non-cancerous cells by well-established techniques, such as histological examination.

The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumours of any stage, including what a clinician refers to as precancer, tumours, in situ growths, as well as late stage metastatic growths.

As used herein, the term “malignant” refers to a cancer in which a group of tumour cells display one or more of uncontrolled growth (i.e., division beyond normal limits), invasion (i.e., intrusion on and destruction of adjacent tissues), and metastasis (i.e., spread to other locations in the body via lymph or blood).

A “cancer cell” refers to an individual cell of a cancerous growth or tissue. Cancer cells include both solid cancers and liquid cancers. A “tumour” or “tumour cell” refers generally to a swelling or lesion formed by an abnormal growth of cells, which may be benign, pre-malignant, or malignant. Most cancers form tumours, but liquid cancers, e.g., leukaemia, do not necessarily form tumours. For those cancers that form tumours. the terms cancer (cell) and tumour (cell) are used interchangeably. The amount of a tumour in an individual is the “tumour burden” which can be measured as the number, volume, or weight of the tumour.

In one embodiment, the target cell expresses an antigen, e.g., a target antigen that is not substantially found on the surface of other normal (desired) cells.

In one embodiment, the target cell is a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In one embodiment, the target cell expresses an AG-MUC1 protein. In one embodiment, the target cell is a hematopoietic cell, an oesophageal cell, a lung cell, an ovarian cell, a cervix cell, a pancreatic cell, a cell of the gall bladder or bile duct, a stomach cell, a colon cell, a breast cell, a goblet cell, an enterocyte, a stem cell, an endothelial cell, an epithelial cell, or any cell that express an AG-MUC1 protein.

In one embodiment, the target cell is solid cancer cell that expresses an AG-MUC1 protein.

Illustrative examples of cells that can be targeted by the compositions and methods contemplated in particular embodiments include, but are not limited to those of the following solid cancers: adrenal cancer, adrenocortical carcinoma, anal cancer, appendix cancer, astrocytoma, atypical teratoid/rhabdoid tumour, basal cell carcinoma, bile duct cancer, bladder cancer, bone cancer, brain/CNS cancer, breast cancer, bronchial tumours, cardiac tumours, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumour, extragonadal germ cell tumour, eye cancer, fallopian tube cancer, fibrous histiosarcoma, fibrosarcoma, gallbladder cancer, gastric cancer, gastrointestinal carcinoid tumours, gastrointestinal stromal tumour (GIST), germ cell tumours, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumour, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumours, papillary carcinoma, paraganglioma, parathyroid cancer, penile cancer, pharyngeal cancer, pheochromocytoma, pinealoma, pituitary tumour, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumour.

In another embodiment, the cell is a solid cancer cell that expresses an AG-MUC1 protein. Exemplary AG-MUC1 expressing solid cancer cells that may be prevented, treated, or ameliorated with the compositions include, but are not limited to: oesophageal cancer, lung cancer, ovarian cancer, cervical cancer, pancreatic cancer, cholangiocarcinoma, gastric cancer, colon cancer, bladder cancer, kidney cancer, and breast cancer.

In a particular embodiment, the target cell is a liquid cancer or haematological cancer cell that expresses an AG-MUC1 protein.

Illustrative examples of liquid cancers or haematological cancers that may be prevented, treated, or ameliorated with the compositions contemplated in particular embodiments include, but are not limited to: leukaemias, lymphomas, and multiple myeloma. Illustrative examples of cells that can be targeted by anti-STN CARs contemplated in particular embodiments include, but are not limited to those of the following leukaemias: acute lymphocytic leukaemia (ALL), T cell acute lymphoblastic leukaemia, acute myeloid leukaemia (AML), myeloblastic, promyelocytic, myelomonocytic, monocytic, erythroleukemia, hairy cell leukaemia (HCL), chronic lymphocytic leukaemia (CLL), and chronic myeloid leukaemia (CML), chronic myelomonocytic leukaemia (CNNL) and polycythemia vera.

### Method of Increasing Cytotoxicity

The genetically modified immune effector cells contemplated herein provide improved methods of adoptive immunotherapy for use in the prevention, treatment, and amelioration cancers that express AG-MUC1 proteins, or for preventing, treating, or ameliorating at least one symptom associated with an AG-MUC1 protein expressing cancer.

In various embodiments, the genetically modified immune effector cells contemplated herein provide improved methods of adoptive immunotherapy for use in increasing the cytotoxicity in cancer cells that express an AG-MUC1 protein in a subject or for use in decreasing the number of cancer cells expressing AG-MUC1 in a subject.

In particular embodiments, the specificity of a primary immune effector cell is redirected to cells expressing AG-MUC1 protein, e.g., cancer cells, by genetically modifying the primary immune effector cell with a CAR contemplated herein. In various embodiments, a viral vector is used to genetically modify an immune effector cell with a particular polynucleotide encoding a CAR comprising an anti-AG-MUC1 antibody or antigen binding domain that binds an AG-MUC1; a hinge domain; a transmembrane (TM) domain, and one or more co-stimulatory domains; and one or more intracellular signalling domains.

In one embodiment, a type of cellular therapy where T cells are genetically modified to express a CAR that targets an AG-MUC1 expressing protein expressed on cancer cells, and the CAR T cell is infused to a recipient in need thereof is provided. The infused cell is able to kill disease causing cells in the recipient. Unlike antibody therapies, CAR T cells are able to replicate in vivo resulting in long-term persistence that can lead to sustained cancer therapy.

In one embodiment, the CAR T cells can undergo robust in vivoT cell expansion and can persist for an extended amount of time. In another embodiment, the CAR T cells evolve into specific memory T cells that can be reactivated to inhibit any additional tumour formation or growth.

In particular embodiments, compositions comprising immune effector cells comprising the CARs contemplated herein are used in the treatment of conditions associated with cancer cells or cancer stem cells that express AG-MUC1 proteins.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., a genetically modified T cell or vector encoding a CAR, to produce, elicit, or cause a greater physiological response (i.e., downstream effects) compared to the response caused by a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30, 50, 100, 200, 500 or more times the response produced by a control composition.

By “decrease” or “lower,” or “lessen,” or “reduce,” or “abate” refers generally to the ability of a composition contemplated herein to produce, elicit, or cause a lesser physiological response (i.e., downstream effects) compared to the response caused by a control molecule/composition. A “decreased” or “reduced” amount is typically a “statistically significant” amount, and may include a decrease that is 1.1, 1.2, 1.5, 2, 3, 4, 5, 6, 7, 8, 9, 10, 15, 20, 30, 50, 100, 200, 500 or more times the response (reference response) produced by a control composition, or the response in a particular cell lineage.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

One of ordinary skill in the art would recognize that multiple administrations of the compositions contemplated herein may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & sons, NY, N.Y.)

In the case of T cell-mediated killing, CAR-ligand binding initiates CAR signalling to the T cell, resulting in activation of a variety of T cell signalling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of proinflammatory cytokines that can induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

The present inventors generated a scFv (target binding moiety of the CAR) by humanising the VL and VH immunoglobulin domains derived from the murine-originated mAb 5E5 (Sørensen et al., 2006). Humanisation of VL and VH was performed in scFv format. The humanised scFv retained specificity to Tn/STn-MUC1 peptides in Biacore assays albeit with a different kinetic profile compared to the fully murine scFv. Engineered human T-cells expressing a CAR containing the humanised Tn/STn-MUC1-specific scFv were employed to generate key data showing in vitro potency and specificity of the humanised Tn-MUC1 CAR Ts.

### Example 1—Generation of Binding Domain

High-affinity glycopeptide-specific antibodies have been developed to target TnMUC1 (Sørensen et al., 2006) (Tarp et al., 2007). Mouse 5E5 mAb binds with an affinity of 1.7 nM and can lyse breast cancer cells via complement mediated and antibody-dependent cellular cytotoxicity (Lavrsen et al., 2013). TnMUC1 specific CAR-T cells, which comprise the variable domains of the 5E5 mAb, can eliminate pancreatic and leukemia in xenograft models, and, similar to the original antibody, display cancer-specificity and negligible reactivity against normal tissues (Posey et al., 2016).

In silico methods were used to produce humanised variants of murine 5E5 VH and VL and to clone a set of humanised scFv constructs for subsequent protein expression and target binding characterisation. Furthermore, the study aimed to predict immunogenicity risk for each scFv by considering the framework templates and restored murine amino acids (back-mutations) used during humanisation.

### Methods

Design and Molecular Cloning of Murine scFv Expression Constructs

Amino acid sequences of murine 5E5 VH and VL (Table 2—Kabat-defined CDR residues are underlined) were combined in VL-VH and VH-VL orientations to generate amino acid sequences for two scFv molecules. scFv sequences comprised a (G₄S)₃ linker between VL and VH chains, an N-terminal Campath signal peptide (MGWSCIILFLVATATGVHS) and either a C-terminal hexa-His tag (HHHHHH, VL-VH scFv) or a C-terminal hepta-His tag (HHHHHHH, VH-VL scFv).

TABLE 2
Amino acid sequences of VL and VH Ig domains
from murine mAb 5E5

| | |
|---|---|
| VL | ELVMTQSPSSLTVTAGEKVTMICKSSQSLLNSGDQKNYLTWYQQKP |
| | GQPPKLLIFWASTRESGVPDRFTGSGSGTDFTLTISSVQAEDLAVY |
| | YCQNDYSYPLTFGAGTKLELK (SEQ ID NO: 62) |
| VH | QVQLQQSDAELVKPGSSVKISCKASGYTFTDHAIHWVKOKPEQGLE |
| | WIGHFSPGNTDIKYNDKFKGKATLTVDRSSSTAYMQLNSLTSEDSA |
| | VYFCKTSTFFFDYWGQGTTLTVSS (SEQ ID NO: 63) |

Codon optimised DNA sequence was generated using software Leto 1.0.26 (Entelechon GmbH). These were then modified to include a 5′ adaptor, containing restriction endonuclease site HindIII followed by a Kozak sequence, and a 3′ adaptor, containing tandem STOP codons and restriction endonuclease site EcoRI. DNA sequences were synthesised by Integrated DNA Technologies (IDT) Inc. as double-stranded fragments (gBlocks).

pTT5 backbone in which the multiple cloning site has been modified and gBlocks were digested with HindIII and EcoRI restriction endonucleases. T4 DNA ligase was used to ligate cut vector backbone and cut gBlock insert. Ligation mixtures were transformed into NEB 5-alpha competent E. coli (subcloning efficiency) and positive transformants selected on plates of LB agar supplemented with 100 ug/ml carbenicillin and 1% w/v glucose. Colonies for putative clones were cultured, plasmid DNA extracted, and DNA subjected to Sanger sequencing to identify correct clones.

In Silico Generation of Amino Acid Sequences for Humanised VH and VL Chains Derived from Murine mAb 5E5

Amino acid sequence encompassing the 3 complementarity determining regions (CDRs) within the VL and VH domains of murine mAb 5E5 were identified (Table 2). From these, VH and VL sequences were generated in which the Kabat-defined CDRs (Kabat et al., 1991) were masked (replaced by one-letter amino acid symbol X denoting unspecified amino acid) (Table 3—masked residues are underlined). These four sequences were used as input for the Basic Local Alignment Search Tool (BLAST) algorithm (Altschul et al., 1997) to identify similar frameworks from human V gene (heavy, kappa, lambda) germline databases. In addition, framework 4 amino acid sequence from 5E5 VH and VL were used to identify similar human J gene segments.

TABLE 3
Amino acid sequence of VL and VH Ig domains from
murine mAb 5E5 in which CDRs have been masked

| | |
|---|---|
| VL | ELVMTQSPSSLTVTAGEKVTMICXXXXXXXXXXXXXXXXXWYQQKP |
| | GQPPKLLIFXXXXXXXGVPDRFTGSGSGTDFTLTISSVQAEDLAVY |
| | YCXXXXXXXXXFGAGTKLELK (SEQ ID NO: 64) |
| VH | QVQLQQSDAELVKPGSSVKISCKASGYTFTXXXXXWVKQKPEQGLE |
| | WIGXXXXXXXXXXXXXXXXXKATLTVDRSSSTAYMQLNSLTSEDSA |
| | VYFCKTXXXXXXXWGQGTTLTVSS (SEQ ID NO: 65) |

Human V and J gene segments were chosen as template frameworks based on their identity to 5E5 sequence, in-house analysis of individual and pairing frequency of V genes and previous experience of the use of particular templates for legacy humanisation.

The chosen human V gene frameworks were compared to the respective murine VH and VL sequences to identify potential sites (Kabat numbering) that could undergo back-mutation to the corresponding mouse amino acid at that position. In-house collated evidences rules for the importance of certain framework positions in the likely maintenance of CDR conformation (and antigen binding affinity) were used to identify back-mutations considered most significant (primary mutations) and those of lower significance (secondary mutations). The extent of spatial clustering of the identified back-mutations was examined by building a 3D homology model of the mouse 5E5 Fv domain using software Chemical Computing Group (CCG) Molecular Operating Environment (MOE) 2016.0802. Initial humanised VH and VL sequences (chains H0 and L0) were generated by constructing a straight graft of the mouse CDRs into the chosen human germline templates. The apparent spatial clustering of back-mutation sites was used to reduce the potential number of variants of back-mutation containing humanised chains by introducing spatially-clustered mutations simultaneously.

In all, 6 humanised VH amino acid sequences (H0-H5) and 4 humanised VL amino acid sequences (L0-L3) were generated.

Design and Molecular Cloning of Humanised scFv Expression Constructs

Amino acid sequences of humanised VH and VL chains were systematically combined in the VL-VH orientation to generate amino acid sequences for 24 scFv molecules. In addition, a single scFv comprised of the L3 and H3 chains in the VH-VL orientation was also assembled. scFv sequences comprised a (G₄S)₄ linker between VL and VH chains, an N-terminal Campath signal peptide (MGWSCIILFLVATATGVHS) and either a C-terminal hexa-His tag (AAAHHHHHH, VL-VH scFvs) or a C-terminal hepta-His tag (AAAHHHHHHH, H3-L3 scFv). scFv protein sequences were reverse translated and codon optimised using software Leto 1.0.26 (Entelechon GmbH).

In addition, two control α-TnMUC1 scFv molecules were designed using information contained in patent application WO2015/159076. The VH and VL amino acid sequences of humanised antibodies Ab1 and Ab2 (shown to have comparable binding to TnMUC1 as 5E5) were subjected to the same process as described above generating codon-optimised DNA sequence for two scFv molecules derived from Ab1 and Ab2.

All codon optimised DNA sequences were modified to include 5′ and 3′ adaptors and synthesised as described above.

Molecular cloning of expression constructs was completed as described above except that during the ligation step either, as per standard methods one insert was used per ligation reaction, or a multiplex approach was used, where up to 6 inserts were included in a single ligation reaction. An ˜3:1 molar ratio of individual insert to vector was maintained.

### Immunogenicity Risk Prediction

The amino acid sequences of humanised scFv molecules and VL-VH murine scFv (with Campath signal peptide and the C-terminal His tag removed) were used for immunogenicity risk prediction. The two humanised control scFvs derived from WO2015/159076 were excluded from this process. T cell epitopes were predicted across scFv sequences using TEpredict (Antonets, 2010) at a percentile threshold of 3 with multiple MHC class II allotype matrices (Singh, 2001). Predicted 9-mer peptide epitope sequences were scanned against databases of human antibody germline sequences and tregitope sequences (De Groot, 2008), using an in-house perl script, and any peptides matching these sequences were removed. Immunogenicity risk scores were generated by summing the number of remaining T-cell epitopes. Scores were ranked against similar scores for a test set of antibodies with a known immunogenicity response in a clinical setting.

### Results

### Humanisation of 5E5 VH and VL

Six humanised VH chains (designated H0, H1, H2, H3, H4 and H5) were generated (Table 4) based on a framework consisting of IGHV1-69 (human germline immunoglobulin heavy chain IGHV1-69 V gene) joined to IGHJ6 (human germline immunoglobulin heavy chain IGHJ6 J gene). Four humanised VL chains (designated L0, L1, L2 and L3) were generated (Table 5) based on a framework consisting of IGKV4-1 (human germline immunoglobulin kappa light chain IGKV4-1 (B3) V gene) joined to IGKJ2 (human germline immunoglobulin kappa light chain IGKJ2 J gene).

TABLE 4
Humanised VH chains H0 to H5

| | |
|---|---|
| H0 | 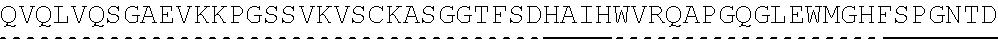 |
| | 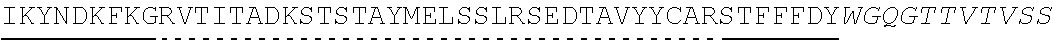 |
| | (SEQ ID NO: 66) |
| H1 | QVQLVQSGAEVKKPGSSVKVSCKASGYTFTDHAIHWVRQAPGQGLEWMGHFSPGNTD |
| | IKYNDKFKGRVTITADKSTSTAYMELSSLRSEDTAVYYCARSTFFFDYWGQGTTVTVSS |
| | (SEQ ID NO: 67) |
| H2 | QVQLVQSGAEVKKPGSSVKVSCKASGYTFTDHAIHWVRQAPGQGLEW [symbol] GHFSPGNTDI |
| | KYNDKFKGR [symbol] T [symbol] T [symbol] D [symbol] STSTAYMELSSLRSEDTAVYYCARSTFFFDYWGQGTTVTVSS |
| | (SEQ ID NO: 68) |
| H3 | QVQLVQSGAEVKKPGSSVKVSCKASGYTFTDHAIHWVRQAPGQGLEWMGHFSPGNTD |
| | IKYNDKFKGRVTITADKSTSTAYMELSSLRSEDTAVYYC [symbol] STFFFDYWGQGTTVTVSS |
| | (SEQ ID NO: 69) |
| H4 | QVQLVQSGAEVKKPGSSVKVSCKASGYTFTDHAIHWVRQAPGQGLEWMGHFSPGNTD |
| | IKYNDKFKGRVTITADKSTSTAYMELSSLRSEDTAVY [symbol] CARSTFFFDYWGQGTTVTVSS |
| | (SEQ ID NO: 70) |
| H5 | QVQLVQSGAEVKKPGSSVKVSCKASGYTFTDHAIHWVRQAPGQGLEW [symbol] GHFSPGNTDI |
| | KYNDKFKGR [symbol] T [symbol] T [symbol] D [symbol] STSTAYMELSSLRSEDTAVY [symbol] CKTSTFFFDYWGQGTTVTVSS |
| | (SEQ ID NO: 71) |

The amino acid sequences of the six humanised heavy chains H0 to H5. Amino acids belonging to human V gene IGHV1-69 are indicated with dotted underline (shown for H0 only). Amino acids belonging to human J gene IGHJ6 are shown in italic typeface (shown for H0 only). Murine Kabat-defined CDRs are underlined. Primary back-mutation clusters are bold, underlined, and in italic typeface. Two back-mutations (Kabat 27 and 30) that form part of the extended “Chothia-defined” residues of CDRH1 (Chothia et al., 1989) are bold and are present in all humanised heavy chain variants except for H0.

TABLE 5
Humanised VL chains L0 to L3

| | |
|---|---|
| L0 | 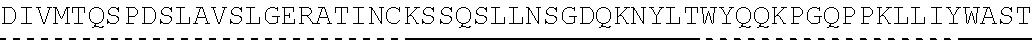 |
| | 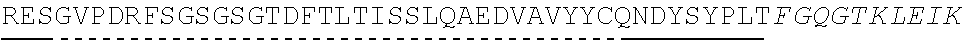 |
| | (SEQ ID NO: 72) |
| L1 | E [symbol] VMTQSPDSLAVSLGERATINCKSSQSLLNSGDQKNYLTWYQQKPGQPPKLLIYWAST |
| | RESGVPDRFSGSGSGTDFTLTISSLQAEDVAVYYCQNDYSYPLTFGQGTKLEIK |
| | (SEQ ID NO: 73) |
| L2 | DIVMTQSPDSLAVSLGERATINCKSSQSLLNSGDQKNYLTWYQQKPGQPPKLLI [symbol] WAST |
| | RESGVPDRFSGSGSGTDFTLTISSLQAEDVAVYYCQNDYSYPLTFGQGTKLEIK |
| | (SEQ ID NO: 74) |
| L3 | E [symbol] VMTQSPDSLAVSLGERATINCKSSQSLLNSGDQKNYLTWYQQKPGQPPKLLI [symbol] WAST |
| | RESGVPDRFSGSGSGTDFTLTISSLQAEDVAVYYCQNDYSYPLTFGQGTKLEIK |
| | (SEQ ID NO: 75) |

The amino acid sequences of the four humanised light chains L0 to L3. Amino acids belonging to human V gene IGKV4-1 are indicated with dotted underline (shown for L0 only). Amino acids belonging to human J gene IGKJ2 are shown in italic typeface (shown for L0 only). Murine Kabat-defined CDRs are underlined. Primary back-mutations are bold, underlined, and in italic typeface with a sole secondary back-mutation double underlined, and in italic typeface.

All ten humanised chains retained the complete murine CMRs found in the respective 5E5 VH and VL chains. L0 and H0 contained no back-mutations with all other chains containing differing combinations of the identified back-mutation clusters (Table 6).

TABLE 6
Back-mutations employed

| Humanised | Amino acid position | |
|---|---|---|
| chain | (Kabat numbering) | Back-mutation(s) present |
| L0 | — | None |
| L1 | Kabat 1 and 2 | Asp1Glu, Ile2Leu |
| L2 | Kabat 49 | Tyr49Phe |
| L3 | Kabat 1, 2, 49 | Asp1Glu, Ile2Leu, Tyr49Phe |
| H0 | — | None |
| H1 | Kabat 27 and 30 | Gly27Tyr, Ser30Thr |
| H2 | Kabat 27, 30, 48, 67, | Gly27Tyr, Ser30Thr, Met48Ile, |
| | 69, 71, 73 | Val67Ala, Ile69Leu, Ala71Val, |
| | | Lys73Arg |
| H3 | Kabat 27, 30, 93 and | Gly27Tyr, Ser30Thr, Ala93Lys, |
| | 94 | Arg94Thr |
| H4 | Kabat 27, 30 and 91 | Gly27Tyr, Ser30Thr, Tyr91Phe |
| H5 | Kabat 27, 30, 48, 67, | Gly27Tyr, Ser30Thr, Met48Ile, |
| | 69, 71, 73, 91, 93, 94 | Val67Ala, Ile69Leu, Ala71Val, |
| | | Lys73Arg, Tyr91Phe, Ala93Lys, |
| | | Arg94Thr |

### Expression Constructs

Both murine scFv constructs (VL-VH and VH-VL) were cloned and sequence verified. Of the 25 in-house humanised scFv expression constructs, 24 were cloned and sequence verified. The construct encoding L0-H0 scFv was not cloned. Both patent application-derived humanised control scFv constructs were cloned and sequence verified.

The heterologous expression of in-house humanised scFvs and control scFvs in HEK293 6E cells and the characterisation of binding of secreted scFv present in cell culture supernatant to MUC1 and TnMUC1 peptides is described below. From this work, six of the in-house humanised molecules were selected for further work along with one of the control molecules. The amino acid sequences of the selected in-house humanised scFvs are detailed in Table 7.

TABLE 7
Amino acid sequences of the six selected in-house humanised scFvs

| | |
|---|---|
| L0-H3 | DIVMTQSPDSLAVSLGERATINCKSSQSLLNSGDQKNYLTWYQQKPGQPPKLLIYWAST |
| | RESGVPDRFSGSGSGTDFTLTISSLQAEDVAVYYCQNDYSYPLTFGQGTKLEIKGGGGSGGGGSG |
| | GGGSGGGGSQVQLVQSGAEVKKPGSSVKVSCKASGYTFTDHAIHWVRQAPGQGLEWMGHFSPG |
| | NTDIKYNDKFKGRVTITADKSTSTAYMELSSLRSEDTAVYYCKTSTFFFDYWGQGTTVTVSS |
| | (SEQ ID NO: 90) |
| L0-H5 | DIVMTQSPDSLAVSLGERATINCKSSQSLLNSGDQKNYLTWYQQKPGQPPKLLIYWAST |
| | RESGVPDRFSGSGSGTDFTLTISSLQAEDVAVYYCQNDYSYPLTFGQGTKLEIKGGGGSGGGGSG |
| | GGGSGGGGSQVQLVQSGAEVKKPGSSVKVSCKASGYTFTDHAIHWVRQAPGQGLEWIGHFSPGN |
| | TDIKYNDKFKGRATLTVDRSTSTAYMELSSLRSEDTAVYFCKTSTFFFDYWGQGTTVTVSS |
| | (SEQ ID NO: 91) |
| L1-H5 | ELVMTQSPDSLAVSLGERATINCKSSQSLLNSGDQKNYLTWYQQKPGQPPKLLIYWAST |
| | RESGVPDRFSGSGSGTDFTLTISSLQAEDVAVYYCQNDYSYPLTFGQGTKLEIKGGGGSGGGGSG |
| | GGGSGGGGSQVQLVQSGAEVKKPGSSVKVSCKASGYTFTDHAIHWVRQAPGQGLEWIGHFSPGN |
| | TDIKYNDKFKGRATLTVDRSTSTAYMELSSLRSEDTAVYFCKTSTFFFDYWGQGTTVTVSS |
| | (SEQ ID NO: 92) |
| L2-H3 | DIVMTQSPDSLAVSLGERATINCKSSQSLLNSGDQKNYLTWYQQKPGQPPKLLIFWASTR |
| | ESGVPDRFSGSGSGTDFTLTISSLQAEDVAVYYCQNDYSYPLTFGQGTKLEIKGGGGSGGGGSGG |
| | GGSGGGGSQVQLVQSGAEVKKPGSSVKVSCKASGYTFTDHAIHWVRQAPGQGLEWMGHFSPGN |
| | TDIKYNDKFKGRVTITADKSTSTAYMELSSLRSEDTAVYYCKTSTFFFDYWGQGTTVTVSS |
| | (SEQ ID NO: 93) |
| L2-H5 | DIVMTQSPDSLAVSLGERATINCKSSQSLLNSGDQKNYLTWYQQKPGQPPKLLIFWASTR |
| | ESGVPDRFSGSGSGTDFTLTISSLQAEDVAVYYCQNDYSYPLTFGQGTKLEIKGGGGSGGGGSGG |
| | GGSGGGGSQVQLVQSGAEVKKPGSSVKVSCKASGYTFTDHAIHWVRQAPGQGLEWIGHFSPGNT |
| | DIKYNDKFKGRATLTVDRSTSTAYMELSSLRSEDTAVYFCKTSTFFFDYWGQGTTVTVSS |
| | (SEQ ID NO: 94) |
| L3-H3 | ELVMTQSPDSLAVSLGERATINCKSSQSLLNSGDQKNYLTWYQQKPGQPPKLLIFWASTR |
| | ESGVPDRFSGSGSGTDFTLTISSLQAEDVAVYYCQNDYSYPLTFGQGTKLEIKGGGGSGGGGSGG |
| | GGSGGGGSQVQLVQSGAEVKKPGSSVKVSCKASGYTFTDHAIHWVRQAPGQGLEWIGHFSPGNT |
| | DIKYNDKFKGRATLTVDRSTSTAYMELSSLRSEDTAVYFCKTSTFFFDYWGQGTTVTVSS |
| | (SEQ ID NO: 95) |

The amino acid sequences for the six selected in-house humanised scFv molecules (excluding residues corresponding to the Campath signal peptide and hexa-His tag).
(G₄S)₄ linker is in italic typeface.
Murine Kabat-defined CDRs are underlined.

### Example 2-Screening for Expression and Target Binding of Humanised Anti-TnMUC1 scFv Molecules (CAR Binder Moiety)

The objective of this study was to express twenty-six anti-TnMUC1 scFv-encoding constructs via transient transfection of 20 ml HEK 293 6E suspension culture and to characterise expression and target binding of secreted scFv present in the supernatants. Twenty-four of the constructs encoded in-house humanised scFv constructs. Additionally, two control constructs (10D1 and 10D2) were also examined. SDS-PAGE and Octet assays were used to assess relative expression levels of scFv proteins present in the supernatants. In addition, binding of the scFvs in supernatants to biotinylated un-glycosylated MUC1 and glycosylated TnMUC1 peptides was assessed.

### Methods

### HEK293 6E Suspension Culture

Suspension cultures of HEK 293 6E cells were in culture prior to transfection. A 250 ml stock culture was prepared in Freestyle 293 media supplemented with 10% Pluronic and 1% geneticin. The culture was split three times per week to a viable cell density of 5.00×10⁵ cell/ml.

### Octet Assay

A biotinylated 20 amino acid peptide containing Tn sugar residues at Serine and Threonine amino acids across the peptide (as described in Tarp, Sorensen et al, 2007) was designed and ordered from Cambridge Research Biosciences (CRB), termed “TnMUC1 peptide” (Biotin-PEG2-GV-T(AcNH-a-Gal)-S(AcNH-a-Gal)-APD-T(AcNH-a-Gal)-RPAPGS(AcNH-a-Gal)-T(AcNH-a-Gal)-APPAH-amide (SEQ ID NO: 76)). As a control, a non-glycosylated MUC1 peptide was also ordered with the same sequence, but with no Tn residues on the Serine and Threonine amino acids, termed “MUC1 peptide” (Biotin-[PEG2]-GVTSAPDTRPAPGSTAPPAH-amide (SEQ ID NO: 77)). The MUC1 and TnMUC1 peptides were resuspended to 5 mg/ml in 75% DMSO/25% PBS. The peptides were then aliquoted and stored at −80° C. The supernatants tested in the Octet assays are detailed in Table 8, with the exception of 6D10 and 6D11.

TABLE 8
References of anti-TnMUC1 scFv constructs
expressed in small-scale transfection

| Plasmid Lot ID | scFv encoded |
|---|---|
| 6D2 | L0-H1 |
| 6D3 | L0-H2 |
| 6D4 | L0-H3 |
| 6D5 | L0-H4 |
| 6D6 | L0-H5 |
| 6D7 | L1-H0 |
| 6D8 | L1-H1 |
| 6D9 | L1-H2 |
| 6D10 | L1-H3 |
| 6D11 | L1-H4 |
| 6D12 | L1-H5 |
| 6D13 | L2-H0 |
| 6D14 | L2-H1 |
| 6D15 | L2-H2 |
| 6D16 | L2-H3 |
| 6D17 | L2-H4 |
| 6D18 | L2-H5 |
| 6D19 | L3-H0 |
| 6D20 | L3-H1 |
| 6D21 | L3-H2 |
| 6D22 | L3-H3 |
| 6D23 | L3-H4 |
| 6D24 | L3-H5 |
| 6D25 | H3-L3 |
| 10D1 | VL-VH humanised control |
| 10D2 | VL-VH humanised control 2 |

Transient Transfection of scFv-Encoding Constructs into HEK 293 6E Suspension Culture

Transient transfections were performed in four separate batches. 20 μg of DNA of each plasmid construct was transfected using 293 Fectin transfection reagent into separate 20 ml cultures of HEK 293 6E cells at a viable cell density of 1.85×10⁶ cell/ml. A culture containing untransfected cells was prepared in one batch of transfections as a negative control; 293 Fectin transfection reagent was added to the culture but with no plasmid. The cultures were placed into a shaking 37° C. incubator at 124 rpm with 5% CO₂.

At 72 hours post-transfection, the viability (%) and viable cell density (cell/ml) of each culture were measured using a Vi-Cell cell counter and viability analyser (Beckman Coulter). As the cultures had reached 570% viability, the cultures were harvested via centrifugation at 2844×g for twenty minutes at 4° C. and filtered via 0.2 μm Millipore filter. The filtered supernatants were stored at 4° C. until required for subsequent analyses.

### Analysis of Supernatant by SDS PAGE

20 μl of each supernatant was mixed with 7 μl 4× reducing SDS PAGE sample buffer and samples heated at 98° C. for five minutes. 15 μl of sample was loaded per well on a NuPAGE Novex 4-12% Bis-Tris 12-well gel and the gel run in 1×MES SDS running buffer at 200V for approximately forty minutes. SeeBlue Plus2 protein marker was loaded on every gel as reference. Resolved protein bands were stained with InstantBlue protein stain and gels imaged.

### Analysis of Supernatant by Octet Assay

Quantification of Expression Levels of Humanised Anti-TnMUC1 scFv Proteins

Octet Ni-NTA sensors were soaked in 1×PBSF buffer for 10 minutes before use. 2×16 well heads were used and the amount of binding to Ni-NTA sensor captured at a single timepoint. Neat supernatants were loaded onto the Ni-NTA sensors and data taken at one report point for all constructs to obtain expression levels of the 6His tagged scFv proteins within the supernatant relative to each other.

130 μl of un-transfected media and each of the humanised anti-Tn MUC1 6×His-scFv supernatants were added to the assay plate, with 130 μl buffer in the blank wells.

This assay enabled the relative quantification of supernatants of humanised anti-Tn MUC1 scFv constructs, as the binding to Ni-NTA sensors in Octet Assay enables expression of different constructs to be measured only relative to each other.

Octet Binding Experiment—Humanised Anti-Tn MUC1 scFvs to Biotinylated MUC1 Peptides

Biotinylated MUC1 peptide (7-36-4) and TnMUC1 peptide (7-36-2) were diluted to 10 μg/ml in Octet Running Buffer (1×PBSF) and loaded onto Octet SA (streptavidin) sensors. The positive control proteins were tested in the Octet assay at a single concentration of 500 nM. All humanised anti-Tn MUC1 scFv expression supernatants were assayed neat.

Data was analysed using the data acquisition software fortéBIO version 8.0.2.5 and the data analysis software fortéBIO version 8.0.2.3.

### Results and Discussion

Transient Transfection of scFv-Encoding Constructs into HEK 293 6E Suspension Culture

Constructs encoding humanised anti-TnMUC1 scFvs were transiently transfected in to HEK 293 6E cells to generate culture supernatants containing secreted scFvs. In-house humanised anti-TnMUC1 scFvs showed varying levels of expression by SDS-PAGE. Further analysis of expression by Octet assay revealed that all molecules possessed notable expression above the levels seen for untransfected and blank samples. The viability (%) and viable cell density (cells/mL) for each culture was recorded at point of transfection (0 hours) and 72 hours post-transfection (Table 9).

TABLE 9
Transient Transfection Results
Summarized results of the small-scale transfections (0-36, 0-40, 0-43 and
0-45). Viability (%) and Viable cell density (cell/ml) were recorded at
point of transfection (0 H) and 72 hours (72 H). If the viability of the
culture was ≤70% the culture was harvested, and the supernatant was
collected.

| | | Hour Post-Transfection (H) | |
|---|---|---|---|
| Construct ID | Vi-Cell Reading | 0 H | 72 H |
| Untransfected | Viability (%) | 97.00% | 96.20% |
| Control | Viable Cell/ml | 1.85E+06 | 3.01E+06 |
| 6D2 | Viability (%) | 97.00% | 48.40% |
| | Viable Cell/ml | 1.85E+06 | 1.35E+06 |
| 6D3 | Viability (%) | 97.00% | 49.20% |
| | Viable Cell/ml | 1.85E+06 | 1.48E+06 |
| 6D4 | Viability (%) | 97.00% | 56.60% |
| | Viable Cell/ml | 1.85E+06 | 1.91E+06 |
| 6D5 | Viability (%) | 97.00% | 52.20% |
| | Viable Cell/ml | 1.85E+06 | 1.60E+06 |
| 6D6 | Viability (%) | 91.30% | 69.40% |
| | Viable Cell/ml | 1.85E+06 | 2.38E+06 |
| 6D7 | Viability (%) | 97.00% | 57.00% |
| | Viable Cell/ml | 1.85E+06 | 1.99E+06 |
| 6D8 | Viability (%) | 97.00% | 56.80% |
| | Viable Cell/ml | 1.85E+06 | 1.80E+06 |
| 6D9 | Viability (%) | 97.00% | 63.30% |
| | Viable Cell/ml | 1.85E+06 | 2.15E+06 |
| 6D10 | Viability (%) | 96.20% | 59.20% |
| | Viable Cell/ml | 1.85E+06 | 2.72E+06 |
| 6D11 | Viability (%) | 96.20% | 58.70% |
| | Viable Cell/ml | 1.85E+06 | 3.91E+06 |
| 6D12 | Viability (%) | 97.00% | 70.40% |
| | Viable Cell/ml | 1.85E+06 | 2.54E+06 |
| 6D13 | Viability (%) | 97.00% | 56.90% |
| | Viable Cell/ml | 1.85E+06 | 1.80E+06 |
| 6D14 | Viability (%) | 97.00% | 53.90% |
| | Viable Cell/ml | 1.85E+06 | 2.11E+06 |
| 6D15 | Viability (%) | 96.60% | 57.10% |
| | Viable Cell/ml | 1.85E+06 | 1.99E+06 |
| 6D16 | Viability (%) | 96.60% | 28.90% |
| | Viable Cell/ml | 1.85E+06 | 7.80E+05 |
| 6D17 | Viability (%) | 96.60% | 37.10% |
| | Viable Cell/ml | 1.85E+06 | 1.15E+06 |
| 6D18 | Viability (%) | 96.60% | 38.20% |
| | Viable Cell/ml | 1.85E+06 | 1.21E+06 |
| 6D19 | Viability (%) | 96.60% | 24.00% |
| | Viable Cell/ml | 1.85E+06 | 1.04E+06 |
| 6D20 | Viability (%) | 96.60% | 30.10% |
| | Viable Cell/ml | 1.85E+06 | 1.05E+06 |
| 6D21 | Viability (%) | 96.60% | 45.50% |
| | Viable Cell/ml | 1.85E+06 | 1.83E+06 |
| 6D22 | Viability (%) | 96.60% | 22.50% |
| | Viable Cell/ml | 1.85E+06 | 6.80E+05 |
| 6D23 | Viability (%) | 96.60% | 24.10% |
| | Viable Cell/ml | 1.85E+06 | 7.90E+05 |
| 6D24 | Viability (%) | 96.60% | 21.00% |
| | Viable Cell/ml | 1.85E+06 | 1.65E+06 |
| 6D25 | Viability (%) | 96.60% | 22.30% |
| | Viable Cell/ml | 1.85E+06 | 7.10E+05 |
| 10D1 | Viability (%) | 91.30% | 72.40% |
| | Viable Cell/ml | 1.85E+06 | 2.59E+06 |
| 10D2 | Viability (%) | 91.30% | 68.40% |
| | Viable Cell/ml | 1.85E+06 | 2.90E+06 |

### SDS-PAGE Analysis of Supernatants

Assessment of scFv expression levels in cell culture supernatant by SDS-PAGE and subsequent visual inspection of gels showed that levels varied significantly between molecules (data not shown).

A control supernatant generated from untransfected cells showed no scFv expression. All in-house humanised scFv molecules containing the light chain L0 (that contained no back-mutations; 6D2 through to 6D6) expressed robustly and comprised the major protein product in the corresponding supernatant. The presence of the heavy chain H0 (that contained no back-mutations; constructs 6D7, 6D13 and 6D19) or light chain L3 (constructs 6D19 through to 6D24) appeared to be correlated with reduced expression (as far as can be deduced by visual inspection). Similarly, the single molecule with a VH-VL orientation (H3-L3, construct 6D25) also appeared to have reduced apparent expression. Both control scFv molecules (encoded in constructs 10D1 and 10D2), showed robust levels of expression.

Octet Assays Analysis Including Binding of Humanised Anti-TnMUC1 scFvs to Biotinylated MUC1 Peptides

The qualitative expression levels assessed by visual inspection of SDS-PAGE gels and the relative quantitative expression levels determined by Octet assay appeared well correlated.

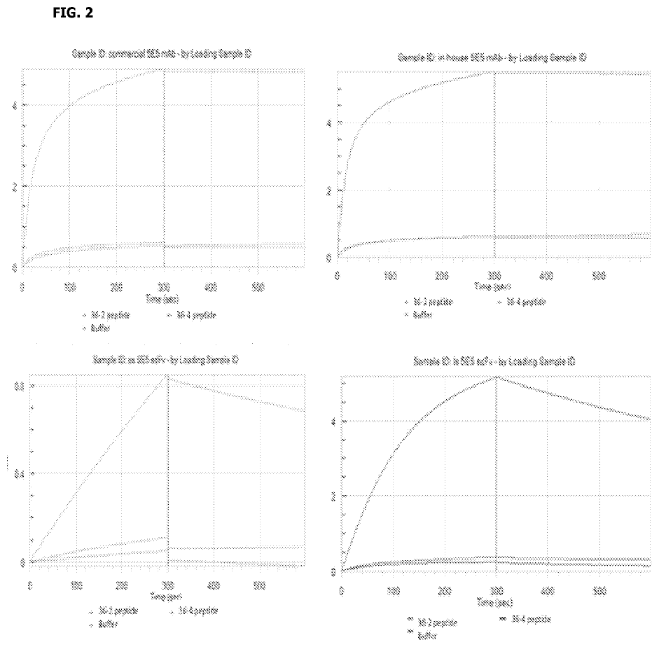

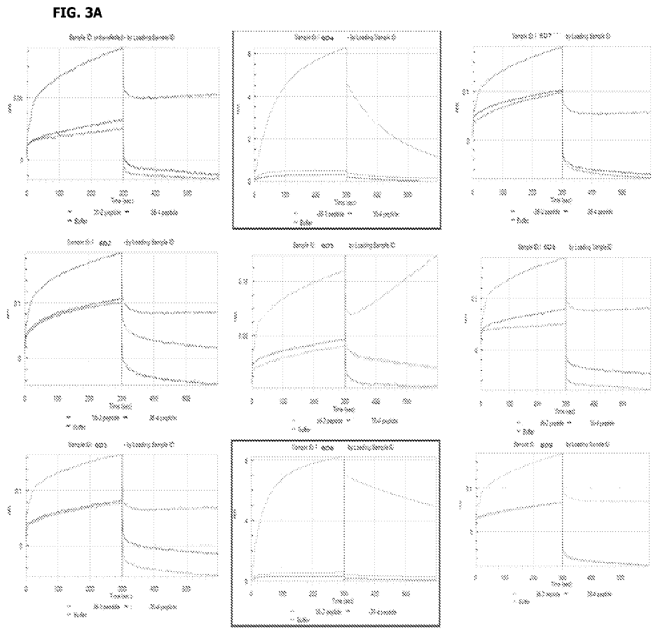

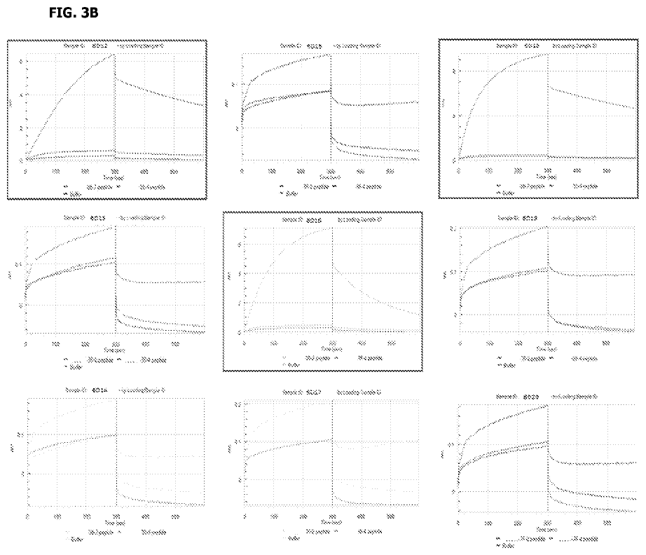

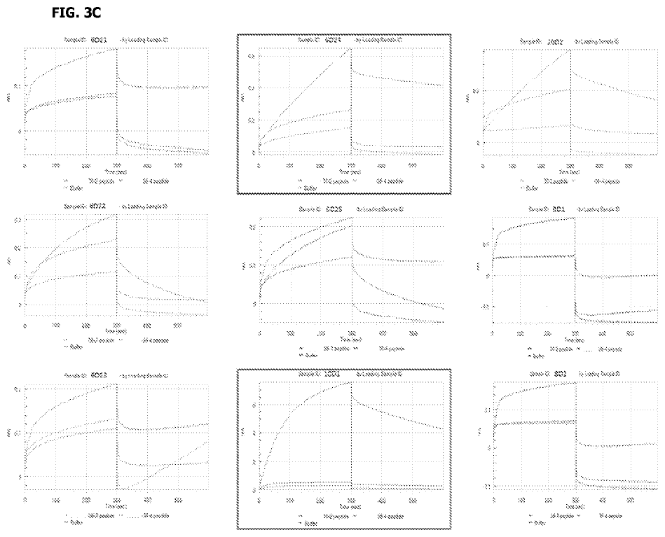

All positive controls bound specifically to the TnMUC1 peptide (36-2), with no binding to the unglycosylated MUC1 peptide (36-4) observed (FIG. 2). No binding to MUC1 peptide (36-4) was observed for any of the control proteins or supernatants tested. Selectivity for Tn-MUC1 peptide (36-2) binding was seen for a selection of the humanised anti-Tn-MUC1 scFv constructs (6D4, 6D6, 6D12, 6D16, 6D18 and 6D24), as well as control construct 10D1 (FIGS. 3A-3C).

Of the in-house humanised scFvs that showed binding to TnMUC1 peptide (36-2) by Octet binding assay all showed an appreciable Coomassie-stainable band by SDS-PAGE, except for supernatant arising from transfection of construct 6D24.

Both controls (10D1 and 10D2) showed robust expression by SDS-PAGE analysis and Octet assay. Strikingly, only the scFv expressed from 10D1 showed binding to TnMUC1 peptide (36-2). The two VL-VH scFvs encoded in these constructs are derived from two humanised whole antibodies (Ab1 and Ab2) that were shown to have comparable binding to TnMUC1 as murine mAb 5E5. An explanation for the lack of binding of the scFv encoded in 10D2 (scFv derived from Ab2) is that the reformatting of VL and VH domains in to a scFv has ablated the binding properties seen in the intact antibody.

Based on the results of the Octet assay examining specific binding of expressed humanised scFvs to TnMUC1 peptide (36-2), 6 in-house humanised scFvs (encoded in constructs 6D4, 6D6, 6D12, 6D16, 6D18 and 6D24), as well as the humanised control scFv (encoded in 10D1) were selected for large scale expression and purification.

### Example 3—Protein Purification Characterisation

Selected humanised scFvs were expressed in mammalian cells (HEK 293 6E cells) purified from the resulting supernatant via affinity chromatography.

### Experimental Preparation(s)

The scFv constructs used in this study are described in Tables 10 and 11. The scFv constructs used in this study contained a C-terminal 6×-His tag to allow for purification from cell supernatant via affinity chromatography. These constructs were triaged from twenty-six constructs previously expressed and tested (see Example 2 above). Suspension cultures of HEK 293 6E cells were in culture prior to transfection. A 250 ml stock culture was prepared in Freestyle 293 media supplemented with 0.1% Pluronic and 500 μg/ml geneticin. The culture was split three times per week to a viable cell density of 5.00×10⁵ cell/ml.

TABLE 10
Nomenclature and reference numbers for the shortlisted
anti-TnMUC1 single chain Fv Constructs (Plasmid and Protein)
Nomenclature and reference numbers for the shortlisted
anti-TnMUC1 single chain Fv Constructs (Plasmid and Protein)

| Plasmid ID | scFv Protein Purification Lot ID |
|---|---|
| 10D1 | 9P1 |
| 6D4 | 16P4 |
| 6D6 | 11P6 |
| 6D12 | 11P12 |
| 6D16 | 13P16 |
| 6D18 | 13P18 |
| 6D24 | 13P24 |
| | 28P24 |

TABLE 11
Humanised anti-TnMUC1 single chain Fv Construct Information
Humanised anti-TnMUC1 single chain Fv Construct Information including scFv
Protein ID number, amino acid sequence and molecular weight (Daltons)

| scFv Protein | | Length | Molecular |
|---|---|---|---|
| ID | | (Amino | Weight |
| (SEQ ID NO:) | Amino Acid Sequence | Acids) | (Daltons) |
| 88A | DIVMTQSPDSLAVSLGERATINCKSSQSLLNSG | 258 | 27708 |
| (SEQ ID NO: | DQKNYLTWYQQKPGQPPKLLIYWASTRESGVP | | |
| 78) | DRFSGSGSGTDFTLTISSLQAEDVAVYYCQNDY | | |
| | SYPLTFGQGTKVEIKGGGGSGGGGSGGGGSGG | | |
| | GGSQVQLVQSGAEVKKTGSSVKVSCKASGYTFT | | |
| | DHAIHWVRQAPGQALEWMGHFSPGNTDIKYN | | |
| | DKFKGRVTLTVDRSMSTAYMELSSLRSEDTAMY | | |
| | YCKTSTFFFDYWGQGTMVTVSSAAAHHHHHH | | |
| 82A | DIVMTQSPDSLAVSLGERATINCKSSQSLLNSG | 258 | 27555 |
| (SEQ ID NO: | DQKNYLTWYQQKPGQPPKLLIYWASTRESGVP | | |
| 79) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWMGHFSPGNTDIKYN | | |
| | DKFKGRVTITADKSTSTAYMELSSLRSEDTAVY | | |
| | YCKTSTFFFDYWGQGTTVTVSSAAAHHHHHH | | |
| 89A | DIVMTQSPDSLAVSLGERATINCKSSQSLLNSG | 258 | 27549 |
| (SEQ ID NO: | DQKNYLTWYQQKPGQPPKLLIYWASTRESGVP | | |
| 80) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWIGHFSPGNTDIKYND | | |
| | KFKGRATLTVDRSTSTAYMELSSLRSEDTAVYF | | |
| | CKTSTFFFDYWGQGTTVTVSSAAAHHHHHH | | |
| 94A | ELVMTQSPDSLAVSLGERATINCKSSQSLLNSG | 258 | 27563 |
| (SEQ ID NO: | DQKNYLTWYQQKPGQPPKLLIYWASTRESGVP | | |
| 81) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWIGHFSPGNTDIKYND | | |
| | KFKGRATLTVDRSTSTAYMELSSLRSEDTAVYF | | |
| | CKTSTFFFDYWGQGTTVTVSSAAAHHHHHH | | |
| 97A | DIVMTQSPDSLAVSLGERATINCKSSQSLLNSG | 258 | 27539 |
| (SEQ ID NO: | DQKNYLTWYQQKPGQPPKLLIFWASTRESGVP | | |
| 82) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWMGHFSPGNTDIKYN | | |
| | DKFKGRVTITADKSTSTAYMELSSLRSEDTAVY | | |
| | YCKTSTFFFDYWGQGTTVTVSSAAAHHHHHH | | |
| 19A | DIVMTQSPDSLAVSLGERATINCKSSQSLLNSG | 258 | 27533 |
| (SEQ ID NO: | DQKNYLTWYQQKPGQPPKLLIFWASTRESGVP | | |
| 83) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWIGHFSPGNTDIKYND | | |
| | KFKGRATLTVDRSTSTAYMELSSLRSEDTAVYF | | |
| | CKTSTFFFDYWGQGTTVTVSSAAAHHHHHH | | |
| 77A | ELVMTQSPDSLAVSLGERATINCKSSQSLLNSG | 258 | 27547 |
| (SEQ ID NO: | DQKNYLTWYQQKPGQPPKLLIFWASTRESGVP | | |
| 84) | DRFSGSGSGTDFTLTISSLQAEDVAVYYCQNDY | | |
| | SYPLTFGQGTKLEIKGGGGSGGGGSGGGGSGG | | |
| | GGSQVQLVQSGAEVKKPGSSVKVSCKASGYTFT | | |
| | DHAIHWVRQAPGQGLEWIGHFSPGNTDIKYND | | |
| | KFKGRATLTVDRSTSTAYMELSSLRSEDTAVYF | | |
| | CKTSTFFFDYWGQGTTVTVSSAAAHHHHHH | | |

### Experimental Protocol(s)

Transient Transfection of scFv-Encoding Construct into HEK 293 6E Suspension Culture

250 μg of DNA for each plasmid construct was transfected using 293 Fectin transfection reagent into separate 250 ml cultures of HEK 293 6E cells (at a viable cell density of 1.85×10⁶ cell/ml. The cultures were placed into a shaking 37° C. incubator at 124 rpm with 5% CO₂. At 48 and 72 hours the cultures were supplemented with 6.2 ml tryptone (200 g/I) and 6.2 ml 3M fructose respectively.

From 48 hours post-transfection the viability (%) and viable cell density (cell/ml) of each culture were measured every 24 hours using a Vi-Cell cell counter and viability analyser (Beckman Coulter). Once the cultures had reached 70% viability, the cultures were harvested via centrifugation at 4415×g for thirty minutes at 4° C. and filtered via 0.22 μm Millipore filter. The filtered supernatants were stored at 4° C. until required for protein purification.

Single-Step Affinity Protein Purification of scFv Construct and Subsequent Protein Characterisation

### Single-Step Affinity Protein Purification

The scFv proteins were purified from the resulting supernatants via ÄKTA Express system (ÄKTA). The supernatant was loaded at 5 ml/minute onto a 5 ml HisTrap Excel column pre-equilibrated with Buffer A (50 mM HEPES pH 7.5, 400 mM NaCl, 20 mM Imidazole). Once loaded, the column was washed in two column volumes of Buffer A at 5 ml/minute back to baseline. The proteins were eluted in a step elution of 50% Buffer B (50 mM HEPES pH 7.5, 400 mM NaCl, 1M Imidazole). The column was held in three column volumes of 50% Buffer B. During this step elution 0.5 ml fractions were collected in the purification of 88A, then 1 ml fractions were collected in all subsequent purifications. The elution step continued until returned to baseline followed by a washout step at three column volumes of 100% Buffer B.

A single peak was observed at 280 nM on the resulting chromatogram indicating the elution of the protein of interest. The fractions corresponding to this peak were pooled and transferred to a 5000 MW cut-off centrifugal concentrator. The sample was buffer exchanged from Buffer B into 60 ml PBS to separate the purified protein from the imidazole present in Buffer B. The samples were concentrated down to 1 ml. The concentration was determined via nanodrop and the purified protein was diluted in PBS to obtain a final concentration of 1 mg/ml. The final protein product was aliquoted and stored at −80° C. for future use. ScFv protein ID numbers were assigned to the purified proteins upon completion of characterisation (Table 11).

### SDS-PAGE

30 μl samples were taken from the fractions to be visualized via SDS-PAGE before they were pooled. 10 μl 4× reducing SDS-PAGE sample buffer was added and the samples were heated at 95° C. for ten minutes. 20 μl of each sample was loaded and run onto a NuPAGE Novex 4-12% Bis-Tris gel in 1×MES SDS running buffer at 200V for forty minutes. The gel was stained in Instant Blue on a shaking platform overnight, de-stained in water and imaged via Syngene gBox (Syngene).

The final purified protein was run on SDS-PAGE as a visual confirmation of protein size and purity.

Analytical Size Exclusion Chromatography (aSEC)

Analytical SEC was performed using two different instruments. The homogeneity of the purified proteins (97A, 19A and the first purification of 77A) was assessed using a Shimadzu LC-20AB liquid chromatography system with an SIL-20AC autosampler and a SPD-20A UV/Vis detector connected to a Superdex S75 10/300 column (Shimadzu). A 50 μl injection volume for each protein sample at a concentration of 1 mg/ml was run at 0.5 ml/minute in mqPBS running buffer. The LabSolutions software calculated the percentage of area of the detected peak(s). These values were used in Microsoft Excel to calculate their homogeneity.

The homogeneity of the purified proteins (88A, 82A, 89A, 94A and the second purification of 77A) was assessed using an Agilent 1260 infinity II connected to a TSKgel QC-PAK 300 column (Agilent). A 50 μl injection volume for each protein sample at a concentration of 1 mg/ml was run at 0.5 ml/minute in mqPBS running buffer. The Lab Advisor software was used to calculate the percentage of area and homogeneity of the detected peak(s).

### Mass Spectrometry

The intact mass of each protein was measured using the open-access mass spectrometer (QTOF-Ultima, Waters). A 0.5 μl injection volume for each protein sample at a concentration of 1 mg/ml was loaded onto the instrument. The resulting chromatograms were analysed via Masslynx software according to facility instructions.

### Peptide Mass Fingerprinting

The intact mass of 77A was not verified by mass spectrometry. Peptide Mass Fingerprinting (PMF) was performed to confirm the protein identity (Table 11).

The protein sample was prepared in reducing SDS PAGE sample buffer and 10 μg protein was loaded onto a NuPAGE Novex 4-12% Bis-Tris gel in 1×MES SDS running buffer at 200V for forty minutes. The gel was stained in Instant Blue on a shaking platform overnight, de-stained in water and imaged. The gel was submitted to an open-access facility for PMF.

### Results and Discussion

Transient Transfection of scFv Construct into HEK 293 6E Suspension Culture

The viability (%) and cell density of viable cells/ml for each transfected culture were recorded (Table 12). The results of these experiments were that seven humanised anti-TnMUC1 scFv-encoding constructs were successfully transfected into 250 ml cultures of HEK 293 6E suspension cells growing in the exponential phase. The scFv proteins were purified from the resulting supernatant and characterised.

TABLE 12
Transient Transfection Results
Summarised Results of Transient Transfection of seven single chain-Fv construct 250 ml HEK
293 6E cell culture. The following variables were measured at point of transfection 0 hours (0 H),
48 hours (48 H), 72 hours (72 H), 96 hours (96 H) 144 hours (144 H) and 168 hours (168 H):
Viability (%), Viable cell density (Cell/mL). Cells were harvested at ≤70%.

| Construct | Vi-Cell | Hour Post-Transfection (H) | | | | | |
|---|---|---|---|---|---|---|---|
| ID | Reading | 0 H | 48 H | 72 H | 96 H | 144 H | 168H |
| 10D1 | Viability (%) | 98.10% | 95.40% | 82.00% | | 8.90% | |
| | Viable Cells/mL | 1.82E+06 | 3.02E+06 | 2.51E+06 | | 2.60E+05 | |
| 6D4 | Viability (%) | 98.50% | 92.80% | 88.70% | 68.00% | | |
| | Viable Cells/mL | 2.13E+06 | 2.68E+06 | 2.98E+06 | 2.71E+06 | | |
| 6D6 | Viability (%) | 95.80% | 95.50% | 87.00% | 68.00% | | |
| | Viable Cells/mL | 1.85E+06 | 2.65E+06 | 3.07E+06 | 2.11E+06 | | |
| 6D12 | Viability (%) | 97.30% | 97.30% | 92.70% | 84.80% | | 40.70% |
| | Viable Cells/mL | 1.85E+06 | 3.04E+06 | 2.93E+06 | 2.81E+06 | | 1.84E+06 |
| 6D16 | Viability (%) | 98.30% | 97.50% | 96.00% | 88.90% | | 57.60% |
| | Viable Cells/mL | 1.85E+06 | 2.98E+06 | 3.33E+06 | 3.62E+06 | | 3.60E+06 |
| 6D18 | Viability (%) | 98.30% | 97.90% | 95.60% | 90.80% | | 61.80% |
| | Viable Cells/mL | 1.85E+06 | 3.22E+06 | 3.39E+06 | 3.90E+06 | | 4.65E+06 |
| 6D24 | Viability (%) | 99.00% | 97.70% | 94.80% | 61.80% | | |
| | Viable Cells/mL | 1.85E+06 | 2.87E+06 | 3.10E+06 | 2.06E+06 | | |
| 6D24 | Viability (%) | 98.20% | 98.00% | 94.80% | 87.60% | | 71.60% |
| | Viable Cells/mL | 1.85E+06 | 3.18E+06 | 3.74E+06 | 4.45E+06 | | 5.22E+06 |

Single-Step Affinity Protein Purification of scFv Construct and Subsequent Protein Characterisation

Six humanised anti-TnMUC1 scFv molecules plus one control were successfully expressed via mammalian expression in HEK 293 6E cells. These proteins were successfully purified from the resulting supernatant (250 mL supernatant) via affinity chromatography. 5 μg aliquots of the purified samples in the batch were run under reducing conditions via SDS PAGE, displaying the purity and correct molecular weight of the purified proteins (FIG. 4). These attributes were supported by aSEC and mass spectrometry analyses (data not shown). The purified scFv proteins were used in further in vitro experiments, the results of which were used to select scFv-encoding regions for reformatting into CARs.

### Example 4—Biacore Analysis of Humanised Anti-TnMUC1 scFv Proteins

Binding of purified humanised anti-TnMUC1 scFv proteins to TnMUC1 and sTnMUC1 peptides was tested in Biacore assays. The epitope within the TnMUC1 peptide to which the scFvs bind was also determined.

### Methods

### Experimental Preparation(s)

Generation of scFv Proteins, Preparation of Peptides

The humanised scFv proteins were generated as detailed above in Example 3 (see Table 13 for details on humanised scFv molecules tested).

TABLE 13
Humanised scFv proteins tested in BIAcore assays

| Construct | Construct | | CAR name* (zsGreen |
|---|---|---|---|
| DNA Name | Protein Name | Protein ID | marker) |
| 10D1 | 9P1 | 88A | MB020 |
| 6D4 | 16P4 | 82A | MB021 |
| 6D6 | 11P6 | 89A | MB023 |
| 6D12 | 11P12 | 94A | MB022 |
| 6D16 | 13P16 | 97A | MB024 |
| 6D18 | 13P18 | 19A | MB025 |
| 6D24 | 13P24 | 77A | MB026 |

*the equivalent CAR containing this scFv protein sequence which is co-expressed with zsGreen marker. CARs MB020, MB021, MB022, MB023, MB024, MB025, MB026 are co-expressed with a zsGreen marker. The ZsGreen marker is expressed from the same vector as the CAR, but is expressed separately from the CAR via cleavage by a P2A peptide sequence.

Biotinylated 20 amino acid peptides containing Tn sugar residues at Serine and Threonine amino acids across the peptide (as described in Tarp, Sorensen et al, 2007) were designed and ordered from Cambridge Research Biosciences (CRB). As a control, a non-glycosylated MUC1 peptide was also ordered with the same sequence, but with no Tn or STn residues on the Serine and Threonine amino acids. In order to determine the epitope of where the humanised anti-TnMUC1 scFv proteins bind the Tn peptide, a further two differentially glycosylated peptides were ordered, with one containing Tn sugar residues on Ser/Thr amino acids of the peptide reported in the literature to be where the scFv proteins bind, and one containing Tn sugar residues on Serine and Threonine amino acids where the scFv proteins reportedly do not bind (as described in Tarp, Sorensen et al, 2007).

The sequences and ID numbers of these peptides are detailed in Table 14 and represented schematically in FIG. 5.

TABLE 14
MUC1, TnMUC1 and STnMUC1 peptide information

| | | | | Stock |
|---|---|---|---|---|
| Name | ID | Sequence | MW | Concentration |
| MUC1 | 71A | Biotin-[PEG2]- | 2371.505 | 5 mg/ml; |
| | | GVTSAPDTRPAPGSTAPPAH- | | 2250 μM |
| | | amide (SEQ ID NO: 77) | | |
| TnMUC1 | 80B | Biotin-[PEG2]-GV-T(AcNH-a-Gal)- | 3271.493 | 5 mg/ml; |
| peptide | | S(AcNH-a-Gal)-APD-T(AcNH-a- | | 1500 μM |
| (fully | | Gal)-RPAPG-S(AcNH-a-Gal)- | | |
| glycosylat | | T(AcNH-a-Gal)-APPAH-amide | | |
| ed) | | (SEQ ID NO: 76) | | |
| TnMUC1 | 32A | Biotin-PEG2-GV-TSAPD- | 2662.3 | 5 mg/ml; |
| peptide 1 | | TRPAPGS(AcNH-a-Gal)-T(AcNH- | | 1878 μM |
| | | a-Gal)-APPAH-amide (SEQ ID | | |
| | | NO: 85) | | |
| TnMUC1 | 21A | Biotin-PEG2-GV-T(AcNH-a-Gal)- | 2865.3 | 5 mg/ml; |
| peptide 2 | | S(AcNH-a-Gal)-APD-T(AcNH-a- | | 1745 μM |
| | | Gal)-RPAPGSTAPPAH-amide | | |
| | | (SEQ ID NO: 86) | | |
| STnMUC1 | 06A | Biotin-[PEG]2-GVTSAPDTRPAPG- | 3360.399 | 0.8 mg/ml; |
| | | [Ser(Sial-AcNH-a-Gal)]-[Thr(Sial- | | 238 μM |
| | | AcNH-a-Gal)]-APPAH-amide (SEQ | | |
| | | ID NO: 87) | | |

### Capture Level Determination—Preparing the Surface of the CAPture Chip

In the assay format used in these experiments, the biotinylated peptides were captured onto the CAP chip (ligand) to generate an active surface and the scFv proteins were passed over the chip surface (analyte). The active surface for kinetic measurements is designed to ensure low maximum analyte binding capacity (Rmax). This facilitates measurement of fast on and off rates by reducing analyte transport limitations. Suitable values for Rmax are in the range 20-100 RU (Response Units) for protein-protein interactions in the highest performance Biacore systems.

The theoretical analyte binding capacity of the surface in RU is given by the following equation:

Target capture level=Rmax×Mwt ligand/stoichiometry×Mwt analyte

In the case of these experiments:
Ligand MW (peptide)=2500 Da
Analyte MW (anti-Tn MUC1 scFv)=27000 Da*
Stoichiometry: 1
Rmax=100 (to prevent re-binding events)

Target capture level=Rmax×Mwt ligand/stoichiometry×Mwt analyte

=100×2500/1×27000

=11RU

*this molecular weight was used for all scFv proteins tested as an average molecular weight for the scFv molecules.

By the above calculation, a theoretical 11RU of each peptide would need to be captured for each cycle.

### Experimental Protocol(s)

### Capture Level Determination of TnMUC1 and STnMUC1 Peptides

The CAP chip (from the Biotin CAPture kit) was docked into BIAcore T200 instrument to hydrate overnight, as per manufacturer instructions. When the chip is docked (for the first time or after storage), the surface of the sensor chip needs to be conditioned with 3×1 minute injections of Regeneration Solution (from the Biotin CAPture kit). Regeneration Solution was prepared by mixing 3 parts of Regeneration Stock 1 (8M Guanidine-HCl) with 1-part Regeneration Stock 2 (1M NaOH). Both the reference and active flow cells of the sensor chip were conditioned with 3×60 second injections of Regeneration Solution at a flow rate of 10 μl/min.

All biotinylated peptides were diluted in BIAcore Running Buffer (1×HBS-EP⁺=10 mM Hepes pH7.6, 150 mM NaCl, 3 mM EDTA, 0.05% polysorbate 20) for initial capture onto the CAPture chip to ascertain binding levels required, as detailed in the equation described above. The capture level determination experiment was carried out on the active flow cell only. As the reference flow cell in this assay format contains the Biotin CAPture reagent, it was not possible to check for any non-specific binding to the reference flow cell, as the biotinylated peptide would bind both active and reference flow cells.

Biotin CAPture Reagent (from the Biotin CAPture kit) was captured onto the active flow cell at a flow rate of 2 μl/min with a contact time of 300 seconds followed by a 60 second stabilisation period.

The ligand (biotinylated peptide) was passed over the active flow cell with an association time of 120 seconds at a flow rate of 10 μl/min, followed by a dissociation phase of 60 seconds where BIAcore Running Buffer was injected at a flow rate of 10 μl/min.

The Biotin CAPture chip was regenerated using Regeneration Solution, which was prepared as described previously.

The Y-values obtained for the capture of each of the peptides from the Capture experiments were used to calculate the length of capture required to obtain a capture level close to 11RU. Initially, TnMUC1 (36-2) and MUC1 (36-4) peptides were diluted and captured onto the Biotin CAPture reagent on the CAP chip. The Y-value obtained in this experiment was used to determine how long the peptides should be captured for in the Binding Analysis assays to provide a capture level close to the theoretical value required, as detailed above. As the amount captured in practice can be lower than the theoretical values (due to not all protein being active), a higher capture level was aimed for in these experiments, in order to ensure a capture level close to 11RU was obtained.

### Binding Assays

Biotin CAPture Reagent was captured onto the active and reference flow cells for 300 seconds at 2 μl/min. The biotinylated peptides were diluted in BIAcore Running Buffer (as described above) and captured onto the active flow cell only, at a flow rate of 10 μl/min to obtain the capture level calculated in the equation detailed above.

The humanised anti-TnMUC1 scFv proteins were passed over the reference and active flow cells at 500, 125, 31.25, 7.8, 1.9, 0 nM for binding to TnMUC1 and MUC1 peptides with a 300 second association followed by a 900 seconds dissociation with BIAcore Running Buffer at a flow rate of 30 μl/min.

For binding to the STnMUC1 peptide, all of the humanised anti-TnMUC1 scFv proteins were passed over the reference and active flow cells at 1000, 250, 62.5, 15.6, 3.9, 0.98, 0 nM for binding to STnMUC1 and MUC1 peptides with a 520 seconds association followed by a 900 seconds dissociation with 1×HBS-EP⁺ at a flow rate of 30 μl/min. Due to the expected lower affinity of the humanised anti-TnMUC1 scFv proteins for the STnMUC1 peptide compared to the TnMUC1 peptide, a higher top concentration of scFvs was used for these experiments. In order to obtain better curvature for the sensorgrams, the association time for these experiments was also increased from 300 seconds to 520 seconds. The dissociation time of 900 seconds was sufficient for these experiments.

In both cases, the Biotin CAPture chip was regenerated as described above.

N=2 data was generated for each of the humanised scFv proteins for binding to all TnMUC1 and STnMUC1 peptides. The MUC1 peptide was used as a negative control in all experiments.

BIAcore T200 Evaluation Software v3.0 was used to analyse the data. The data obtained has been fitted to a 1:1 binding model with the Rmax parameter set to local. As this is a capture experiment, a local Rmax parameter would be truly reflective as the protein is recaptured every time and theoretically surface is slightly different at each recapture, possibly producing slightly different capture each time. Kinetic Parameters (K_{D}, k_{d} and kₐ) were calculated for all the humanised anti-TnMUC1 scFv proteins.

### Results and Discussion

Binding of Humanised Anti-TnMUC1 scFv Proteins to TnMUC1 (36-2/65-1) and MUC1 (36-4) Peptides

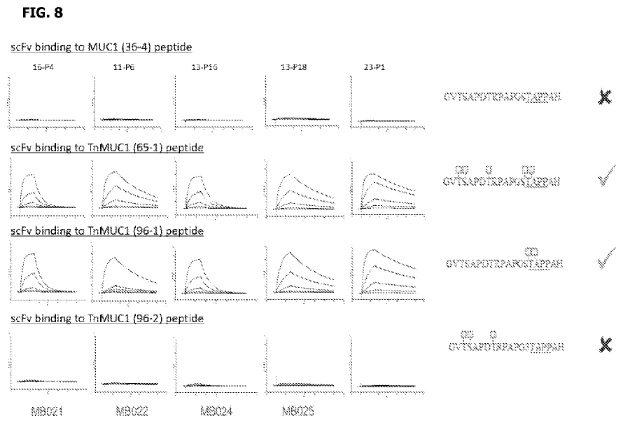

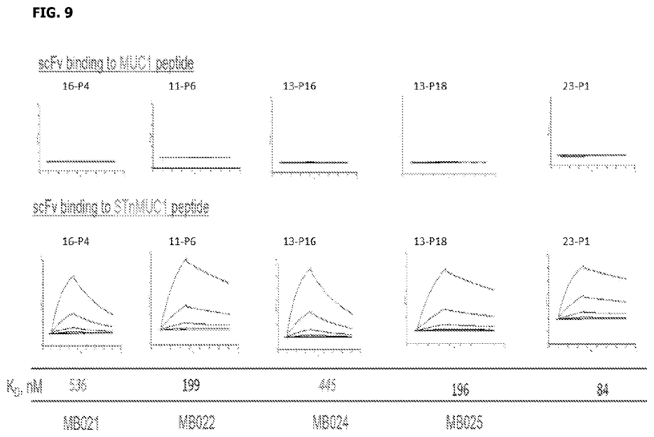

All seven purified humanised anti-TnMUC1 scFv proteins were tested for binding to TnMUC1 (36-2) and MUC1 (36-4) peptides. Specific binding of the humanised anti-TnMUC1 scFv proteins to the fully glycosylated TnMUC1 peptide and STnMUC1 peptide, with no binding seen to MUC1 peptides (FIG. 7, FIG. 9 and Table 15). Reproducible n=2 data was obtained for all experiments, and the values obtained have been averaged. Table 15 shows the ka, kd and KD values obtained for the binding of all humanised anti-TnMUC1 scFv proteins to the TnMUC1 peptide (36-2). Comparable ka, kd and KD values were obtained for all of the scFvs with the exception of scFv proteins 16-P4 and 13-P16, which from the sensorgram shape as can be seen in FIG. 7 and the values detailed in Table 15, have a faster k_{d} (off rate) and in turn a lower K_{D} for the fully glycosylated TnMUC1 peptide (36-2), compared to the other scFv proteins.

TABLE 15
Binding of humanised anti-TnMUC1 scFv proteins to TnMUC1 (36-2)

| | | | | ka (1/Ms) | kd (1/s) | KD (M) |
|---|---|---|---|---|---|---|
| Sample | ka (1/Ms) | kd (1/s) | KD (M) | Average | Average | Average |
| 9P1 | 6.88E+04 | 1.57E−03 | 2.28E−08 | 6.78E+04 | 1.56E−03 | 2.30E−08 |
| | 6.88E+04 | 1.57E−03 | 2.28E−08 | | | (23 nM) |
| | 6.59E+04 | 1.53E−03 | 2.33E−08 | | | |
| 16P4 | 3.56E+04 | 6.94E−03 | 1.95E−07 | 3.51E+04 | 6.87 E−03 | 1.96E−07 |
| | 3.45E+04 | 6.80E−03 | 1.97E−07 | | | (196 nM) |
| 11P6 | 5.38E+04 | 1.38E−03 | 2.57E−08 | 5.15E+04 | 1.37E−03 | 2.67E−08 |
| | 4.91E+04 | 1.35E−03 | 2.76E−08 | | | (26.7 nM) |
| 11P12 | 2.94E+04 | 1.58E−03 | 5.36E−08 | 2.84E+04 | 1.55E−03 | 5.44E−08 |
| | 2.73E+04 | 1.51E−03 | 5.52E−08 | | | (54 nM) |
| 13P16 | 3.95E+04 | 6.54E−03 | 1.66E−07 | 3.78E+04 | 6.53E−03 | 1.74E−07 |
| | 3.61E+04 | 6.52E−03 | 1.81E−07 | | | (174 nM) |
| 13P18 | 1.94E+04 | 1.31E−03 | 6.76E−08 | 1.84E+04 | 1.30E−03 | 7.09E−08 |
| | 1.74E+04 | 1.29E−03 | 7.42E−08 | | | (70.9 nM) |
| 13P24 | 2.87E+04 | 1.33E−03 | 4.64E−08 | 2.87E+04 | 1.33E−03 | 4.64E−08 |
| | 2.87E+04 | 1.33E−03 | 4.64E−08 | | | (46.4 nM) |

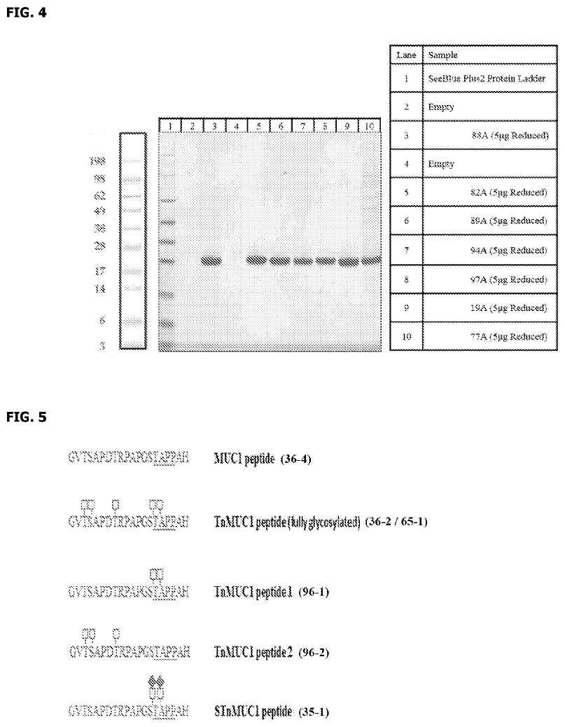

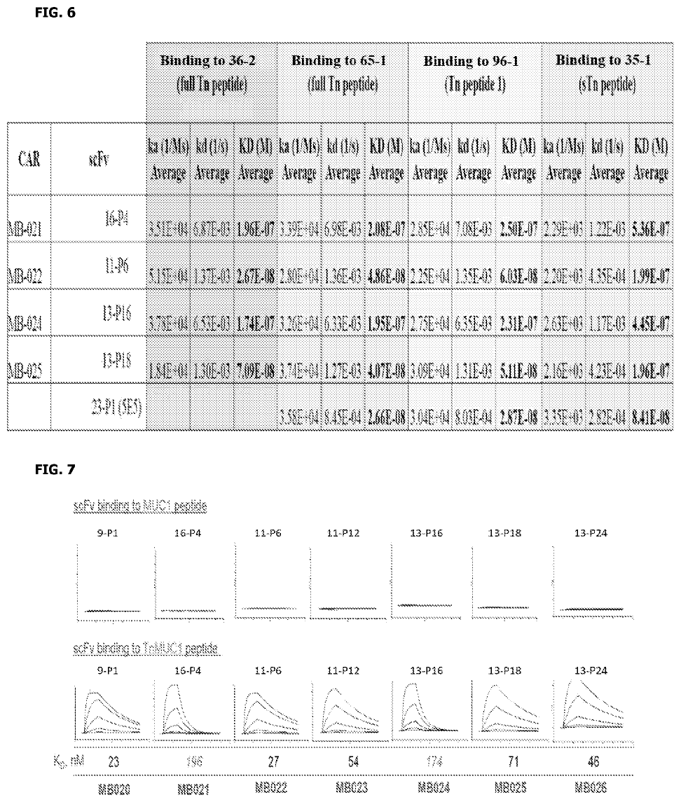

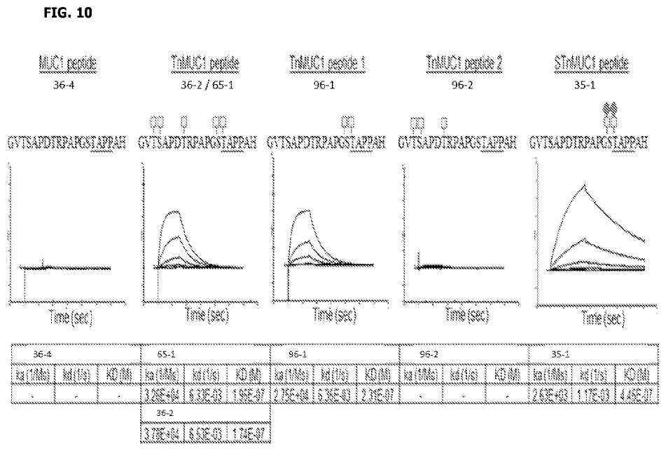

Determination of the Epitope for the Humanised Anti-TnMUC1 scFv Proteins within the TnMUC1 Peptide Four scFv proteins (16-P4, 11-P12, 13-P16, 13-P18), as well as the murine 5E5 scFv protein (23-P1) obtained from Creative Biolabs, were all tested for binding to the MUC1 (36-4) and the fully glycosylated TnMUC1 peptide (65-1), as well as the differentially glycosylated TnMUC1 peptides (96-1 and 96-2), to determine the binding epitope of the humanised scFv proteins on the TnMUC1 peptide. These experiments showed equivalent binding of the humanised anti-TnMUC1 scFv proteins to the fully glycosylated TnMUC1 peptide (65-1) and the differentially glycosylated TnMUC1 peptide 1 (96-1), with no binding observed to the MUC1 peptide (36-4) or to TnMUC1 peptide 2 (96-2). The data obtained in these experiments has confirmed the epitope of the humanised anti-TnMUC1 scFv proteins within the peptides tested. The sensorgrams shown in FIG. 8 show comparable association and dissociation of the humanised anti-TnMUC1 scFv proteins to the two peptides (65-1 and 96-1), with the values for the kinetic parameters shown in FIG. 6. The murine 5E5 scFv protein also showed comparable binding to the fully glycosylated peptide (65-1) and the differentially glycosylated peptide (96-1), as can be seen in FIG. 10. Two bathes of fully glycosylated TnMUC1 peptides were tested (65-1 and 36-2) with comparable binding of the humanised anti-TnMUC1 scFvs seen between the batches. This data is summarised in FIG. 6.
Binding of Humanised Anti-TnMUC1 scFv Proteins to STnMUC1 and MUC1 Peptides

As described above, four scFv proteins as well as the murine 5E5 scFv protein were all tested for binding to the MUC1 and the STnMUC1 (35-1) peptides. The humanised scFv proteins were shown to bind specifically to the STn peptide (35-1), with no binding seen to the MUC1 peptide (36-4). They were however, found to have an approximately 10 fold lower affinity for the STnMUC1 peptide compared to the murine 5E5 scFv protein generated at Creative Biolabs, as can be seen in FIGS. 6 and 9. As seen in the TnMUC1 binding experiments, scFv proteins 16P4 and 13P16 were shown to have a faster kd (off rate) and in turn a lower KD for the STn peptide (35-1) compared to scFv proteins 11P12 and 13P18, as can be seen in FIGS. 6 and 9.

In sum, specific binding of the humanised anti-TnMUC1 scFv proteins to the TnMUC1 (36-2, 65-1, 96-1) and STnMUC1 (35-1) peptides was observed, with no binding to the MUC1 (36-4) peptide observed. This assay has highlighted two distinct binding profiles obtained for the humanised scFv proteins, with 16P4 and 13P16 being shown to have a faster kd (off rate) and 11P12 and 13P18 as having a comparatively slower kd (off rate). All humanised scFv proteins tested and the murine 5E5 scFv protein were shown in these experiments to bind the same epitope within the TnMUC1 peptide.

Only the scFv fragment of the CAR molecules were tested in this experiment, the final CAR molecules cannot be tested in this assay system. Further characterisation of the equivalent CARs binding to these peptides was carried out in the peptide stimulation assay described in Example 12. The binding for the 13P16 scFv to all of the peptides tested is summarised in FIG. 10.

### Example 5—Cell Binding Methods

Binding of purified scFv proteins to TnMUC1 positive and negative tumour cell lines was determined by flow cytometry. The aim of this study was to differentiate the humanised scFv regions of the CAR constructs based on the relative binding efficacies of titrated scFv proteins in TnMUC1 positive and negative tumour cell lines as determined by flow cytometry.

### Experimental Preparation(s)

### Adherent Cell Culture

Adherent MDA-MB-468 and PC3 cell lines were maintained in culture by passaging twice weekly at a split ratio of 1:3. Cell monolayers were washed with DPBS before incubating for a few minutes with TrypLE Express (TE). Once detached, cells were re-suspended in appropriate cell culture media (final volume equal to 5× volume of TE used) and seeded into fresh cell culture flasks. Cells were kept in humidified incubator at 37° C. with 5% CO₂ until required.

### Suspension Cell Culture

Jurkat cells were maintained in culture by passaging twice weekly by transferring 10% of the confluent cell culture into a fresh cell culture flask and then adding an appropriate volume of cell growth media (to give a final split ratio of 1:10). Cells were kept in a humidified incubator at 37° C. with 5% CO₂ until required.

### Cell Preparation for Flow Cytometry Assay

Cells were detached and cells were re-suspended in appropriate cell culture media or transferred to Falcon tube. A sample of each cell suspension was then diluted 1:5 with DPBS (to give a final volume of 0.5 mL) and the number of viable cells per mL (based on trypan blue exclusion) were determined for each cell suspension using the Vi-CELL XR Cell Viability Analyser (Default cell type setting used). Cell suspensions were then diluted in appropriate cell culture media and seeded into fresh cell culture flasks at a density of 6.25×10⁴ viable cells per cm². Cells were incubated in a humidified incubator at 37° C. with 5% CO₂ for 48 hours (or until required). On the day of assay, cells were detached from flasks, resuspended in appropriate cell culture media (if appropriate) and the number of viable cells per mL counted as described previously. Cell suspensions were then centrifuged at 500×g for 5 minutes before resuspending cell pellets in FACS Buffer at a density of 4×10⁶ viable cells per mL.

### Experimental Protocol(s)

### Cell Binding Assay

scFv proteins were diluted to 62.5 nM in DPBS and dispensed into row A of a 96-well V-bottom plate before performing a 1:4 serial dilution in FACS Buffer down the plate to give 25 μL (final volume) per well (FACS Buffer in row H only). Anti-Hist-PE detection antibody diluted 1:5 in DPBS and 25 μL per well dispensed into wells containing serially-diluted scFv proteins before incubating plate for 1 hour at 4° C. protected from light to pre-complex scFv proteins with the anti-Hist-PE detection antibody. Cell suspensions prepared as described previously then dispensed into plate (50 μL per well) before incubating the plate for a further hour at 4° C. protected from light. Cells washed by resuspending cells in 200 μL per well (maximum volume) FACS Buffer and centrifuging cells at 500× g for 5 minutes before removing supernatant and repeating wash step twice more to wash cells. Cell pellets resuspended in 200 μL per well FACS Buffer with 1 μg per mL (final) DAPI and 2 mM (final) EDTA before acquiring data using CytoFLEX S flow cytometer.

### Target Expression Assay

Cell suspensions (prepared as described previously) dispensed into a 96-well plate V-bottom plate (25 μL per well) and volume made up to 200 μL per well with FACS buffer before centrifuging cells at 500× g for 5 minutes. Cells were resuspended in 100 μL per well primary antibody diluted to 5 μg per mL in FACS Buffer (or equal concentration of isotype antibody/FACS Buffer only as appropriate). Cells were incubated for 45 minutes at 4° C. Cells were then washed 3× with FACS Buffer as described previously before resuspending cell pellets in 100 μL per mL goat anti-mouse PE-conjugated secondary antibody diluted 1:1000 in FACS buffer and incubating cells for a further 45 minutes at 4° C. Cells were then washed, resuspended in FACS Buffer supplemented with DAPI and EDTA and data acquired as described previously.

The humanized scFv proteins tested were: 9P1 (88A), 16P4 (82A), 11P6 (89A), 11P12 (94A), 13P16 (97A), 13P18 (19A), and 13P24 (7A) (see Table 13 above).

### Data Analysis

### Cell Binding Assay Data

Raw data was analysed using FlowJo. Well samples grouped by cell line and gates set to discriminate live single positive cells using cells only well sample for each cell line before applying gates to well samples in same group. Median Fluorescent Intensity (MFI) values were exported into Prism and transformed into log scale (i.e. X=Log(X)) before fitting data using a variable slope 4 parameter equation: Y=Bottom+(Top−Bottom)/(1+10\hat{ }((−pEC50−X)*HillSlope)). MFI values were removed prior to curve fitting where evidence of hook effect at top concentration of humanised scFv proteins was observed in Jurkat cells. Standard error values calculated by Prism. The data generated for this report is not instrumental in the selection of a lead molecule, therefore no statistical analysis methods were performed on this data.

### Target Expression Assay Data

Raw data was analysed using FlowJo as described previously. Percent PE-positive values in isotype control wells were subtracted from primary antibody wells for each cell line and plotted using Prism.

### Results and Discussion

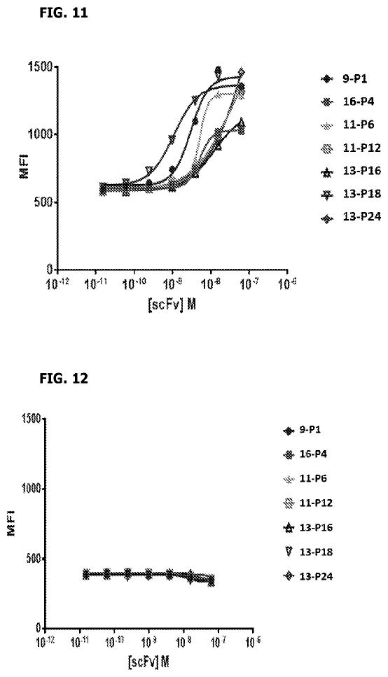

The data demonstrates that all of the humanised scFv proteins selectively bind to TnMUC1-expressing tumour cells (TnMUC1-positive MDA-MB-468 tumour cells) in a scFv concentration and TnMUC1 expression dependent manner. See representative data in FIG. 11. With the exception of 11P6 (89A) and 11P12 (94A) mean EC50 and pEC50 values were calculated for all humanised scFv proteins based on n=2 experiments, with mean EC50 concentrations ranging from 1.68 nM for 13P18 (19A) to 84.38 nM for 28P24 (77A); EC50 concentrations for 11P6 (89A) and 11P12 (94A) were 4.968 and 34.2 nM respectively (based on n=1 experiment data). See Table 16.

The 13P16 (97A) humanised scFv protein showed reduced binding potency compared to 9P1 (88A), 16P4 (82A), 11P6 (89A) and 13P18 (19A) with EC50 values around 11 nM versus EC50 values of 5 nM or less for the most potent humanised scFv proteins.

TABLE 16
pEC50 and EC50 values for scFv protein binding in
MDA-MB-468 cells

| | pEC50 | | Mean | [EC50] M | | Mean |
|---|---|---|---|---|---|---|
| scFv | 75-13 | 75-14 | pEC50 | 75-13 | 75-14 | [EC50] M |
| 9P1 | 8.52 | 8.50 | 8.51 | 2.996E−09 | 3.184E−09 | 3.090E−09 |
| 16P4 | 8.29 | 8.65 | 8.47 | 5.088E−09 | 2.239E−09 | 3.664E−09 |
| 11P6 | 8.30 | ~8.396 | 8.30 | 4.968E−09 | ~4.022e−9 | 4.968E−09 |
| 11P12 | 7.47 | ~2.740 | 7.47 | 3.420E−08 | ~0.001821 | 3.420E−08 |
| 13P16 | 7.92 | 7.95 | 7.94 | 1.199E−08 | 1.119E−08 | 1.159E−08 |
| 13P18 | 8.97 | 8.64 | 8.80 | 1.076E−09 | 2.284E−09 | 1.680E−09 |
| 13P24 | 6.83 | 7.68 | 7.25 | 1.476E−07 | 2.115E−08 | 8.438E−08 |

EC50 values were generated by Prism for the binding of 9P1 (88A), 11P6 (89A), 11P12 (94A), 13P18 (19A) humanised scFv proteins in TnMUC1-negative control PC3 cells (data not shown). Representative data in FIG. 12 also shows some evidence of a hook effect at the top scFv protein concentrations; however, the data in FIG. 12 along accompanied negative Hill slope values (Hill slope values not shown) suggest that there is no evidence of humanised scFv protein binding in PC3 cells.

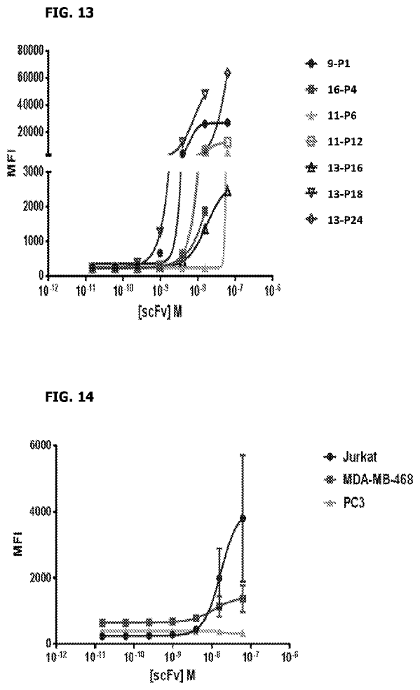

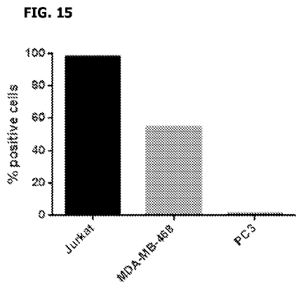

Representative fitted MFI data in FIG. 13 shows increased binding for all the humanised scFv proteins in highly-positive TnMUC1-positive control Jurkat cells compared to MDA-MB-468 cells (FIG. 11) corresponding with representative TnMUC1 membrane expression data for Jurkat, MDA-MB-468 and PC3 cells (FIG. 15).

Fitted curves from n=2 experiments in FIG. 14 specifically demonstrate selective binding of 13P16 (97A) to TnMUC-positive MDA-MB-468 and highly TnMUC-positive Jurkat cells; again, correlating with representative TnMUC1 membrane expression data for Jurkat, MDA-MB-468 and PC3 cells (FIG. 15). Evidence of a hook effect was observed at the top concentration of 16P4 (82A) and 13P18 (19A) in duplicate experiments in Jurkat cells, and also for 9P1 (88A) and 11P6 (89A) in 1 of 2 repeat experiments only (MFI values removed prior to curve fitting).

### Example 6—Preclinical Safety

The objective of these studies was to assess off-target binding of TnMUC1 CAR-T cells using a plasma membrane protein array.

### Methods

### Experimental Protocols

### Generating CAR-T Cells to Support Pilot Study

Peripheral blood monocytes (PBMCs) were isolated from human blood using Histopaque (Sigma, catalogue number 10771) and Accuspin tubes (Sigma, catalogue number A7054) in accordance with the manufacturer's instructions. Cells were resuspended in TEXMacs media. IL-2 (100 IU/ml) and TransAct T cell activation beads (1:100 dilution) were added to the PBMCs and cells incubated in a humified incubator at 37° C. with 5% CO₂ for two days. PBMC were transduced with BCMA vector, BCMA-030, with a MOI of 2.75 and incubated at 37° C. with 5% CO₂ for two days. Cells were maintained in TEXMacs media and IL-2 at 100 IU/ml throughout the culture period. Cells were harvested 13 days after transduction and frozen at 1×10⁸ cells/ml. Untransduced cells were generated as a negative control. T cell batches were generated from three donors.

Transduction efficiency was determined by detecting CAR expression with Alexa Fluor 647 conjugated BCMA-Fc using flow cytometry (MACSQuant Analyser 10) and the APC channel. Data was analysed using FlowJo v10.1.

### Generating CAR-T Cells to Support Pre-, Primary and Confirmatory Screen

PBMCs were isolated from human blood using Histopaque (Sigma, catalogue number 10771) and Accuspin tubes (Sigma, catalogue number A7054) in accordance with the manufacturer's instructions. Cells were resuspended in TEXMacs media. IL-2 (100 IU/ml) and TransAct T cell activation beads (1:100 dilution) were added to the PBMC and cells incubated in a humified incubator at 37° C. with 5% CO₂ for two days. PBMCs were transduced with BCMA vector, BCMA-030, with a MOI of 2.4 or TnMUC1 vector, MB-037, with a MOI of 5 and incubated at 37° C. with 5% CO₂ for two days. Cells were maintained in TEXMacs media and IL-2 at 100 IU/ml throughout the culture period. Cells were harvested 12 days after transduction and frozen in CryStor CS5 freezing media at 1×10⁸ cells/ml. Untransduced T cells were generated as a negative control. T cells were generated from one donor.

Transduction efficiency for BCMA CAR-T cells was determined by measuring binding to BCMA-AF647 using flow cytometry (MACSQuant Analyser 10). Transduction efficiency for TnMUC1 CAR-T cells was determined by measuring LNGFR expression using a PE conjugated anti-LNGFR Ab and flow cytometry (MACSQuant Analyser 10). Data was analysed using FlowJo v10.1.

### Plasma Membrane Protein Array

### Pilot Study

Donor T cells were added to slides of fixed untransfected HEK293 cells and HEK293 cells overexpressing BCMA, known T cell interactors, and control proteins. The level of binding of the BCMA specific CAR-T cells, relative to background, allowed selection of a suitable donor for the preliminary, primary, and confirmatory screen.

A positive control TnMUC1 peptide was spotted onto slides in a serial dilution, starting with neat peptide. Peptide was detected with a mouse anti-human MUC1 mAb, clone 5E5, followed by detection with an Alexa Fluor 647 conjugated anti-mouse IgG (H+L) antibody.

| TnMUC1 and MUC1 peptide sequences | Name |
|---|---|
| GV-T(AcNH-a-Gal)-S(AcNH-a-Gal)-APD-T | TnMUC1 |
| (AcNH-a-Gal)-RPAPGS(AcNH-a-Gal)-T | |
| (AcNH-a-Gal)-APPAH-amide (SEQ ID NO: 88) | |
| GVTSAPDTRPAPGSTAPPAH-amide 2trifluoroacetic | MUC1 |
| acid salt (SEQ ID NO: 89) | |

### Pre-Screen Study

Untransduced and CAR transduced T cells were added to slides of fixed untransfected HEK293 cells and HEK293 cells overexpressing BCMA, known T cell interactors, and control proteins.

### Primary Screen

For the primary screen, 4070 proteins encoding full-length human plasma membrane proteins were individually expressed in human HEK293 cells using reverse transfection. The cells were arrayed in duplicate across 13 microarray slides and fixed. An expression vector, pIRES-hEGFR-IRES-ZsGreen1, was spotted in quadruplicate on every slide to ensure a minimal threshold of transfection efficiency had been achieved or exceeded. The minimal threshold of 1.5 had previously been determined as a mean pIRES-hEGFR-IRES-ZsGreen1 signal over background.

Untransduced and CAR transduced T cells were labelled with a Cell Tracer Red dye and were used in the plasma membrane protein array at a pre-optimised ratio of T cells to HEK293 cells.

### Confirmatory Screen

Vectors encoding the hits identified in the primary screen were spotted in duplicate and used to reverse transfect human HEK293 cells. Cells were fixed and subsequently spotted with positive and negative MUC1 peptides. Duplicate slides were set up. Untransduced and transduced T cells from donor 90928 (3.2×10⁷ cells per slide), or anti-human MUC1 Ab (10 μg/ml), were applied to the plasma membrane protein array. Anti-human MUC1 binding was detected with an alexafluor 647 conjugated anti-mouse IgG (H+L) antibody.

### Data Analysis

Flow cytometry: Data was analysed using FlowJo v10.1

Plasma membrane protein array: Binding was assessed by imaging for fluorescence and quantitated for transduction efficiency using ImageQuant software (GE). Levels of background binding were determined using areas of untransfected HEK293 cells.

A protein ‘hit’ was defined as duplicate spots showing a raised signal compared to background levels. This was achieved by visual inspection using the images gridded on the ImageQuant software. Hits were classified as ‘strong, medium, weak, or very weak’ depending on the intensity of the duplicate spots.

### Results and Discussion

### Generating CAR-T Cells to Support Pilot Study

BCMA-AF647 was used to determine the transduction efficiency of BCMA CAR-T cells. On day 7 of the transduction and expansion process the transduction efficiency for donors 12021, 30865 and 90928 were 85.5%, 80.3% and 69.6%, respectively (Table 17). On day 14 of the transduction and expansion process the transduction efficiency for donors 12021, 30865 and 90928 were 62.2%, 50.6% and 55.8%, respectively.

TABLE 17
Generating CAR-T cells to support pre-screen

| | | Percent positive | Percent positive |
|---|---|---|---|
| | | BCMA-AF647 | BCMA-AF647 (%) |
| Donor | Cells | (%) on day 7 | on day 14 |
| 12021 | Untransduced | 0.22 | 0.37 |
| | BCMA transduced | 85.5 | 62.2 |
| 30865 | Untransduced | 0.29 | 0.93 |
| | BCMA transduced | 80.3 | 50.6 |
| 90928 | Untransduced | 0.25 | 1.03 |
| | BCMA transduced | 69.6 | 55.8 |

### Generating CAR-T Cells to Support Primary and Confirmatory Screen

Transduction efficiency was determined 12 days after transduction. The transduction efficiency of BCMA CAR-T cells was 63.1% (Table 18). Using the same gating strategy applied to transduced T cells, 0.8% of untransduced T cells fell within the positive gate. Using an anti-LNGFR Ab, the transduction efficiency of TnMUC1, MB-037, CAR-T cells was 29.2%. Using the same gating strategy applied to transduced T cells, 1.4% of untransduced T cells fell within the positive gate.

TABLE 18
Generating CAR-T cells to support
primary and confirmatory screen

| | Percent positive | Percent positive |
|---|---|---|
| Cells | BCMA-AF647 (%) | LNGFR (%) |
| Untransduced T cells | 0.8 | n/a |
| BCMA CAR-T cells | 63.1 | n/a |
| Untransduced T cells | n/a | 1.4 |
| TnMUC1 CAR-T cells (MB- | n/a | 29.2 |
| 037) | | |

### Plasma Membrane Protein Array: Pilot Study

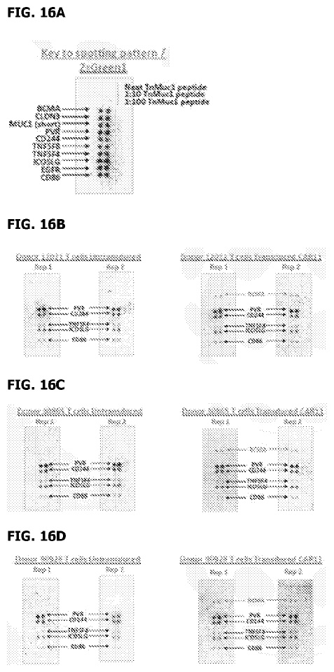

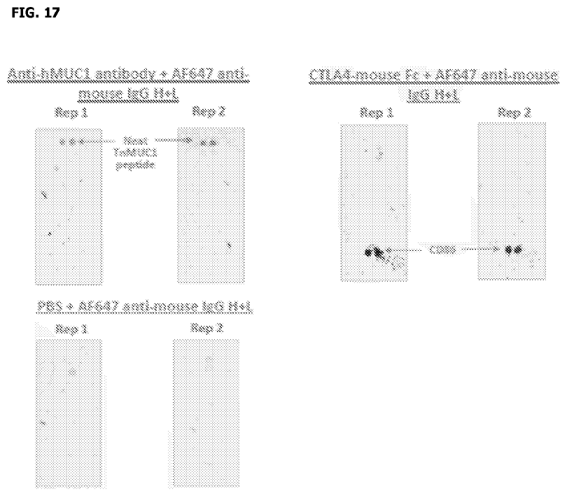

The spotting pattern for HEK transduced cells is shown in FIG. 16A. Binding was observed with untransduced T cells to known T cell interactors (PVR, CD244, TNFSF4, ICOSLG, CD86) (FIGS. 16B-D). Binding was observed with BCMA transduced T cells to BCMA transfected HEK293 cells (FIG. 17). Intensity of binding was comparable between the three donors.

### Plasma Membrane Protein Array: Pre-Screen

Donor 90928 was selected for the primary screen.

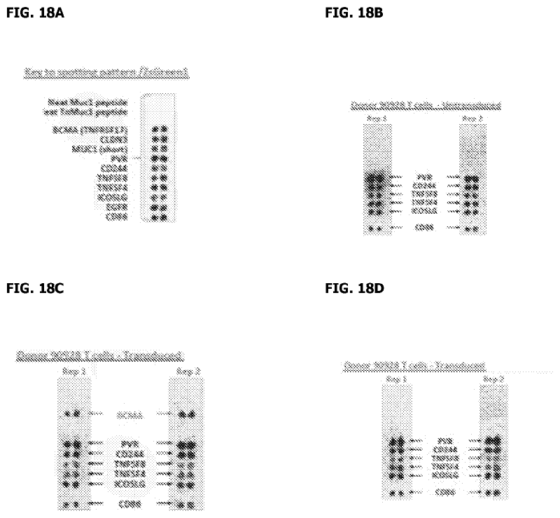

The spotting pattern for HEK transduced cells is shown in FIGS. 18A-18D. Binding was observed with untransduced T cells to known T cell interactors (PVR, CD244, TNFSF4, ICOSLG, CD86). Binding was observed with BCMA transduced T cells to BCMA transfected HEK293 cells. No binding occurred with TnMUC1 transduced T cells to TnMUC1 or MUC1 peptide.

### Plasma Membrane Protein Array: Primary Screen

In total 28 hits were identified by analysing fluorescence on ImageQuant. The intensity of staining ranged from very weak to strong.

### Plasma Membrane Protein Array: Confirmatory Screen

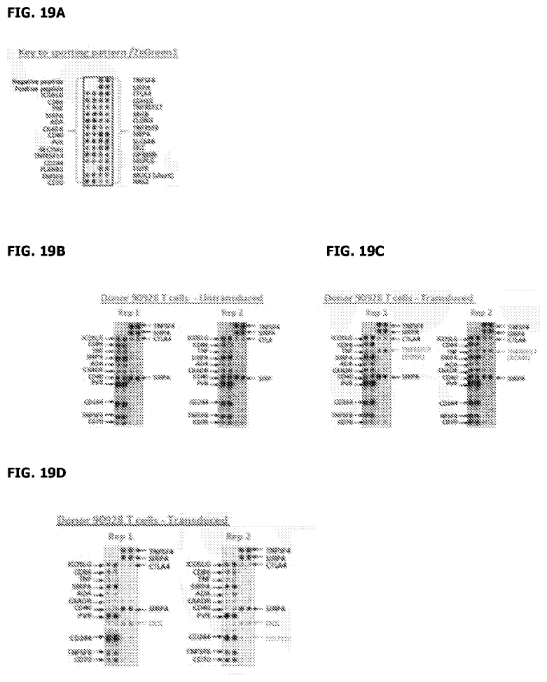

The spotting pattern for the 28 hits is shown in FIGS. 19A-19D. Binding was observed with untransduced T cells to known T cell interactors. One specific interaction was identified for BCMA CAR-T cells with strong intensity. Two CAR-specific interactions were identified for TnMUC1 CAR-T cells. These were DCC netrin 1 receptor (DCC) (medium intensity) and Selectin P ligand (SELPLG) (very weak/weak intensity).

In sum, after screening the CAR-T cells for binding against human HEK293 overexpressing full library of 4070 human proteins, the untransduced T cells showed binding to many known T cell interactors. BCMA CAR-T cells, used as positive control, showed a single specific interaction with BCMA with strong intensity. The TnMUC1 CAR-T cells showed binding to DCC netrin 1 receptor with medium intensity and Selectin P ligand with very weak/weak intensity. No binding was observed between TnMUC1 CAR-T cells to TnMUC1 or MUC1 peptide. Binding to DCC netrin 1 receptor was further evaluated since medium intensity binding was observed (see Example 16). The binding to selectin P ligand was very weak/weak which is deemed as a low confidence binder, so this protein will not be further evaluated.

### Example 7—Impact of CAR Expression on T Cell Phenotype

The objective of this study was to use a 12-colour phenotyping panel to characterise the phenotypic status of seven humanised TnMUC1 CAR-T cells in the basal status (unchallenged) 14 days after transduction, by comparing to untransduced T (UT) and murine versions of T cells. The tested humanised TnMUC1 CAR T cells included HuCAR020-26 and murine CAR T cells included MB004 (mouse version positive TnMUC1 CAR T) and MB007 (mouse version negative TnMUC1 CAR T that is lacking signalling domains 4-1BB and CD3z). The designed 12-colour phenotyping panel includes lineage (CD3 and CD8), activation/exhaustion checkpoints (LAG3, TIM3 and PD1), memory subsets (CD45RA and CCR7) and CAR transduction (zsGreen) biomarkers. We have employed a machine-learning platform Cytobank with computational data analysis algorithms to analyse the generated high-dimensional flow data. Parallel data analysis was performed using FlowLogic.

### Methods

### Experimental Preparation(s)

Purified T cells were prepared from a precursor experiment with donor numbers 91860, 91462 and 92091 (All the transduced CAR T cells have zsGreen gene expression in the construct). Briefly, T cells (14 days post transduction) were cultured in 6-well cell culture plates with TexMACS medium plus 100U of IL-2. After an overnight incubation, unchallenged T cells were harvested, and each sample was normalised to the same transduction efficiency (TE) of 32% using UT T cells for comparison purpose prior to the staining. The tested T cells included untransduced T (UT), MB004 (murine version positive TnMUC1 CAR T), MB007 (mouse version negative TnMUC1 CAR T that is lacking signalling domains 4-1BB and CD3z) and MB020-26 (humanised version positive TnMUC1 CAR Ts).

### Experimental Protocol(s)

### Fc Blocking and Cell Surface Staining

Human IgG with stock concentration of 5 mg/mL was used as Fc blocker (1:50 dilution). T cells were washed with DPBS twice before Fc receptor blockage with the addition of 40 uL of excess irrelevant Human IgG Isotype Control diluted 1:50 in BD Brilliant Stain Buffer and incubated for 15 minutes at room temperature. Cells were then stained with 40 μL of a 2× concentrated antibody cocktail containing all antibody-fluorochrome conjugates diluted in BD Brilliant Stain Buffer. Cells were Incubated with antibody cocktail for 30 minutes in the dark at room temperature. Cells were then washed twice with DPBS before the addition of 100 μL of viability dye Zombie NIR diluted 1:2000 in DPBS. Following a 15-minute incubation of cells with viability dye at room temperature in the dark, cells were washed a further two times with DBPS before being read on a BD LSRII flow cytometer.

### Cytometer Set-Up & Compensation

CS&T beads were used daily to evaluate cytometer performance and inform accurate application settings for aligned acquisition of data across each timepoint. Compensation was calculated prior to the acquisition of sample data in FACSDiva using Invitrogen Ultra Comp eBeads stained with each antibody fluorochrome conjugate.

### Data Analysis

Flow cytometry data was analysed using Flowlogic 7.2.1 software and Cytobank 7.0 platform to produce primary metrics and plots. Activation status was defined as CD69+41BB+co-expressing T cells. Exhaustion status was defined as LAG3+ TIM3+PD1+co-expressing T cells. Memory subsets were defined as Stem cell memory/Naïve (Tscm/Naïve: CD45RA+,CCR7+), effector memory (Tem: CD45RA−,CCR7−), central memory (Tcm: CD45RA+,CCR7−) and terminally differentiated effector memory (Temra: CD45RA−,CCR7+). Data was normalised to account for donor to donor variability by comparing all conditions within each donor to MB007 (non-signalling CAR control). GraphPad Prism 7 was used to graph metrics representing results from 3 donors. Statistical analysis was applied within GraphPad Prism using a One-way ANOVA and a Dunnett's multiple comparisons test.

### Results and Discussion

### Transduction Efficiency (TE) and CD4+/CD8+ Ratio

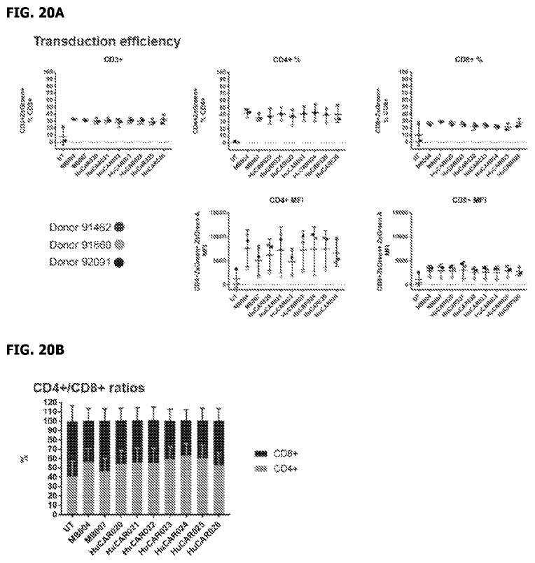

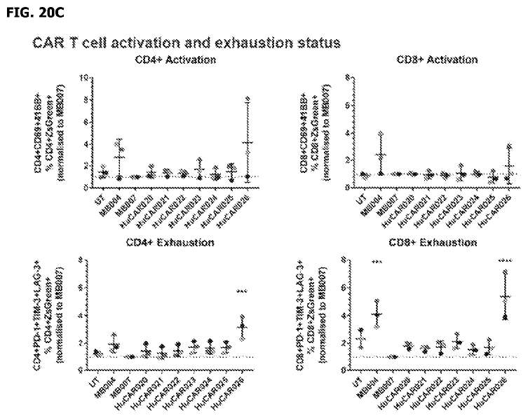

Based on the percentage of zsGreen positive cells, CAR-T samples are normalised using UT T cells to an overall TE of ˜32% in all donors (n=3) (FIG. 20A). However, the CD4+ T cell subsets have higher TE (˜30-50%), compared to that of CD8+ T cell subsets (˜20-30%) with donor-to-donor variability. The degree of positivity for these transduced T cells are also highly donor dependent, especially for CD4+ T cell subsets. In general, the CD4/CD8 ratio is also donor dependent and UT and MB007 CAR T cells have more CD8+ subsets, compared to all other CAR-T samples (FIG. 20B).

### Analysis of Activation and Exhaustion Status of T Cells

In this study, a 12-colour flow cytometry panel was employed to evaluate the basal phenotypes of 10 products with 3 donors, namely 7 humanised TnMUC1 CAR T constructs (MB020-26), mouse TnMUC1 CAR-T (MB004), non-specific CAR-T control (MB007) & untransduced T cells (UT). The activation status is defined by cells co-expressing CD69 and 41BB, and the exhaustion status is defined by cells co-expressing PD1, LAG3 and TIM3. Overall, on the basal level, there are low activation as well as exhaustion status for all samples, except for MB004 and MB026 (FIG. 20C). The overall trend shows that on the basal level, MB004 and MB026 T cells appeared to have relatively higher activation (CD69+ 41BB+) and exhaustion (PD-1+LAG-3+ TIM-3+) status, and MB023 T cells show moderately high status, compared to all other samples, possibly indicating high self-activation/early burnout trend.

### Analysis of Memory Subsets of T Cells

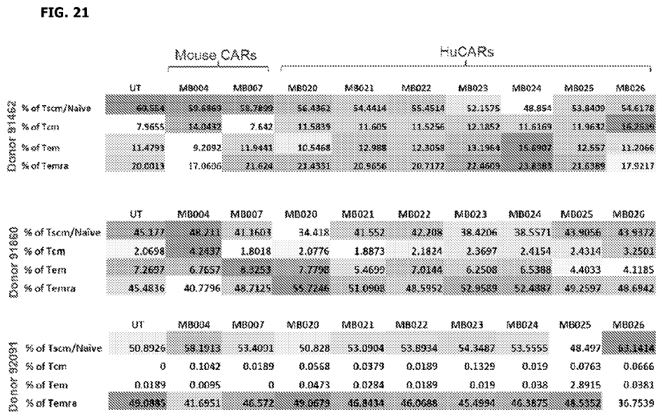

On the basal level, there are subtle donor-to-donor T memory subsets variations. This can be revealed by conventional flow cytometry gating analysis with manual gating using fluorescence minus ones (FMOs). In terms of percentages, there is no major difference of memory subsets for untransduced and transduced T cells in all samples, except Donor 92091 hardly has any Tem and Tcm subsets in all untransduced and transduced samples. All three donors showed that humanised CAR MB004 and MB026 T cells have slightly higher percentages of Tscm/Naïve (and Tcm) subsets (FIG. 21).

Despite the high donor to donor variability, it was observed that MB004 and MB026 generally have a relatively higher level of activation (CD69+41BB+), compared to all other humanised CAR T cells. They also exhibited significantly higher level of exhaustion (PD-1+LAG-3+ TIM-3+), this may indicate a high self-activation/early exhaustion situation of these CAR T constructs.

### Example 8—Tonic Signalling (Antigen Independent Signalling) for Humanised TnMUC-1 CAR-T Cells

The objective of this study was to evaluate the effect of tonic signalling (antigen independent signalling) for humanised TnMUC-1 CAR-T cells in-vitro. CAR-T cells that exhibit tonic signalling lead to impaired in vitro T-Cell function and exhaustion and inferior in vivo efficacy. Tonic signalling is influenced by a combination of features of the CAR structure, linker or hinge, signalling domains, surface expression location and levels. The humanised TnMUC-1-BBζ(PGK) CAR-T cells studied in this Example constitute of a humanised 5E5 ScFv, a 4-1BBζ cytosolic domain without IgG1 CH2-CH3 linker generated using a lentivector transduction with a PGK promoter and LNGFR detection motif.

### Methods

### Experimental Preparations

### CAR-T Cell Thawing and Culture

Where cryo-frozen CAR-T cells were used, the cells were semi-thawed in a water-bath set at 37° C. and resuspended with 1 mL of cold TEXMACS media under aseptic conditions in a safety cabinet, until the pellet had fully thawed. The cell suspension volume was adjusted to a total volume of 10 mL in a 15 mL FALCON tube using cold TEXMACS media and centrifuged at 300×g for 5 minutes at room temperature (RT). The supernatant was removed, and the retained cell pellet resuspended in 10 mL of TEXMACS media twice.

For resuspension and culture of thawed or fresh cells, the resulting cell pellet post washing was resuspended in 2 mL TEXMACS media at RT and the cell density obtained using the cell counter. The cells were resuspended, and the density was adjusted to 2×10⁶ cells/mL in TEXMACS media with 100 U/mL IL-2. 7 mLs of the resuspended cells were transferred into respective wells of a 24 deep well G-Rex plate and placed in a humidified incubator for 24 hrs at 37° C. with 5% CO₂ prior to LNGFR enrichment.

### CAR-T Cell LNGFR Enrichment

LNGFR expressing CAR-T wells were positively selected using the EASYSEP Human CD271 Positive Selection Kit and EASYSEP Dextran RAPIDSPHERES. The CAR-T cells were harvested in 15 mL Falcon tubes and centrifuged at 300×g for 5 min at RT, the supernatant removed. The cell pellet was resuspended to a density of 10 to 20×10⁶ cells in 200 μL of TEXMACS medium supplemented with 5 μL of EASYSEP Human FcR Blocker and 10 μL of EASYSEP Human CD271 Positive Selection Cocktail provided in the assay kit and transferred to a U-bottom non-tissue culture treated 96-well plate and incubated for 15 minutes at RT. 10 μL of EASYSEP Dextran RAPIDSPHERES were added to respective wells containing the cell suspension and incubated for 15 minutes at RT. The cell suspension was resuspended by addition of 60 μL wash buffer (dPBS (without calcium and magnesium) containing 2% Foetal Bovine Serum (FBS) and 2 mM EDTA) and the plate was placed on to the EASYPLATE EASYSEP Magnet and incubated for 10 minutes. The cell supernatant was carefully removed using a multichannel pipette without disturbing the cell pellet and the wash cycle repeated four times using 200 μL of wash buffer. After the last wash, the cells were resuspended in 200 μL of TEXMACS medium supplemented with 10 U/mL of human IL-2 and transferred to each well of a G-Rex plate containing 6.5 mLs of TEXMACS medium supplemented with 10 U/mL of human IL-2 and placed in a humidified incubator for 24 hrs at 37° C. with 5% CO₂ prior to subsequent assays.

### Lysate Generation and Protein Quantification

Lysis buffer was prepared by dissolution of 1 tablet of cOmplete™ Stop and PhosSTOP™ in 1 mL of cold RIPA buffer and stored on ice.

CAR-T and un-transduced T-cells were harvested from cultures into 15 mL Falcon™ tubes and the cell density acquired using a cell counter. Volumetric equivalent of 2×10⁶ cells was transferred into another 15 mL Falcon™ tube. The cells were centrifuged at 300×g for 5 minutes at RT and the supernatant removed. The cell pellet was resuspended in 1 mL of cold dPBS (with calcium and magnesium) and transferred into 1.5 mL Eppendorf™ tubes. The cells were centrifuged using the Eppendorf™ microfuge at 2000 RPM for 5 minutes at 4° C. and the supernatant removed. Residual supernatant was removed using a 100 μL pipette. The resulting cell pellet was lysed by repeat pipetting of 70 μL of cold lysis buffer at 20-minute intervals over a period of 1 hour. The lysates were centrifuged at 13,500 RPM for 5 minutes at 4° C. and 20 μL aliquots snap frozen on dry ice. Aliquots can be stored at −80° C. for long term storage.

The protein level in the lysates was quantified using the BSA assay in which BSA was titrated at the following concentrations: 0, 25, 125, 250, 500, 750, 1000, 1500, and 2000 μg/mL. 20 μL of the BSA titration was transferred in duplicate and a single sample of the cell lysates was transferred into a 96 Flat-bottom well polystyrene NUNC plate. Working reagent was prepared by diluting 200 μL of the BCA reagent B with 10 mL of reagent A diluent. 200 μL of the working reagent was added to respective wells containing either the BSA or the cell lysate. The plate was shaken using the multidrop plate shaking option for 30 seconds and incubated at RT for 2 hours prior to reading on the CLARIOSTAR plate reader.

### Experimental Protocols

### Determination of Cytokine Release in CAR-T Cell Supernatants

CS&T beads were used to evaluate cytometer performance. Compensation was calculated prior to the acquisition of sample data in FACS Diva using Invitrogen Ultra Comp eBeads stained with each antibody fluorochrome conjugate. CAR-T and un-transduced T-cells were harvested from cultures into 15 mL Falcon tubes and centrifuged at 300×g for 5 minutes at RT. 50 μL of the supernatant from each sample was aliquoted into a 96 well V-Bottom assay plate.

BD™ CBA human Th1/Th2 cytokine kit II and BD™ CBA human granzyme B flex set D7 were combined into one multiplex assay and used to determine IL2, IL4, IL6, IL10, IFNγ, TNFα & granzyme-B. Lyophilized assay standards were reconstituted in 1 mL of cell culture media and 100 μL of the reconstituted samples used to conduct a 1 in 2, 11-point serial dilution conducted to resolve the calibration plot. 50 μL of cell culture media was used to define the background cytokine levels.

A bead mix consisting of 4 μL of each of the CBA human Th1/Th2 cytokine kit II capture beads and 0.5 μL of the CBA human granzyme B flex set D7 capture beads for each test sample well was prepared in a 2 mL microcentrifuge tube and vortexed. Similarly, a PE detection reagent master mix was prepared by addition of 0.5 μL of CBA human granzyme B flex set D7 PE detection reagent to 25 μL of CBA human Th1/Th2 cytokine kit II PE detection reagent for each test sample well in another 2 mL microcentrifuge tube. 25 μL of the capture beads master mix and 25 μL of the PE detection reagent master mix were added to 50 μL of supernatant and to 50 μL of assay standards respectively. The assay plates were sealed and incubated at RT in the dark on a plate shaker set at 600 rounds per minute (RPM) for 3 hrs.

The assay plate was centrifuged at 300×g for 5 minutes and the supernatant removed. The beads were washed with 100 μL of CBA Human Th1/Th2 Cytokine Kit II wash buffer twice and resuspended in 60 μL wash buffer prior to acquiring samples on the BD LSRII cytometer.

### Determination of CAR-T Cell Phenotype

CAR-T and un-transduced T-cells were harvested from cultures into 15 mL Falcon tubes and the cell density acquired using a cell counter. 2×10⁵ cells were taken and centrifuged at 300×g for 5 minutes at room temperature, the supernatant removed, and cells washed in dPBS. Following a repeated centrifugation and removal of supernatant, Fc receptors were blocked by adding excess of irrelevant anti-human IgG Isotype Control diluted 1:50 in BD Brilliant Stain Buffer. Fc receptor blockage was carried out in 40 μL for 15 minutes at room temperature. Cells were subsequently stained with 40 μL of a 2× concentrated antibody cocktail containing all antibody-fluorochrome conjugates diluted in BD Brilliant Stain Buffer. Cells were incubated with antibody cocktail for 30 minutes in the dark at room temperature. Cells were then washed twice with DPBS and incubated in 100 μL of viability dye Zombie Aqua (1:2000 dilution in DPBS) for 15 minutes at room temperature in the dark. Cells were washed a further two times with DBPS before being read on a BD LSRII flow cytometer.

CS&T beads were used to evaluate cytometer performance. Compensation was calculated prior to the acquisition of sample data in FACS Diva using Invitrogen Ultra Comp eBeads stained with each antibody fluorochrome conjugate.

### Determination of Downstream Signalling (Peggy-Sue™)

Assay reagents and ladder was prepared by reconstitution of dithiothreitol (DTT) provided in assay kit with 40 μL of milliQ water. The biotinylated ladder was diluted with 16 μL of milliQ water, 2 μL of sample buffer, 2 μL of reconstituted DTT and 16 μL transferred to a 250 μL PCR tube. Fluorescent marker was diluted with 20 μL of DTT and 20 μL of sample buffer. 3 μL aliquots of the fluorescent marker were dispensed into 250 μL PCR tubes.

Cell lysates were diluted to 370 μg/mL using RIPA buffer and 12 μL aliquots were transferred to PCR tubes containing 3 μL of fluorescent marker. The fluorescent lysate mixtures and the biotinylated ladder were vortexed then centrifuged using the Eppendorf™ microfuge at 2000 RPM and placed on a thermocycler heat block set at 95° C. for 5 minutes. The samples were cooled on ice and 10 μL of the biotinylated ladder and fluorescent cell lysates aliquoted into respective wells of the 384 well Peggy-Sue™ assay plate as defined by the plate map.

15 μL pCD3ζ, pZAP70 and total IκBα, 3 μL of total CD3ζ, pERK1/2 and 0.3 μL of GAPDH antibodies were diluted to a final volume of 300 μL using sample buffer. 150 μL of Luminol™ was diluted into 150 μL of peroxide.

20 μL of diluted antibodies, Luminol™-Peroxide mix, milliQ water, separation matrix, stacking matrix, HRP conjugated streptavidin and anti-rabbit secondary antibody were aliquoted into respective wells as defined by the plate map. The 384 well assay plate was centrifuged at 1000×g for 1 minute to remove air bubbles before loading on the Peggy-Sue™ machine.

### Reagents and Materials

### PBMC Donors Used for CAR-T Cell Generation

CAR-T cells and un-transduced T-cells were derived from peripheral blood derived mononuclear cells (PBMCs) from 6 healthy human donors (#92205, 90774, 92084, 90244, 92190 and 92192). The CAR-T cell tested in the assay are detailed in Table 19.

TABLE 19
CAR-T Reference Numbers and Construct Components

| CAR-T Cell | CAR | Signalling | Transduction | Detection | Assay |
|---|---|---|---|---|---|
| Reference | Reference | Domain | LV Promoter | Marker | Purpose |
| MB037 | TnMUC1-BBζ | 4-1BBζ | PGK | LNGFR | Test CAR-T |
| | (PGK) | | | | Cell |
| MB039 | TnMUC1-BBζ | 4-1BBζ | PGK | LNGFR | Test CAR-T |
| | (PGK) | | | | Cell |
| MB040 | TnMUC1-BBζ | 4-1BBζ | PGK | LNGFR | Test CAR-T |
| | (PGK) | | | | Cell |
| MB041 | TnMUC1-BBζ | 4-1BBζ | PGK | LNGFR | Test CAR-T |
| | (PGK) | | | | Cell |
| MB049 | CD19-BBζ | 4-1BBζ | PGK | LNGFR | Negative |
| | (PGK) | | | | Control |
| MB059 | GD2-BBζ | 4-1BBζ | PGK | LNGFR | Test Control |
| | (PGK) | | | | |
| MB060 | GD2-28ζ | CD28ζ | EF1a | LNGFR | Test Control |
| | (PGK) | | | | |
| MB061 | GD2-BBζ | 4-1BBζ | PGK | LNGFR | Test Control |
| | (EF1a) | | | | |
| MB062 | GD2-BBζ | CD28ζ | EF1a | LNGFR | Positive |
| | (EF1a) | | | | Control |

### Data Analysis

Flow cytometry data analysis was conducted using Flowlogic 7.2.1 software producing primary metrics and plots. CD4±& CD8+ T cell subsets were compared for the expression and co-expression of activation and exhaustion markers CD69, TIM3 and PD1.

CBA data analysis was conducted using Flowlogic 7.2.1 software producing primary metrics (median fluorescence intensity values). GraphPad Prism 7 was used to calculate standard curves using a polynomial: second order (Y=A±B*X±C*X\hat{ }2) non-linear curve fit model. Interpolation from standard curves was used to determine concentrations of supernatant cytokine in pg/mL.

Antigen Independent Signalling data analysis was conducted using Compass for SW software (Peggy-Sue™) producing primary metrics. Here, the Area under Peak (AuP) for respective stains was determined using the software and the responses normalized based on AuP of GAPDH (total protein load) levels.

For phospho-CD3ζ, the normalization involved:

$$Normalised pCD 3 ζ = ( \frac{pCD 3 ζ}{Total pCD 3 z} ) / GAPDH$$

Data from two separate experiments comprising of a maximum of 6 donors was consolidated. The Bonferroni one-way ANOVA analysis was conducted to show statistical differences between the test CAR-T and controls CAR-T cells. GraphPad Prism 7 was used to graph all data representing results from up to 6 donors.

### Results and Discussion

After LNGFR enrichment and 24-hour recovery culture, a transduction efficiency of 80% was achieved across all CAR-T cells from 6 donors. Tonic signalling (antigen independent signalling) for TnMUC-1-BBζ (PGK) CAR-T cells was assessed, wherein the responses were benchmarked versus CD19-BBζ (PGK) (MB049) and GD2-28ζ (EF1a) (MB062) CAR-T cells. The level of tonic signalling was determined by a collection of assays including: basal level of cytokines in supernatants (IFNγ, TNFα and Granzyme B), differentiation of continuous T-cell phenotype by measuring activation (CD69) and exhaustion (PD-1 and LAG-3) markers, and measurement of enhanced antigen independent signalling (pCD3ζ, pZAP70, pERK and IκBα).

The level of CAR expression on the T-cells was estimated from the total-CD3ζ staining. GD2 CARs expressed using lentivector transduction with the EF1a promoter conferred a higher level of staining compared to CARs expressed using PGK promoter. Lower levels of phospho-CD3ζ, cytokine release and differentiation in activation and exhaustion phenotype was observed on the GD2-28ζ (PGK) (MB060) CAR-T cells compared to the same construct on the EF1a promoter. This reaffirms the efficiency of the vector can induce tonic signalling by over expression of the CAR (Gomes-Silva et al., 2017). GD2-BBζ (PGK and EFIa) (MB059 and MB061) CAR-T cells did not show any increase in tonic signalling compared to the CD19-BBζ (PGK) (MB049) CAR-T cells. However, GD2-28ζ (PGK and EF1a) (MB060 and MB062) CAR-T cells showed an increase in tonic signalling compared to the GD2-BBζ (PGK and EF1a) (MB059 and MB061) CAR-T cells. The tonic signalling effect was further augmented on the GD2-28ζ (EF1a) (MB062) CAR-T cells. The data reaffirms the 4-1BBζ cytosolic domain decreased the level of tonic signalling compared to the CD284 transmembrane and cytosolic domain independent of the lentivector transduction promoter used (Long et al., 2015). However, the GD2-28ζ CAR-T cell includes an IgG1 CH2-CH3 extracellular linker which is not present on the GD2-BBζ CAR-T cell and could also contribute to the level of tonic signalling observed (Frigault et al., 2015) (Mamonkin et al., 2016).

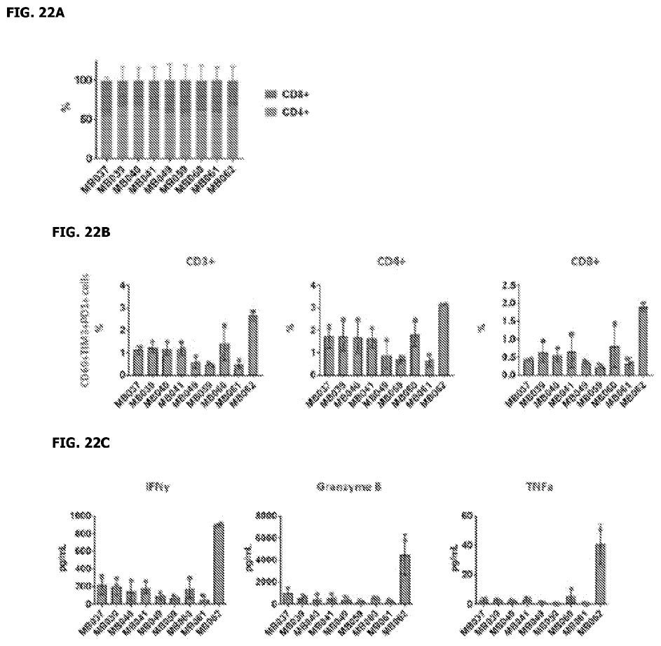

In general, there were slightly more CD4+ T-Cells compared to CD8+ T-cells across 3 donors sampled in this assay (FIG. 22A). CD4+ T-cells showed a higher expression of combined activation (CD69) and exhaustion (PD-1, TIM-3) phenotype compared to CD8+ T-cells, a feature which was consistent across all CAR-T cells. The trend for the activation and exhaustion phenotype was retained when investigating triple positive (CD69, PD-1 and TIM-3), double positive exhaustion only (PD-1, TIM-3) or PD-1 only profiles (FIG. 22B.).

The cytokine release (IFNγ, TNFα and Granzyme B) and the activation (CD69) and exhaustion (TIM-3, PD-1) of the test TnMUC-1-BBζ (PGK) CAR-T cells show a profile akin to that observed for the CD19-BBζ (PGK) (MB049) and the GD2-BBζ (PGK and EF1a) CAR-T (MB059, MB061) cells with no tonic signalling. A slight increase in cytokine release, activation and exhaustion phenotype for the TnMUC-1-BBζ(PGK) and GD2-28ζ (PGK) (MB060) CAR-T cells was observed. However, this cytokine release, activation and exhaustion phenotype were significantly lower than that observed for the GD2-28ζ (EF1a) (MB062). This data indicates minimal level of tonic signalling observed for all four humanised TnMUC-1-BBζ (PGK) CAR-T cells.

All the TnMUC-1-BBζ (PGK) CAR-T cells (MB037 to MB041) conferred a lower activation and exhaustion phenotype compared to the GD2-28ζ (EF1a) CAR-T cells (MB062). The activation and exhaustion phenotype observed for the TnMUC-1-BBζ (PGK) CAR-T cells and GD2-28ζ (PGK) CAR-T cells (MB060) was comparable. However, the activation and exhaustion phenotype for the humanized TnMUC-1-BBζ (PGK) showed a slight increase compared to un-transduced T-cells, the CD19-BBζ (PGK) (MB049) or the GD2-BBζ (PGK and EF1a) CAR-T cells (MB059, MB061) (FIG. 22B).

From the basal cytokine release profiles, the TnMUC-1-BBζ (PGK) and GD2-28ζ (PGK) (MB060) CAR-T cells conferred significantly lower levels (Bonferroni one-way ANOVA) of IFNγ, Granzyme B and TNFα compared to the GD2-28ζ (EF1a) (MB062). The CD19-BBζ (MB049) and GD2-BBζ (PGK and EF1a) (MB059, MB061) CAR-T cells conferred least detection of basal cytokine release (FIG. 22C).

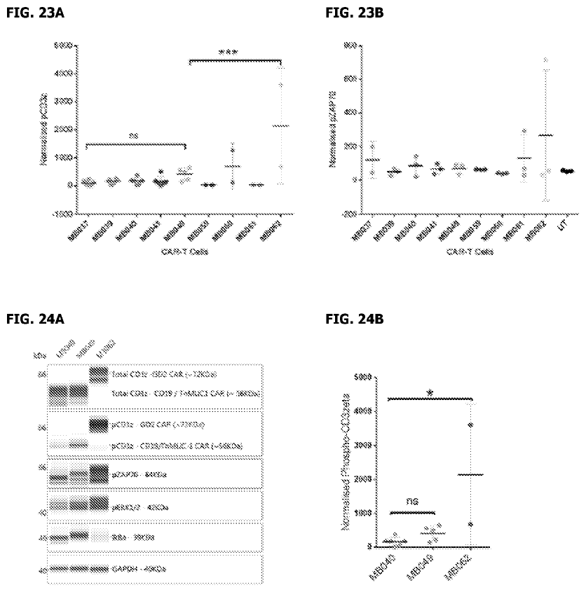

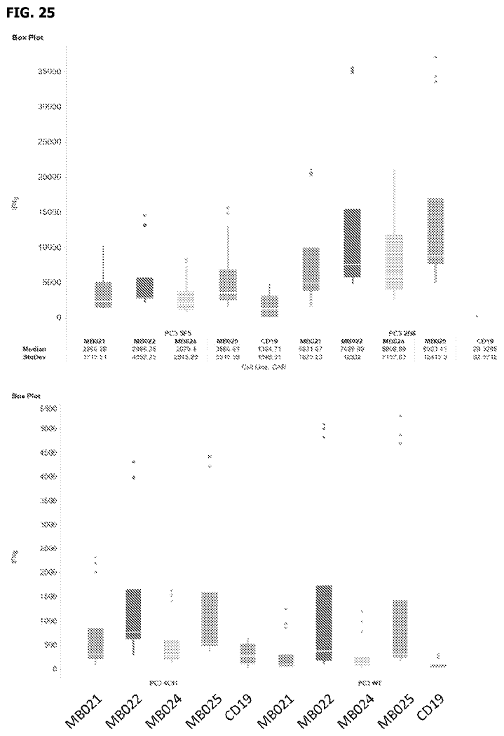

Optimisation of downstream T-cell signalling detection was conducted by cross linking of the T-cell receptor and measuring the kinetic increase in pCD3z, pZAP70, pERK and a reduction in total IκBα peaking within the first few minutes. Downstream signalling analysis of the GD2-28ζ (EF1a) CAR-T cell (MB062) conferred an increase in pCD3ζ and pZAP70, pERK and a reduction in IκBα compared to TnMUC-1-BBζ (PGK), CD19-BBζ (PGK) (MB049), GD2-BBζ (PGK and EF1a) and GD2-28ζ (PGK) CAR-T cells (FIG. 23A and FIG. 24A. From the signalling profile detected on the Peggy-Sue™ Western assay, the humanised Tn-MUC-1-BBζ (PGK) and GD2-28ζ (PGK) (MB060) CAR-T cells conferred significantly lower levels of pCD3ζ compared to the GD2-28ζ (EF1a) (MB062). The pCD3ζ levels were similar across all the humanised TnMUC-1-BBζ (PGK) CAR-T cells in line with the negative control CD19-BBζ (PGK) (MB049). This data reaffirms insufficient level of tonic signalling for the TnMUC-1 CAR-T cells to adversely affect CAR-T cell function in-vitro.

Lysates were obtained from 2×10⁶ CAR-T cells and the concentrations normalized to 370 μg/mL prior to loading. The protein levels were assessed using the Peggy-Sue™ high throughput capillary western technology. The normalized level of pCD3ζ were calculated based on total-CD3ζ and GAPDH loading control from a maximum of 6 donors. From the results analysis, the humanized TnMUC-1-BBζ (PGK) CAR-T cells (MB037 to MB041) conferred no significant difference in pCD3ζ or pZAP70 levels compared to the CD19-BBζ (PGK) (MB049) CAR-T cell. However, significantly higher levels of pCD3ζ were detected for the GD2-28ζ (EF1a) compared to the TnMUC-1-BBζ (PGK) CAR-T cells. Low pCD3ζ signal was observed for the GD2-BBζ (PGK or EF1a) (MB059 and MB061) CAR-T cells (FIG. 23A). Similar analysis for pZAP70 confirmed highest level of signal for the GD2-28ζ CAR (EF1a) (MB062) and no significant differences between the TnMUC-1-BBζ (PGK), CD19-BBζ (PGK) (MB049) or GD2-BBζ (PGK or EF1a) CAR-T cells (FIG. 23B).

Downstream signal transduction mapping onto the TCR signalling pathway was determined for the TnMUC-1-BBζ CAR (MB040), CD19-BBz CAR (MB049) and the GD2-28ζ (EF1a promoter) CAR (MB062) CAR-T cells. Here, the level of CAR specific total-CD3ζ, pCD3ζ, pZAP70, pERK1/2, total IκBα were established for CAR-T cells obtained from donor 90244. As depicted from the western blot (FIG. 24, A), the GD2-28ζ CAR (MB062) showed a significant increase in total-CD3ζ, pCD3ζ, pZAP70 and pERK1/2 with a reduced level of total-IκBα compared to the CD19-BBζ (MB049) and TnMUC-1-BBζ (MB040) CAR-T cells.

### Example 9—Cytotoxicity of Humanised TnMUC1 CAR-T on TnMUC1 Tumour Cell Lines

The aim of the study was to assess the cytotoxicity and specificity of TnMUC1 CAR-T in killing assays, when co-cultured with positive and negative TnMUC1 tumour cell lines. Specifically, CAR-T cell cytotoxicity was measured in real time in xCELLigence assay over a 72 hr time course. In addition, activation of CAR-T cells was assessed by measuring IFNγ release using MSD assay after 24 hr of co-culturing with tumour cells.

### Methods

Functionality of TnMUC1 CAR-T was assessed by co-culturing of TnMUC1 CAR-T with tumour cell lines. CAR-T cells were used at day 14 post transduction. Direct cytotoxicity was measured in real-time with xCELLigence assay, tumour cells were seeded 20 hrs prior to T-cell addition. Co-cultures were run in xCELLigence for 72 hrs with two positive TnMUC1 cell lines, MDA-MB-468 and MCF7 WT. In addition, CAR-T efficacy was assessed by measuring level of secreted IFNγ at 24 hrs post-culture with K562, MCF7 WT and MCF7 MUC1 KO cell lines. Experiments were set up with three donors in two separate sets of experiments.

### Experimental Preparation(s)

### T-Cell Preparation

T-cells were harvested from a 24-well G-REX plate at day 14 post-transduction. T-cells were counted and diluted to 1×10≢cells/ml in complete RPMI media. T-cells were centrifuged at 300×g for 5 min and washed in 10 ml complete RPMI media and centrifuged again at 300×g for 5 min. T-cells were resuspended at 1×10⁶ cells/ml. CAR-T were normalised to lowest transduction efficiency across donors. Transduction efficiency was based on % positive ZsGreen population from flow cytometry analysis. Normalisation was performed by dilution with untransduced (UT) T-cells to a fixed number and volume of T-cells.

### Experimental Protocol(s)

xCELLigence Cytotoxicity Assay

The xCELLigence RTCA instrument (ACEA Biosciences) was applied for this impedance experiment. Each well of a 96 well E Plate (ACEA Biosciences) was filled with 50 μl of target cell culture media so that the background impedance could be measured prior to target cell addition. Target cells MDA-MB-468 and MCF7 WT were dissociated and seeded at a density of 20,000 cells/well (MDA-MB-468 and MCF-7 WT). 50 μl of each cell line were added to the appropriate wells of a 96 well E Plate. After the target cells were seeded, the E-plates were left at room temperature for 15-30 minutes to allow target cells to adhere to the wells. E Plates were then transferred to the RTCA instrument (inside a cell culture incubator) and data recording was initiated straight away at 1-hour intervals for the experiment time course. Approximately 20 hours post seeding, data acquisition was paused, and effector cells were added at 0.2:1 CAR-T to Target Ratios for MDA-MB-468 and MCF-7 WT cell line. The controls present were Target Cell only, effector cells only and Target plus 100% Lysis (0.5% Triton X) wells. E Plates were then placed back into the instrument and the experiment resumed. Additional co-cultures were set up in the same conditions for cytokine analysis.

| Cell Line | Seeding density (cells/well) |
|---|---|
| MDA-MB-468 | 20000 |
| MCF7 | 40000 |
| MDA-MB-468 | 30000 |
| MCF7 | 20000 |

### Human IFNγ Cytokine Assay

K562, MCF7 WT and MCF7 KO cell lines were detached and counted. Cells were washed in RPMI media and centrifuged at 300×g. Cells were resuspended at 1×10⁶ cells/ml. 5×10⁴ cells were then added to the 96-well plate, in 100 ul. 100 ul of normalised T-cells were then added to the plate at 2.5:1 E:T ratios (effector is based on transduced T-cells) and co-cultured at 37° C., 5% CO₂ for 24 hrs. After 24 hrs plates were centrifuged, supernatants were collected and stored at −80 degrees. For MSD analysis samples were thawed at room temperature. Samples and calibrators were diluted in Diluent 1. Specifically, 10 μl of the stock calibrator was diluted with 990 μl of diluent 1. A 1:4 serial dilution was used to prepare the 6 additional calibrator dilutions. Diluent 1 only was used at the final dilution. Samples were diluted 1:10 in diluent 1 and 25 μl of each sample were added to the MSD plate. Calibrators were added in duplicate in the first two columns of the plate. Plates were sealed and incubated at room temperature with shaking for 2 hours. Plates were washed three times with PBS containing 0.5% Tween (Sodexo) using the plate washer. Detection antibody was diluted in diluent 100 (60 μl of Ab+2.94 mL diluent 100 per MSD Plate). Following the addition of 25 μl of detection antibody, the plates were sealed and incubated at room temperature with shaking for 2 hours. Plates were washed as before. Then, 150 μl of 2×read buffer was added to each well before reading on the MSD Sector 600 Imager. Data was analysed using excel and prism software. Briefly, the experimental values corresponding to the amount of released IFN-γ coming from the co-culture.

### Data Analysis

xCELLigence

Changes in impedance were recorded as Cell Index (CI). Cell Index was normalised to the point of effector cell addition—known as Normalised Cell Index (NCI). The equation below was used to calculate % viable cells and % cytolysis via normalisation of the data to target cell growth in Microsoft Excel.

Normalisation to Target (with Lysis)=(((coculture-effector)−100% Lysis)/(Target−100% Lysis))*100

### MSD

Data was analysed using excel and prism software. Briefly, the experimental values corresponding to the amount of released IFNγ coming from the co-culture have been calculated by the linear interpolation (the absorbance values have been interpolated to calculate the concentration). The regression curve used increasing concentration (pg/mL) of: IFNγ (Top Value: 10.000 pg/mL).

### Results and Discussion

Differences in cell line glycolysis and cell line biomarkers can result in differential killing between cell lines. MUC1 can be differently glycosylated by Tn or sTn and this will affect the rates and amount of killing between different cell lines. The xCELLigence assay showed that the rate and overall percentage of killing seen was different between the MDA-MB-468 and MCF7 cell lines. No major differences can be seen between CAR-T within a donor by xCELLigence cytotoxicity. MB024 could efficiently target TnMUC1 positive cells at comparable levels to both the MB004 mouse and MB020 human positive control CAR-T.

### Evaluation of MB024 Cytotoxicity on TnMUC1 Tumour Cell Lines

Six humanised TnMUC1 CAR-Ts were evaluated for efficacy and specificity in cytotoxicity assays. Validation of TnMUC1 CAR-T cytotoxicity was measured in real time by xCELLigence assay, where cell growth is traced over time using impedance measurements. Two TnMUC1 expressing breast cell lines (MCF7 and MDA-MB-468) were targeted by six TnMUC1 CAR-T, and cytotoxicity measured every hour for a total of 72 hrs, where lack of impedance correlated with tumour cell killing. Due to over confluency of the control target cells, cytotoxicity analysis was only valid up to 35 hr. Donor 0277 had an unusual cross reaction of the UT cells, so this donor was excluded from analysis, resulting in total of 5 donors. Although the percentage of cells alive at 72 hrs for MB024 differed by up 20% across donors, no significant differences were found for cytotoxicity between TnMUC1 CAR-T cells within a donor.

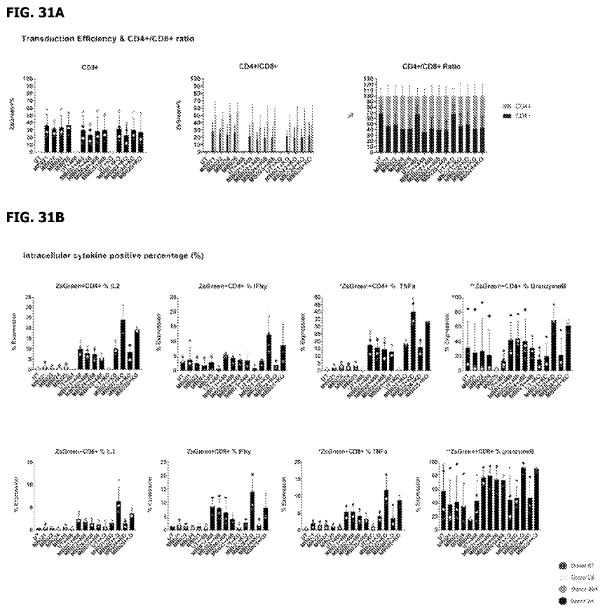

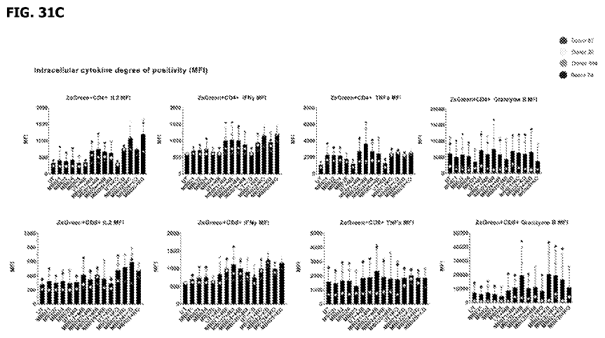

MB024 had equivalent KT50 values to other CAR-T tested on MDA-MB-468 cells. Results with three donors (91031, 0277, 91666) on the MCF7 cells were omitted as the MCF7 cell lines did not grow to a suitable density so did not pass assay criteria and excluded from the analyses, only donors 92091, 91860 and 91462 on the MCF7 cells were considered for analysis (FIG. 31, B). MB024 had slower KT50 on the MCF7 cells in comparison to the MDA-MB-468, however there was less donor to donor variability on the MCF7 cell line when compared to the killing on the MDA-MB-468 cells.

Summary tables of TnMUC1 CAR-T kinetics measuring time to reach 50% killing (KT50) are shown below in Table 20, with average KT50 on MDA-MB-468 cell line (a), and average KT50 on MCF7 WT cells (b).

TABLE 20
Killing kinetics (KT50) of TnMUC1 CAR-T cells in xCELLigence assay

| | | | | | | | |
|---|---|---|---|---|---|---|---|
| MB004 | MB020 | MB021 | MB022 | MB023 | MB024 | MB025 | MB026 |

(a) KT50 values ThMUCI cytotoxicity on MDA-MB-468 cell line

| | | | | | | | | |
|---|---|---|---|---|---|---|---|---|
| 92091 | 12.3 | 14.1 | 16.3 | 14.2 | 9.6 | 10.7 | 10.9 | 7.6 |
| 91860 | 49.9 | 21.0 | 21.0 | 18.4 | 18.7 | 16.6 | 17.3 | 9.5 |
| 91462 | 12.7 | 14.6 | 14.6 | 12.0 | 11.9 | 8.5 | 8.3 | 6.4 |
| 91031 | 9.4 | 21.7 | 17.6 | 41.7 | 14.7 | 24.1 | 16.0 | 9.4 |
| 91666 | 10.4 | 32.9 | N/A | N/A | 19.1 | N/A | 21.5 | N/A |
| Mean 5 Donors | 18.9 | 20.9 | 17.4 | 21.6 | 14.8 | 15.0 | 14.8 | 8.2 |

(b) KTS0 values ToMUC1 cytotoxicity on MCF7 cell line

| | | | | | | | | |
|---|---|---|---|---|---|---|---|---|
| 92091 | 23.7 | 24.8 | 26.7 | 23.6 | 21.9 | 29.2 | 24.9 | 23.4 |
| 91860 | 28.4 | 23.5 | 25.3 | 26.5 | 22.9 | 28.1 | 30.1 | 19.7 |
| 91462 | 20.2 | 23.7 | 24.3 | 23.7 | 24.3 | 24.6 | 24.8 | 15.3 |
| Msan 3 Donors | 24.1 | 24.0 | 25.6 | 24.6 | 23.0 | 27.3 | 26.6 | 19.5 |

*N/A - did not reach 50% killing

### Evaluation of T-Cell Activation by Assessing IFNγ Cytokine

Similar to the data seen on xCELLigence the results show that MB024 TnMUC1 CAR-T secrete IFNγ when co-cultured with TnMUC1 positive cells, indicating T-cell activation as a result of CAR-T interaction with target antigen. Activation of MB024 by targeting TnMUC1 was measured by IFNγ release after co-culture with K562, MCF7 WT and MCF7 MUC1 KO cell lines. MB024 showed T-cell activation upon interaction with TnMUC1 positive cell lines and when compared to UT and anti-CD19 CAR-T controls. MB024 did not show any level of activation on the MCF7 KO, unlike MB022, MB023, MB025 and MB026, that were activated in the absence of MUC1 on the MCF7 MUC1 KO cell line. In addition, when looking at the basal level of IFNγ release, MB024 showed no IFNγ secretion while MB026 had a high basal level of activation.

Thus, the IFNγ release of the CAR-T on the MCF7 cell line indicates that MB024 is superior to other CAR-Ts tested. All CAR-T across 6 donors show activation when cultured on the MCF7 MUC1 KO cell line, except for MB021 and MB024. MB024 also showed no level of basal IFNγ release in the absence of target cell lines. When considering the potential cross-reaction proteins for the TnMUC1 CAR-T, it is possible that all the CAR-T molecules may recognise Tn and sTn glycans on other glycoproteins present on the cell line even in the absence of the MUC1 protein. Given that Tn-glycan is only expressed in tumours this raises less of a concern due to its tumour specificity however sTn-glycan is expressed in normal tissue at low level and possible cross-reaction of the CAR-T would indicate an unfavourable safety profile. Taken together MB024 can target and efficiently kill TnMUC1 tumour cell lines and is specific to TnMUC1 positive cell lines.

### Example 10—Impact of Target Density

The aim of the study was to assess the cytotoxicity and specificity of TnMUC1 CAR-T in xCELLigence and MSD assays. Using recombinant cell lines expressing different levels of TnMUC1 we differentiated the CAR-Ts based on differential cytotoxic mediated killing. Cytotoxicity was measured in real time in xCELLigence assay over a 72 hr time course, and by measuring IFNγ release by MSD from 24 hr co-cultures.

### Methods

Functionality studies of TnMUC1 CAR-T were carried out by establishing co-cultures of TnMUC1 CAR-T with TnMUC1 positive and negative tumour cell lines. After thawing CAR-T cells were allowed to recover for 24 hrs before addition to tumour cell lines. MSD and xCELLigence plates were set up in parallel using the same cell lines and E:T ratios. Experiments were set up with four donors in two separate sets of experiments.

### Experimental Preparation(s)

### T Cell Thawing & Revival

1 mL aliquots of frozen T cells were semi-thawed at room temperature and then transferred into 15 ml of ice-cold cell culture media. T cells were centrifuged at 300×g for 10 minutes at room temperature and resuspended in 15 ml of cold cell culture media. After repeated centrifugation, T cells were resuspended in 2 mL room temperature cell culture media. T cells were counted, and cell concentration was adjusted to 2×10⁶ cells/ml. T cells were cultured in media containing IL-2 at 100 U/mL in 24 well, flat bottom cell suspension plates for 24 hrs at 37° C. with 5% CO₂ in a humidified incubator.

Coating E Plate with CD40 Antibody for Testing Suspension Cell Lines in xCELLigence

The tethering solution was diluted 10× with tissue culture grade water. This tethering solution was then used to dilute the anti-CD40 antibody from 500 μg/mL to achieve a final concentration of 4 μg/mL (125-fold dilution). The appropriate wells of the E plates (ACEA Biosciences) were coated with 50 μl of the anti-CD-40 working solution, incubated at room temperature for 3 hours, washed twice with PBS (−/−) and incubated at 1 hour with 50 μl target cell culturing media for 1 hour before taking a background measurement.

### Experimental Protocol(s)

xCELLigence Cytotoxicity Assay

The xCELLigence RTCA instrument (ACEA Biosciences) was applied for this impedance experiment. Each well of a 96 well E Plate (ACEA Biosciences) was filled with 50 μl of target cell culture media so that the background impedance could be measure prior to target cell addition. Adherent target cells were dissociated and seeded, along with the suspension cell line at densities of 20,000 (all PC3 cell lines and ARH 77 WT) or 40,000 (MCF-7 WT and MCF-7 KO) cells/well. 50 μl of each cell line was added to the appropriate wells of a 96 well E Plate. After the target cells were seeded, the E-plates were left at room temperature for 15-30 minutes to allow target cells to adhere to the wells. E-Plates were then transferred to the RTCA instrument (inside a cell culture incubator) and data recording was initiated straight away at 10-minute intervals for 6 hours, and then at 1-hour intervals for the remainder of the experiment. Approximately 20 hours post seeding, data acquisition was paused, and effector cells were added at a 1:1 Effector to Target Ratios for PC3 cells lines and ARH77 WT and added at a 0.5:1 Effector to Target Ratio for MCF-7 WT and MCF-7 KO. The controls present were Target Cell only, effector cells only and Target plus 100% Lysis (0.5% Triton X) wells. E Plates were then placed back into the instrument and the experiment resumed. Additional co cultures were set up in the same conditions for cytokine analysis. xCELLigence data was analysed as described above in Example 9.

### Human IFNγ Cytokine Assay

During the xCELLigence coculture set up, duplicate plates were set up in a flat bottomed 96 well plate and incubated for 24 hours. After centrifugation of the plates, supernatants were collected 24 hours post co culture and stored at −80° C. until thawed at room temperature for the MSD.

Samples and calibrators were diluted in Diluent 1. 10 μl of the stock calibrator was diluted with 990 μl of diluent 1. A 1:4 serial dilution was used to prepare the 6 additional calibrator dilutions. Diluent 1 only was used at the final dilution. Samples were diluted 1:10 in diluent 1 and 25 μl of each sample were added to the MSD plate. Calibrators were added in duplicate at opposite sides of the plate. Plates were sealed and incubated at room temperature with shaking for 2 hours. Plates were washed 3× with PBS+0.5% Tween (Sodexo) using the plate washer. Detection antibody was diluted in diluent 100 (60 μl of Ab+2.94 mL diluent 100 per MSD Plate). Following the addition of 25 μl of detection antibody, the plates were sealed and incubated at room temperature with shaking for 2 hours. Plates were washed as before. 150 μl of 2×read buffer was added to each well before reading on the MSD Sector 600 Imager. MSD data was analysed as described above in Example 9.

### Statistical Calculations

A linear mixed effects model was used with an interaction term for CAR by Cell Line, and random intercept terms for week, plate and donor, for each of 24, 48 and 72 hours separately. Using these models, linear contrasts were calculated for ‘difference in % cell alive vs negative control cell line’ using the following formula:

Difference in % alive=(CAR-T response in cell line−UT response in cell line)−(CAR response in control cell line−UT response in control cell line)

### Results and Discussion

### Functional Evaluation of TnMUC1 Cytotoxicity on TnMUC1 Tumour Cell Lines

The levels of TnMUC1 cell surface expression can vary dramatically in endogenously expressing cell lines. By using recombinant cell lines that keep the level of expression constant between experiments, it enables accurate experimental repeats when using different donors for CAR-T supply. By using a range of target levels, CAR-Ts could potentially be differentiated by kinetics and IFNγ measurements.

The xCELLigence cytotoxicity assay was used to study the ability of TnMUC1 CAR-T cells to differentially target and kill tumour cells expressing TnMUC1 at different levels. Cytotoxicity was measured in real time every hour post T-cell addition, for 72 hours. In this assay three recombinant PC3 tumour cell lines expressing TnMUC1 at high (100%), medium (60%) and low (5%) percentage positivity were tested with their PC3 WT negative control. MCF7 WT and MCF7 MUC1 KO cell lines were also used as experimental controls. We showed that the TnMUC1 PC3 5F5 cell line exhibited targeted cytotoxicity within 2 hours of T-cell addition, with significant killing seen for only MB022 at 24 hours. No killing can be seen at 24 hours across the other cells when compared to the control cell lines.

At 48 hours all CAR-T show some level of killing on the MCF7 WT, PC3 2B6 and PC3 5F5. However, MB021 is the only CAR-T that has significant killing on the PC3 2B6. While the MB022 and MB025 show significant killing on the MCF7 WT at this 48-hour time point, and MB021 and MB024 do not. Despite the different E:T target ratios between MCF7 and PC3 cell (0.5:1 and 1:1 respectively), by the end of the 72-hour time course all CAR-T show 100% killing on MCF7 WT, PC3 2B6 and PC3 5F5 and no killing is observed on the PC3 4C11. Although some killing is seen with MB021 on PC3 4C11 it is not statistically significant (Table 21). Overall, the most significant killing is seen on the MCF7 WT and PC3 2B6, even though these cell lines have less TnMUC1 expression than the PC3 5F5.

TABLE 21
Statistical significance of TnMUC1 CAR-T cytotoxicity on TnMUC1
positive cell lines relative to negative control cell lines

| Comparison | Reference | Comparison | p-value @24 | p-value @ | p-value @ |
|---|---|---|---|---|---|
| CAR | Cell Line | Cell Line | hrs | 48 hrs | 72 hrs |
| CD19 | MCF-7 KO | MCF-7 WT | 0.8777 | 0.5487 | 0.8777 |
| | PC3 WT | PC3 2B6 | 0.3901 | 0.4667 | 0.3901 |
| | PC3 WT | PC3 4C11 | 0.5964 | 0.4932 | 0.5964 |
| | PC3 WT | PC3 5F5 | 0.3066 | 0.3653 | 0.3066 |
| MB021 | MCF-7 KO | MCF-7 WT | p < 0.0001 | 0.1287 | p < 0.0001 |
| | PC3 WT | PC3 2B6 | p < 0.0001 | 0.0045 | p < 0.0001 |
| | PC3 WT | PC3 4C11 | 0.0983 | 0.1093 | 0.0983 |
| | PC3 WT | PC3 5F5 | 0.0036 | 0.0214 | 0.0036 |
| MB022 | MCF-7 KO | MCF-7 WT | p < 0.0001 | p < 0.0001 | p < 0.0001 |
| | PC3 WT | PC3 2B6 | p < 0.0001 | 0.1172 | p < 0.0001 |
| | PC3 WT | PC3 4C11 | 0.66 | 0.0902 | 0.66 |
| | PC3 WT | PC3 5F5 | 0.0044 | 0.006 | 0.0044 |
| MB024 | MCF-7 KO | MCF-7 WT | p < 0.0001 | 0.0829 | p < 0.0001 |
| | PC3 WT | PC3 2B6 | p < 0.0001 | 0.6198 | p < 0.0001 |
| | PC3 WT | PC3 4C11 | 0.8605 | 0.0892 | 0.8605 |
| | PC3 WT | PC3 5F5 | 0.0008 | 0.0086 | 0.0008 |
| MB025 | MCF-7 KO | MCF-7 WT | 0.0003 | p < 0.0001 | 0.0003 |
| | PC3 WT | PC3 2B6 | p < 0.0001 | 0.1295 | p < 0.0001 |
| | PC3 WT | PC3 4C11 | 0.4475 | 0.3497 | 0.4475 |
| | PC3 WT | PC3 5F5 | 0.0206 | 0.0273 | 0.0206 |

### Evaluation of T-Cell Activation by IFNγ Cytokine Release

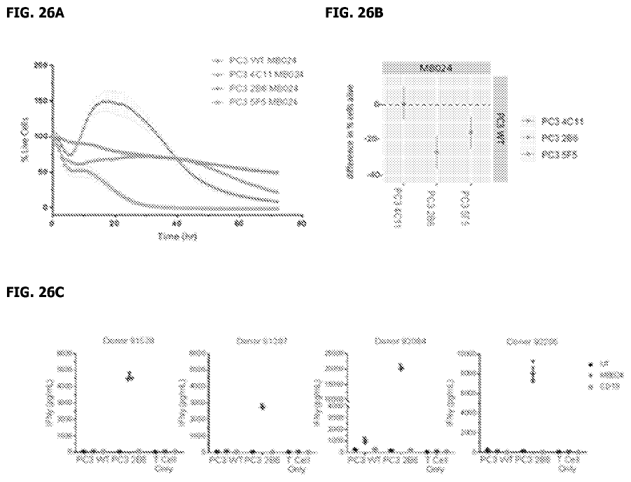

MB022 and MB024 CAR T cells secreted high level of IFNγ when co-cultured with highly positive TnMUC1 tumour cell lines. IFNγ release was highest on the PC3 COSMC KO cell line 2B6 with approximately 8000 pg/ml detected for MB021 and MB024, while MB022 and MB025 reached equivalent levels of 10000 pg/ml (FIG. 26). Similar trends were seen on the PC3 5F5 cell line, but with lower levels of IFNγ (3000 pg/ml-5000 pg/ml). MB021 and MB024 showed no activation on PC3 4C11 or PC3 WT cell line. Overall MB024 expressed 2-fold less IFNγ release in comparison to MB025, however there is a large difference in expression levels between positive and negative cells, with no activation on the PC3 WT or basal expression (FIG. 26).

The results demonstrate that there is a threshold of expression necessary for TnMUC1 CAR-T cells to specifically engage and kill their target cell line. CAR-T cells showed no cytotoxic effects in co-culture with the PC3 4C11 cell lines expressing only 5% TnMUC1 in both xCELLigence and MSD. The PC3 5F5 (high expression) and PC3 2B6 (medium expression) cell lines could be completely eliminated by MB024, slower cytolysis kinetics were observed for PC3 2B6, despite the lower levels of IFNγ released from the PC3 5F5.

### Example 11—Functional CAR-T Cell Killing of TnMUC-1 Expressing PC3 Cell Lines

The purpose of this study was to select a TnMUC-1 CAR-T cell based on its propensity to specifically kill TnMUC-1 expressing target cells.

### Methods

### Experimental Protocol(s)

### CAR-T Cell Thawing and Culture

CAR-T cells and un-transduced (UT) T-cells were derived from peripheral blood derived mononuclear cells (PBMCs) from 3 healthy human donors (#92159, 92160 and 2764). Where cryo-frozen CAR-T cells were used, the cells were semi-thawed in a water-bath set at 37° C. and resuspended with 1 mL of cold TexMACS™ media until the pellet had fully thawed. The cell suspension volume was adjusted to a total volume of 10 mL in a 15 mL Falcon® tube using cold TexMACS™ media and centrifuged at 300×g for 5 minutes at room temperature (RT). The supernatant was removed, and the retained cell pellet resuspended in 10 mL of TexMACS™ media twice.

For resuspension and culture of thawed or fresh cells, the resulting cell pellet post washing was resuspended in 2 mL TexMACS™ media at RT and the cell density obtained using the cell counter. The cells were resuspended, and the density was adjusted to 2×10⁶ cells/mL in TexMACS™ media with 100 U/mL IL-2. 7 mLs of the resuspended cells were transferred into respective wells of a 24 deep well G-Rex plate and placed in a humidified incubator for 24 hrs at 37° C. with 5% CO₂ prior to co-culture set-up.

The CAR-T cell tested in the assay are detailed below.

CAR-T cell vectors

| Name | Unique ID |
|---|---|
| LV.VSVg.SIN.PGK.H-scFv.D4.Muc1CAR.W | MB-051 |
| LV.VSVg.SIN.PGK.H-scFv.D6.Muc1CAR.W | MB-052 |
| LV.VSVg.SIN.PGK.H-scFv.D16.Muc1CAR.W | MB-053 |
| LV.VSVg.SIN.PGK.H-scFv.D18.Muc1CAR.W | MB-054 |

### Co-Culture Set-Up for Cell Killing Assay on the IncuCyte S3™

Three cell lines were tested: PC3 COSMC KO Clone 5F5 (PC3.5F5), PC3 COSMC KO Clone 4C11 (PC3.4C11), and PC3 WT (PC3.wt). Target cells (PC3.wt, PC3.5F5 and PC3.4C11) were dislodged from T175 flasks by treatment of the cell surface with 5 mL of TrypIE™ for 5 minutes at 37° C. in a humidified incubator. The flasks were lightly tapped to dislodge the cells and the TrypIE™ neutralised with 15 mL of pre-warmed cell culture media (RPMI+10% heat inactivated FBS+1% Sodium Pyruvate+1% MEM-NEAA+1% GlutaMAX). The cell suspension was collected into a 50 mL Falcon™ tube and centrifuged at 300×g for 10 minutes. The supernatant was removed at the cell pellet obtained resuspended in 10 mL of cell culture media. The cell density was obtained on the cell counter and the cell resuspended to 0.4×10⁶ cells/mL in cell culture media. 100 μL of the cells were transferred to respective wells of the assay plate as depicted by the assay plate map and transferred into a humidified IncuCyte™ S3 at 36.5° C./5% CO₂ for 24 hours prior to CAR-T cell co-culture.

Cell supernatants were removed from respective assay well and replaced with 100 μL of fresh cell culture media containing 500 nM Cytotox™ Red reagent and the plates transferred into a humidified IncuCyte™ S3 at 36.5° C./5% CO₂ prior to effector cell addition.

CAR-T and un-transduced T-cells were harvested into 15 mL Falcon™ tubes and the cell density acquired using a cell counter. The cells were centrifuged at 300×g for 5 minutes at RT and the supernatant removed. The cell pellet was resuspended in cell culture media to a density of 0.45×10⁶ cell/mL and 100 μL of the CAR-T and un-transduced T-Cells transferred to respective wells as depicted by the assay plate map. The assay plate was placed in the humidified IncuCyte™ S3 at 37° C./5% CO₂. Image acquisition was scheduled at 2-hour intervals over a 6-day time span.

### Data Analysis

### Image Analysis

Image collection representing conditions detailed below were obtained for set-up of image masks
CAR-T cell and target cell co-culture at 2 hours and 96 hours
Un-transduced T cell and target cell co-culture at 2 hours and 96 hours
Target cells only at 2 hours and 96 hours
CAR-T cell only at 2 hours and 96 hours
Un-transduced cells only at 2 hours

Image analysis was conducted to ensure specific visualisation of increase in total red area depicting target cell killing and a total red area mask generated to determine the total area (μm2/Image).

### Image Data Normalisation and Analysis

The total area for respective CAR-T and un-transduced T-cells was exported into Windows Excel. The raw data and normalised % live cells for respective CAR-T and un-transduced T-Cells were plotted as a function of time in GraphPad Prism version 6.02 for Windows, GraphPad Software to represent increase in total cluster area (μm2/Image). The normalisation was calculated as detailed below:

The Cell Killing effect for each CAR-T cell was calculated using the equation listed below:

Normalised response dependent on best CAR-T killing effect in that experiment at T=1 and T=nth read, where the equation used was:

Determine Co-Culture Background response (CBG)=Co-culture response−(Effector only+Target Only Response) a.

Determine % Live Cells={[Mean (Maximum CBG of best CAR-T-CBG^{tn})]/[Mean (Maximum CBG of best CAR-T-CBG^{t1})]}×100 b.

The percent live cells at respective endpoints for each repeat from the 3 T-Cell donors (n=9 data points) tested across the three PC3 COSMC knockout cell lines (PC3.5F5—28 hrs, PC3.4C11—44 hrs and the PC3.wt—60 hrs) was obtained. The data was plotted using GraphPad Prism version 6.02 for Windows, GraphPad Software. The Bonferroni One-Way ANOVA test was conducted to determine the significant differences between % Live Cell responses for the test CAR-T cells across the different cell lines.

### Results and Discussion

The aim of the study was to establish the threshold of target expression where CAR-T specific killing of the target cell line can be detected. A major challenge in CAR design is ensuring detection and elimination of tumour cells sparing healthy tissue. Consequently, this study should also enable the detection of any non-specific killing effects observed on the PC3.wt cell where no TnMUC-1 target expression is detected.

In this study CAR-T cells without a detection tag, consisting of a humanised 5E5 ScFv with a CD8 transmembrane and a 4-1BBζ cytosolic region were generated by a lentiviral transduction of peripheral blood derived mononuclear cells (PBMCs) and cryopreserved. The cells were re-introduced to fresh culture and the CAR-T cell viability and transduction efficiency was reassessed. Here all CAR-T cells conferred a viability of greater than 80% with a CAR transduction efficiency of greater than 80%.

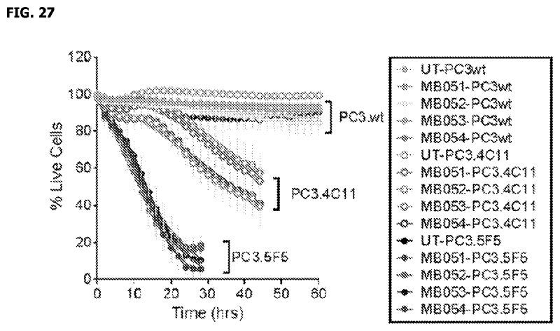

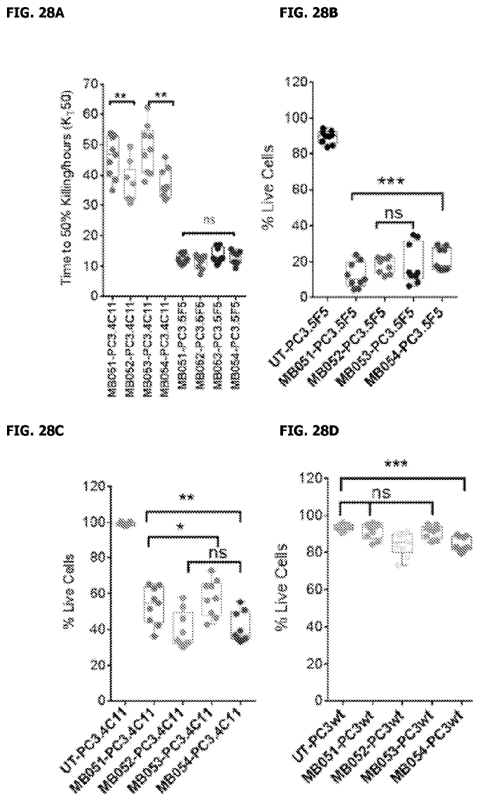

From the normalised cell killing response, the un-transduced T-cells did not show any specific killing on either of the target cell lines. MB052 and MB054 conferred some level of killing at an endpoint of 60 hrs on the PC3.wt cell line. This killing effect was not apparent on MB051 and MB053 at the same time point. Conducting a Bonferroni one-way ANOVA at the 60-hour endpoint for the test CAR-T cells showed a significant difference in killing of the PC3.wt target cell line by MB052 and MB054 CAR-T cells. At the 60-hour endpoint the average percentage live cells were lower for MB052 and MB054 compared to MB051, MB053 and un-transduced T-cells (FIGS. 27 and 28, A and B). However, the level of killing observed for MB052 and MB054 was insufficient to predict the time to kill 50% of the target cell line (Kt50) at the 60-hour endpoint.

Similarly, MB052 and MB054 also showed increased level of killing compared to MB051 and MB053 on the PC3.4C11 cell line at an endpoint of 44 hours. The Bonferroni one-way ANOVA conferred significant difference in the level of % live cells (FIG. 28C). From the Kt50 analysis MB052 and MB054 conferred a Kt50 in the range of 36-38 hours, whereas for MB051 and MB053 the Kt50 was slower with a range of 45-49 hours.

Results from testing the CAR-T cells on the PC3.5F5 cell line which confers a high level of TnMUC-1 showed significantly enhanced killing where complete killing endpoint was achieved at 28 hrs. The Bonferroni one-way ANOVA analysis on the endpoint showed little difference in the % live cells for MB051, MB052 and MB053. The only significant difference was observed between MB051 and MB054. Because of the enhance killing response, the Kt50 was much shorter compared to when testing on the low TnMUC-1 expressing PC3.4C11 cell line. Here the calculated Kt50 was in a arrange of 11-14 hours, with no significant difference between any of the four CAR-T cells (FIGS. 28A-28D).

The results obtained from the experiment where PC3.wt and COSMC knockout cell lines expressing increasing levels of TnMUC-1 confirmed a representative increased killing activity of the TnMUC-1 CAR-T cells. By using varying levels of TnMUC1 we demonstrated that there is a threshold of expression necessary for TnMUC1 CAR-T cells to specifically engage and kill their target cell line. CAR-T cells showed no or poor cytotoxic effects in co-culture with the PC3.wt well line which has no expression of TnMUC-1. MB051 and MB053 conferred no significant difference in killing over a 60-hour end point of the PC3.wt cell line. However, MB052 and MB054 showed a significantly increased level of killing compared to that observed by the un-transduced T-cells or by MB051 and MB054 on a TnMUC-1 negative cell line (PC3.wt). This observation could be related to a slow off-rate driven mechanism where the ScFv for the MB052 and MB054 conferred a slower off-rate and higher affinity compared to MB051 and MB053.

With an increase of 5% TnMUC-1 expression on the PC3.4C11 cell line there is sufficient target to enable the CAR-T cells to engage and kill. Because of the low TnMUC-1 expression levels on the PC3.4C11 cell line, the maximum threshold of killing observed compared to the PC3 5F5 (high expression) ranges between 39-50%. This level of killing is achieved over a 44-hour timepoint. MB051 and MB053 confer higher % live cell thresholds and longer Kt50 compared to MB052 and MB054 indicating a slower rate of killing on the PC3.4C11 cells (Table 22). Consequently, on the TnMUC-1 high expressing cell line (PC3.5F5) near 100% killing of the target cell is achieved in 28 hours. The CAR-T cells confer an average Kt50 in the range of 12-14 hours with no significant difference in the rate of killing in either of the clones tested (Table 23).

The data confirms the importance of understanding the interplay between target density, CAR-T cell expression and CAR affinity to drive specific tumour cell killing. Here we have shown MB051 and MB053 to present a better safety profile compared to MB052 and MB054 with differential killing of target cells depending on the level of TnMUC-1 expression.

TABLE 22
Extrapolated mean and standard deviation Kₜ50 for TnMUC-1
CAR-T cells on PC3.4C11 and PC3.5F5 cell lines

| Target Cell Line | CAR-T Cell | Mean ± Stdev Kₜ50 (hrs) |
|---|---|---|
| PC3.4C11 | MB051 | 45.93 ± 6.84 |
| | MB052 | 36.61 ± 6.68 |
| | MB053 | 48.79 ± 8.10 |
| | MB054 | 37.16 ± 4.90 |
| PC3.5F5 | MB051 | 12.42 ± 1.58 |
| | MB052 | 11.47 ± 2.22 |
| | MB053 | 13.53 ± 2.51 |
| | MB054 | 12.53 ± 2.13 |

TABLE 23
Average % live cells and standard deviation for respective
UT and CAR-T cells obtained at set thresholds - PC3.wt
(60 hrs), PC3.4C11 (44 hrs) and PC3.5F5 (28 hrs)

| | | | | |
|---|---|---|---|---|
| UT | MB051 | MB052 | MB053 | MB054 |

Target Cell: PC3.wt (60 hrs)

| | | | | | |
|---|---|---|---|---|---|
| Mean | 93.45 | 91.57 | 84.65 | 91.18 | 84.90 |
| StDev | 1.71 | 4.30 | 6.28 | 3.50 | 3.95 |

Target Cell: PC3.4C11 (44 hrs)

| | | | | | |
|---|---|---|---|---|---|
| Mean | 98.98 | 53.41 | 39.54 | 57.91 | 40.95 |
| StDev | 1.01 | 10.42 | 10.15 | 10.16 | 8.41 |

Target Cell: PC3.5F5 (28 hrs)

| | | | | | |
|---|---|---|---|---|---|
| Mean | 89.38 | 12.48 | 18.32 | 18.39 | 21.33 |
| StDev | 3.58 | 7.04 | 4.38 | 11.12 | 6.37 |

### Example 12—Peptide Stimulation Assay

The aim of this study was to (i) validate BIAcore previous findings that show that the scFv used to develop MB024 is able to bind both Tn and STn MUC1; and (ii) to demonstrate that MB024 CAR-T cells bind Tn MUC1 peptides only at the reported epitope.

### Methods

### Experimental Preparation(s)

Generation of scFv Proteins, Preparation of Peptides

The humanised scFv proteins were generated as detailed previously.

Biotinylated 20mer amino acid peptides containing Tn sugar residues at Serine and Threonine amino acids across the peptide were designed and ordered from Cambridge Research Biosciences (CRB). As a control, a non-glycosylated MUC1 peptide was also ordered with the same sequence, but with no carbohydrate residues on the Serine and Threonine amino acids. In order to determine the epitope of where the humanised anti-TnMUC1 scFv proteins bind the Tn peptide, a further two differentially glycosylated peptides were ordered, with one containing Tn sugar residues on Ser/Thr amino acids of the peptide reported in the literature to be where the scFv proteins bind, and one containing Tn sugar residues on Serine and Threonine amino acids where the scFv proteins reportedly do not bind.

Biotinylated 20mer amino acid peptides containing STn sugar residues at Serine and Threonine residues at the reported epitope in the literature were also designed and ordered from CRB.

The sequences of these peptides are detailed below and are represented in FIG. 5.

Initially, only TnMUC1 (36-2) and MUC1 (36-4) peptides only were ordered. Due to their solubility in aqueous solution being unknown, these initial peptides were reconstituted to 5 mg/ml in 75% DMSO/25% PBS. From initial experiments with the MUC1 and TnMUC1 peptides, the DMSO was not detrimental, as such large dilutions in buffer were being carried out prior to use in the assay. Therefore, for enhanced stability all remaining peptides, STnMUC1 (35-1) and the differentially glycosylated TnMUC1 peptides (96-1, 96-2) were ordered at a later date, reconstituted in 100% DMSO and aliquoted under sterile conditions. This difference in reconstitution solution did not have an effect on the peptides in the assay. All peptide aliquots were stored at −80° C. until use in experiments.

### T-Cell Preparation

A vial of frozen T cells was semi-thawed at room temperature and then transferred into 10 ml of ice-cold cell culture media. T cells were centrifuged at 300×g for 10 minutes at room temperature and resuspended in 15 ml of cold PBS. After repeated centrifugation, T cells were resuspended in 2 mL room temperature cell culture media. T cells were counted and cell concentration was adjusted to 1×10⁶ cells/ml. T cells were then added to the peptide coated plates.

### Experimental Protocol(s)

### CAR Detection at T-Cell Surface

To confirm CAR expression on T-cells, T-cells were analysed for ZsGreen expression by flow cytometry. Briefly, 100 d of T-cell sample was transferred to a 96 well V-bottom plate and analysed on a MACS Quant 10 (Miltenyi) flow cytometer.

### Peptide Assay

On day one of the assay, streptavidin high binding 96 wells plates were equilibrated to room temperature and washed 3× with 150 μl PBS/well. Peptides were prepared at a stock concentration of 2 μM in PBS for the assay, followed by a 1 in 2 serial dilution covering a concentration range of 0.5 to 0.0078 μM. The biotinylated MUC1 peptides were then added to the assay plate (100 I/well) and incubated for 1.5 hours at room temperature. Following incubation, the plates were washed 3 times as described above. At this point, TnMUC1 CAR T cells were added to the plate, at 1×10⁵ cells per well in 100 l. Plates were then incubated at 37° C. and 5% CO₂, for 24h.

On day two, plates were spun at 300 g, for 5 min. The supernatant was collected and stored at −80° C. for subsequent IFNγ analysis by MSD.

### IFNγ Production Analysis by MSD

Stored supernatants were thawed at room temperature. Samples and calibrators were prepared as per manufacturer's instructions and 25 μl of each sample were added to the MSD plate. Calibrators were added in duplicate at opposite sides of the plate. Plates were sealed and incubated at room temperature with shaking. Plates were washed 3× with PBS+0.5% Tween (Sodexo) using the plate washer. Detection antibody was diluted in diluent 100. Following the addition of 25 μl of detection antibody, the plates were sealed and incubated at room temperature with shaking for 1.5 hours. Plates were washed as before. 150 μl of 2× read buffer was added to each well before reading on the MSD Sector 600 Imager.

### Peptides

| | | | Stock |
|---|---|---|---|
| Name | Sequence | MW | Concentration |
| MUC1 peptide (non- | Biotin-[PEG2]-GVTSAPDTRPAPGSTAPPAH-amide | 2371.505 | 5 mg/ml; |
| glycosylated) | (SEQ ID NO: 77) | | 2250 μM |
| TnMUC1 peptide | Biotin-[PEG2]-GV-T(AcNH-a-Gal)-S(AcNH-a- | 3271.493 | 5 mg/ml; |
| (fully | Gal)-APD-T(AcNH-a-Gal)-RPAPG-S(AcNH-a-Gal)- | | 1500 μM |
| glycosylated) | T(AcNH-a-Gal)-APPAH-amide (SEQ ID NO: 76) | | |
| TnMUC1 peptide 1 | Biotin-PEG2-GV-TSAPD-TRPAPGS(AcNH-a-Gal)- | 2662.3 | 5 mg/ml; |
| (partially | T(AcNH-a-Gal)-APPAH-amide (SEQ ID NO: 85) | | 1878 μM |
| glycosylated) | | | |
| TnMUC1 peptide 2 | Biotin-PEG2-GV-T(AcNH-a-Gal)-S(AcNH-a-Gal)- | 2865.3 | 5 mg/ml; |
| (partially | APD-T(AcNH-a-Gal)-RPAPGSTAPPAH-amide | | 1745 μM |
| glycosylated) | (SEQ ID NO: 86) | | |
| STnMUC1 peptide | Biotin-[PEG]2-GVTSAPDTRPAPG-[Ser(Sial-AcNH- | 3360.399 | 0.8 mg/ml; |
| | a-Gal)]-[Thr(Sial-AcNH-a-Gal)]-APPAH-amide | | 238 μM |
| | (SEQ ID NO: 87) | | |

### CAR T-Cells

| T-cell | Donor |
|---|---|
| UT | 90774 |
| MB024 | |
| CD19 | |
| UT | 92187 |
| MB024 | |
| CD19 | |
| UT | 92205 |
| MB024 | |
| CD19 | |
| UT | 92804 |
| MB024 | |
| CD19 | |

### Data Analysis

### Flow Cytometry Data Analysis

Data was analysed on the MACS Quantify 2.8.1618.16380 software.

### IFNγ Data Analysis

Initial IFNγ quantification calculation was carried out on MSD Discovery Workbench 4.0.12.12 (MSD). Data was then transferred to GraphPad Prism 8.0.2 (GraphPad Software, Inc) for plot analysis.

Statistical analysis was performed in R version 3.5.1. Mixed models were fit using the Ime4 package, marginal means and linear contrasts were calculated using the emmeans package. Random intercepts were included for donor, plate and date of experiment. Fixed interaction effects were fit for concentration, treatment and peptide. In order to reduce heteroskedasticity, concentration was transformed using an inverse hyperbolic sine transform (in order to handle zeros), and IFNγ release was log 10 transformed prior to modelling. These were back-transformed to display interpretable results. Inverse hyperbolic sine transformed concentration was modelled using a natural spline with 4 degrees of freedom (determined using AIC). Linear contrasts between dose-response curves, for the difference in IFNγ release (as a fold change) between Untransduced T-cells (UT) and MB024, between each pair of peptides, are calculated at the concentrations used in the original experiment and presented as fold changes alongside FDR-corrected p-values.

### Results

### ZsGreen Transduction Efficiency Analysis

MB024 and CD19 CAR T cells were co-expressed with a ZsGreen tag, which is used for analysis of CAR expression on T-cells. Donors 90774, 92187 and 92205 showed a transduction level ranging from 25 to 30%. Donor 92804 was more highly transduced, presenting 55% for MB024 and 58% for CD19 CAR-T

To note that CD19 CAR from donor 90774 was very poorly transduced (3%). This could have been due to a technical error during transduction or during the flow assay.

### Peptide Stimulation IFN-g Activation

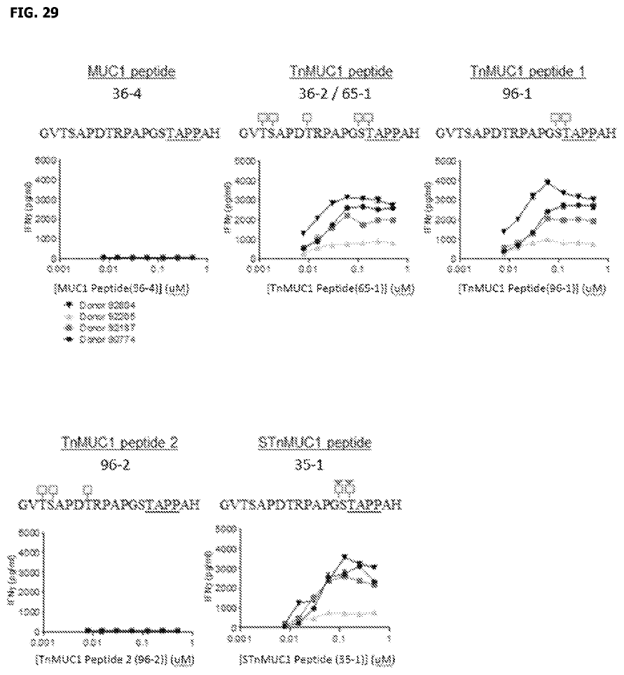

FIG. 29 shows specific IFNγ release by MB024 CAR T-cells in the presence of fully glycosylated TnMUC1 peptide (65-1), differentially glycosylated TnMUC1 peptide 1 (96-1) and STn peptide (35-1). Donor to donor variation was observed in this assay. A dose response curve was observed for each donor. The maximum IFNγ detected ranged from 1000 to 4000 μg/mL across the four different donors.

TnMUC1 CAR T-cells showed no IFNγ release when co-cultured with non-glycosylated MUC1 peptide (36-4) and the differentially glycosylated TnMUC1 peptide 2 (96-2), in which Tn sugars are present where the scFv proteins reportedly do not bind (Tarp, Sorensen et al, 2007).

Furthermore, we carried out a metanalysis for IFNγ release, with all the donors (Table 24 and Table 25). Comparing all peptides within each T-cell group (untransduced, MB024 and CAR19), shows a small difference in IFNγ release by the T-cells in the presence of STnMUC1 (35-1) vs TnMUC1 (65-1) and TnMUC1 peptide 1 (96-1). This difference was significant at a concentration of 0.015 and 0.03 μM of peptide, with a P value<0.0001 (Table 25).

P Value calculation also shows a significant difference between Tn MUC1 peptide 2 (96-2), fully glycosylated TnMUC1 peptide (65-1) and STn MUC1 (35-1) (P Value>0.0001); and no difference to unglycosylated MUC1 (36-4) (Table 24), statistically confirming that TnMUC1 peptide 2 is not able to activate CAR T-cells.

TABLE 24
IFNγ response to MB024 vs MUC1

| | | Estimate | Lower 95% | Upper 95% | P-value |
|---|---|---|---|---|---|
| Comparison | | (fold | Confidence | Confidence | (FDR |
| peptide | Concentration | change) | Interval | Interval | adjusted) |
| STnMUC1 | 0 | 0.91 | 0.736 | 1.12 | 0.54303 |
| (35) | 0.015 | 3.152 | 2.717 | 3.66 | <0.00001 |
| 35-1 | 0.03 | 5.899 | 4.981 | 6.99 | <0.00001 |
| | 0.06 | 8.252 | 6.947 | 9.8 | <0.00001 |
| | 0.125 | 9.012 | 7.498 | 10.83 | <0.00001 |
| | 0.25 | 7.152 | 5.683 | 9 | <0.00001 |
| | 0.5 | 6.613 | 5.208 | 8.4 | <0.00001 |
| TnMUC1 | 0 | 1.1 | 0.89 | 1.36 | 0.54063 |
| (65) | 0.015 | 4.955 | 4.272 | 5.75 | <0.00001 |
| 65-1 | 0.03 | 8.922 | 7.535 | 10.57 | <0.00001 |
| | 0.06 | 8.681 | 7.307 | 10.31 | <0.00001 |
| | 0.125 | 7.747 | 6.445 | 9.31 | <0.00001 |
| | 0.25 | 7.296 | 5.798 | 9.18 | <0.00001 |
| | 0.5 | 6.557 | 5.164 | 8.33 | <0.00001 |
| TnMUC1 | 0 | 0.96 | 0.777 | 1.19 | 0.88269 |
| (96-ii) | 0.015 | 1.063 | 0.916 | 1.23 | 0.59268 |
| 96-2 | 0.03 | 1.153 | 0.974 | 1.37 | 0.14791 |
| | 0.06 | 1.264 | 1.064 | 1.5 | 0.0121 |
| | 0.125 | 1.215 | 1.011 | 1.46 | 0.0581 |
| | 0.25 | 0.997 | 0.792 | 1.25 | 0.99514 |
| | 0.5 | 0.989 | 0.779 | 1.26 | 0.99184 |
| TnMUC1 | 0 | 1.201 | 0.972 | 1.49 | 0.13636 |
| (96) | 0.015 | 4.753 | 4.098 | 5.51 | <0.00001 |
| 96-1 | 0.03 | 8.403 | 7.096 | 9.95 | <0.00001 |
| | 0.06 | 8.872 | 7.468 | 10.54 | <0.00001 |
| | 0.125 | 8.199 | 6.822 | 9.86 | <0.00001 |
| | 0.25 | 7.255 | 5.766 | 9.13 | <0.00001 |
| | 0.5 | 6.611 | 5.206 | 8.4 | <0.00001 |

Data showing the P Value calculated when comparing all the peprides against non-glycosylated MUC1 peptide, for all peptide concentrations, and all donors included in the analysis.

TABLE 25
IFNγ response to MB024 vs TnMUC1

| | | | Lower | Upper | |
|---|---|---|---|---|---|
| | | Estimate | 95% | 95% | |
| Comparison | | (fold | Confidence | Confidence | P-value (FDR |
| peptide | Concentration | change) | Interval | Interval | adjusted) |
| MUC1 | 0 | 0.909 | 0.7353 | 1.124 | 0.54063 |
| (36-4) | 0.015 | 0.202 | 0.174 | 0.234 | <0.00001 |
| 36-4 | 0.03 | 0.112 | 0.0946 | 0.133 | <0.00001 |
| | 0.06 | 0.115 | 0.097 | 0.137 | <0.00001 |
| | 0.125 | 0.129 | 0.1074 | 0.155 | <0.00001 |
| | 0.25 | 0.137 | 0.1089 | 0.172 | <0.00001 |
| | 0.5 | 0.153 | 0.1201 | 0.194 | <0.00001 |
| STnMUC1 | 0 | 0.827 | 0.6689 | 1.022 | 0.11992 |
| (35) | 0.015 | 0.636 | 0.5484 | 0.738 | <0.00001 |
| 35-1 | 0.03 | 0.661 | 0.5583 | 0.783 | <0.00001 |
| | 0.06 | 0.951 | 0.8002 | 1.129 | 0.77266 |
| | 0.125 | 1.163 | 0.9679 | 1.398 | 0.1601 |
| | 0.25 | 0.98 | 0.7789 | 1.233 | 0.98774 |
| | 0.5 | 1.008 | 0.7942 | 1.281 | 0.9934 |
| TnMUC1 | 0 | 0.873 | 0.7061 | 1.079 | 0.30528 |
| (96-ii) | 0.015 | 0.215 | 0.185 | 0.249 | <0.00001 |
| 96-2 | 0.03 | 0.129 | 0.1092 | 0.153 | <0.00001 |
| | 0.06 | 0.146 | 0.1226 | 0.173 | <0.00001 |
| | 0.125 | 0.157 | 0.1305 | 0.189 | <0.00001 |
| | 0.25 | 0.137 | 0.1086 | 0.172 | <0.00001 |
| | 0.5 | 0.151 | 0.1187 | 0.191 | <0.00001 |
| TnMUC1 | 0 | 1.092 | 0.8833 | 1.35 | 0.58866 |
| (96) | 0.015 | 0.959 | 0.8271 | 1.113 | 0.79474 |
| 96-1 | 0.03 | 0.942 | 0.7953 | 1.115 | 0.68146 |
| | 0.06 | 1.022 | 0.8603 | 1.214 | 0.94377 |
| | 0.125 | 1.058 | 0.8806 | 1.272 | 0.75255 |
| | 0.25 | 0.994 | 0.7902 | 1.251 | 0.9934 |
| | 0.5 | 1.008 | 0.794 | 1.28 | 0.9934 |

Data showing the P Value calculated when comparing all the peptides against Tn MUC1 peptide, for all peptide concentrations, and all donors included in the analysis.

### Discussion

The peptide stimulation assay was designed with the aim to analyse: (i) differences in T-cell activation in the presence of Tn and STn MUC1 peptides; (ii) capability of TnMUC1 CAR to recognise random Tn positive sequences.

Data in this study shows that at very low concentrations, between 0.015 and 0.03 μM, of STnMUC1 (35-1) and TnMUC1 (65-1) there is a significant difference in IFNγ release by the MB024 CAR T-cells. This supports data from BIACore assay which shows that the scFv used to generate MB024 can bind to STnMUC1 but with less affinity than to TnMUC1. However, at higher concentrations than 0.03 μM this difference is lost, showing that beyond a certain threshold MB024 will become equally activated in the presence of Tn or STn MUC1.

Understanding the specificity of the CAR towards its target is essential for the safety of the T-cell immunotherapy. Results from the peptide stimulation assay show that MB024 CAR T-cells were not activated when co-cultured with TnMUC1 peptide 2 (96-2). This peptide contains Tn sugars in a random sequence rather than in the epitope. The next step would be to evaluate homologous sequences, positive for Tn glycosylation, to fully confirm TnMUC1 CAR specificity to Tn/STn MUC1.

In summary, this study corroborates the BIACore data and revealed that in lower concentrations of peptide, the CAR releases more IFNγ in the presence of Tn than STn MUC1. Additionally, random Tn positive sequences were not able to activate MB024.

### Example 13—Evaluating Tn/STnMUC-1 Specificity of Humanised 5E5 CAR T Cell Candidates in Co-Culture with Tn/STnMUC-1 Positive and Negative Target Cell Lines

The objective of this study was to determine target specificity of 4 humanised 5E5 CAR T cell candidates (MB021, MB022, MB024 & MB025) to cell surface Tn/STnMUC-1 by assessing CAR T cell activation when co-cultured with T cell leukaemia cell line Jurkat clone E6-1 and Jurkat's transduced with COSMC.

### Methods

### Experimental Preparations

### Humanised 5E5 CAR T Cell Generation

CAR-T cells were generated as described previously.

### Cell Line Generation

Jurkat clone E6-1 cells were modified to express COSMC gene by transducing with pG3.PGK.COSMC.IZW lentiviral vector at four MOIs (1, 3, 5 & 10). Flow cytometry was used to assess transduction efficiency and cell-surface expression of Tn/STnMUC-1. Pooled population of Jurkat's transduced with COSMC at a MOI of 10 was selected and used subsequently for co-culturing with CAR T cells as a Tn/STnMUC1 negative cell line.

### T Cell Thawing & Revival

1 mL aliquots of frozen T cells were semi-thawed at room temperature and then transferred into 15 mL of cold cell culture media. T cells were centrifuged at 300×g for 10 minutes at room temperature and resuspended in 15 mL of cold cell culture media. After repeated centrifugation, T cells were resuspended in 2 mL warm (37° C.) cell culture media. T cells were counted and cell concentration was adjusted to 2×10⁶ cells/mL. T cells were cultured in media containing IL-2 at 100U/mL in 6 well, flat bottom cell suspension plates for 24 hrs at 37° C. with 5% CO₂ in a humidified incubator.

### Cell Surface Tn/STnMUC-1, MUC-1, STn and Tn Staining for Flow Cytometry Analysis

Jurkat WT and Jurkat COSMC+ cell lines were washed with FACS buffer (DPBS+2% FBS+0.05% sodium azide) twice. 1×10⁵ cells were stained separately for Tn/STnMUC-1, MUC-1, total STn, and total Tn. Tn/STnMUC-1, MUC-1 and STn indirect staining; Staining was carried out in 50 μL of a primary antibody diluted in FACS buffer and incubated for 45 minutes at room temperature in the dark. Cells were then washed twice with FACS buffer before adding 50 μL of Brilliant Violet 421™ Goat anti-mouse secondary antibody diluted in FACS buffer and incubated for a further 45 minutes at room temperature in the dark. Cells were washed a further two times with DPBS and then resuspended in 100 μL DPBS containing Sytox Advanced viability dye diluted to 1:2000. Tn direct staining; Staining was carried out in 50 μL of antibody diluted in FACS buffer and incubated for 45 minutes at room temperature in the dark. Cells were then washed twice with FACS buffer and then resuspended in 100 μL DPBS containing Sytox Orange viability dye diluted to 1:2000. Samples were run on an LSRII cytometer. BD CS&T beads were used to evaluate cytometer performance before acquiring data.

### Experimental Protocols

### Co-Cultures Set Up

Co-cultures were set-up in flat bottom 48-well plates at a 1:1 CAR-T:target cells ratio by adding required number of CAR T cells to either 2×10⁵ Jurkat WT or 2×10⁵ Jurkat COSMC+ tumour cell lines. CAR-T:target cells ratios were calculated based on percentage transduction efficiency of CAR-T cells in which transduction efficiency was normalised across different CAR T cells (see Humanised 5E5 CAR T cell generation in experimental preparations). Co-cultures were incubated at 37° C. with 5% CO₂ in a humidified incubator for 24 hrs. 200 μL of supernatant was collected at 24 hrs and then frozen at −80° C. for subsequent cytokine measurements.

### Supernatant Cytokine Detection—Cytokine Bead Array (CBA) Assay

A BD™ CBA Human Th1/Th2 Cytokine Kit II and BD™ CBA Human Granzyme B Flex Set D7 were combined into one multiplex assay and used to analyse supernatant cytokine levels of IL2, IL4, IL6, IL10, IFNγ, TNFα & granzyme B. Assay top standards were prepared by combining lyophilised protein of each cytokine provided in each kit and reconstituting in 1 mL of cell culture media (used as assay diluent). Top standards were serial diluted 1:2 11 times in cell culture media to create assay standards ranging from 10,000-0 pg/mL. Cell culture media only was used as assay background. Supernatants were diluted 1:2 using cell culture media. A capture bead master mix was prepared by combining 4 μL of each of the CBA Human Th1/Th2 Cytokine Kit II Capture Beads (containing capture beads for all 6 cytokines) and 0.5 μL of the CBA Human Granzyme B Flex Set D7 Capture Beads (diluting granzyme B capture beads 1:50) for each test sample. A PE Detection Reagent master mix was also prepared by combining 25 μL of CBA Human Th1/Th2 Cytokine Kit II PE Detection Reagent with 0.5 μL of CBA Human Granzyme B Flex Set D7 PE Detection Reagent (diluting granzyme B PE detection reagent 1:50) for each test sample. 25 μL of the capture beads master mix and 25 μL of the PE detection reagent master mix were added to 50 μL of supernatant and to 50 μL of assay standards at the same time. Plates were sealed and incubated with shaking (600 rpm) for 3 hrs at room temperature in the dark. Plates were washed with the addition of 100 μL of CBA Human Th1/Th2 Cytokine Kit II Wash Buffer and centrifuged at 300×g for 5 minutes. Washing was repeated before resuspending in 60 μL wash buffer and running on an LSRII cytometer. BD CS&T beads were used to evaluate cytometer performance before acquiring data.

### Antibodies & Viability Dye for Flow Cytometry

| | Conjugate | | Isotype/ | | Assay | | Manufacturer |
|---|---|---|---|---|---|---|---|
| Marker | fluorophore | Reactivity | format | Clone | concentration | | and Catalog No. |
| Tn/STnMUC-1 | — | Human | Mouse, | 5E5 | 2.5 | μg/mL | Creative Biolabs |
| | | | IgG1 | | | | TAB-418MZ |
| MUC-1 | — | Human | Mouse | HMFG2 | 2.0 | μg/mL | Absolute Antibody |
| | | | IgG1/ | | | | Ab00712-1.1 |
| | | | Lambda | | | | |
| Tn | APC | Human | Mouse, | BRIC111 | 1:50 | | American Research |
| | | | IgG1, | | | | Products Inc, |
| | | | kappa | | | | 08-9414-6 |
| STn | — | Human | Mouse, | B72.3 | 0.3125 | ug/ml | Thermoscientific, |
| | | | IgG1 | | | | MS-138-P0 |
| Goat anti- | Brilliant | Mouse | Goat | Polyclonal | 0.125 | μg/mL | BioLegend 405317 |

| | | | | | | |
|---|---|---|---|---|---|---|
| mouse IgG | Violet | | Polyclonal | 4053 | | |
| Antibody | 421 ™ | | IgG | | | |
| SYTOX ™ | | | | | 1:2000 | Thermofisher |
| AADvanced ™ | — | — | — | — | | S10349 |
| Dead Cell | | | | | | |
| Stain Kit | | | | | | |
| SYTOX ™ | — | — | — | — | 1:2000 | Thermofisher |
| Orange | | | | | | |
| Dead Cell | | | | | | S34861 |
| Stain Kit | | | | | | |

### Data Analysis

Flow cytometry data was analysed using Flowlogic 7.2.1 software producing primary metrics and plots. Metrics from the Cytokine Bead Array assay were analysed in Microsoft Excel and GraphPad Prism 7. Results were graphed using GraphPad Prism 7.

GraphPad Prism 7 was used to calculate standard curves using a polynomial: second order (Y=A+B*X+C*X\hat{ }2) non-linear curve fit model. Interpolation from standard curves was used to determine concentrations of supernatant cytokine in pg/mL.

### Results

### Characterisation of Tumour Cell Lines

Jurkat WT (COSMC−) cells were 100% positive for cell surface Tn/STnMUC-1 expression compared to 1% expression on Jurkat COSMC+ cells. MUC-1 expression, as detected by HMFG2 antibody, was low in both Jurkat WT and Jurkat COSMC+ cells lines at approximately 10% and 5% respectively.

### Cytokine Responses to Tn/STnMUC-1 Positive Tumour Cells

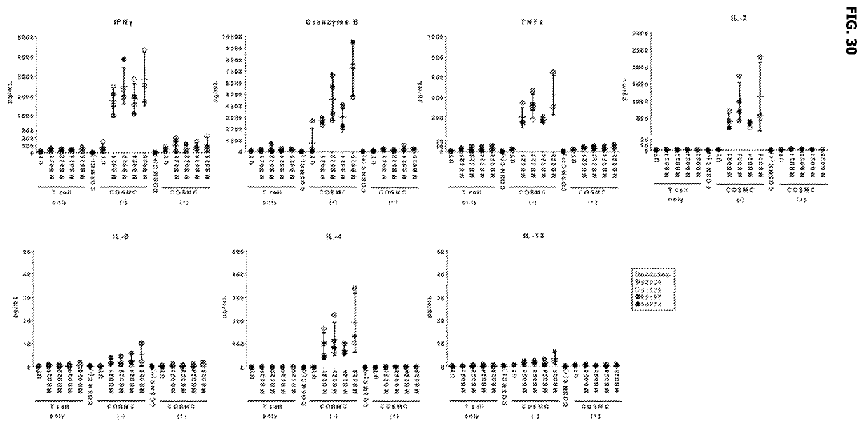

All 4 humanised CAR-T cells secreted cytokines and granzyme B in response to co-culture with Jurkat WT (COSMC−) tumour cells positive for Tn/STnMUC-1 (FIG. 30). MB022 and MB025 CAR-T cells showed a greater response compared to MB021 and MB024 CAR T cells with higher cytokine and granzyme B levels detected in supernatant after 24 hrs of co-culture. MB021 and MB024 CAR T cells share a comparable profile in the average levels of cytokine and granzyme B released. MB022 and MB025 show some similarity but in general MB025 has higher cytokine and granzyme B release compared to MB022 and the highest of all 4 CAR constructs. No statistical analysis was applied.

### Cytokine Responses to Tn/STnMUC-1 Negative Tumour Cells

All 4 humanised CAR T cells did not show cytokine or granzyme B release in response to co-culture with Jurkat COSMC+ tumour cells negative for Tn/STnMUC-1. Both cytokine and granzyme B levels detected in co-cultures with Jurkat COSMC+ were comparable to those of CAR-T cells cultured alone. No differences were observed between CAR constructs. No statistical analysis was applied.

### Discussion

All 4 humanised 5E5 CAR T cells show antigen specific reactivity to tumour cells expressing Tn/STnMUC-1. Based on cytokines assessed, all 4 CAR T cells show a strong Th1, tumour mediated response with the release of high levels of IFNγ, TNFα, IL-2 as well as granzyme B. Out of the 4 CAR constructs tested, MB025 shows the greatest overall response to antigenic stimulation producing large amounts of granzyme B in particular.

In addition to demonstrating antigen specificity, all 4 humanised 5E5 CAR T cells did not show off target reactivity to cells negative for Tn/STnMUC-1 with cytokine and granzyme B levels comparable to CAR-T cells cultured alone. Together this demonstrates all 4 humanised 5E5 CAR-T cell candidates to be both efficacious and safe within this system.

COSMC is T synthase chaperone protein, required for T synthase functionality. T synthase catalyses extension of Tn-antigen into complex 0-glycans structures typical for normal cells. The introduction of COSMC abolished endogenous Tn/STnMUC1 expression in Jurkat cells. This was mediated through the loss of Tn and STn expression as a consequence of reinstating normal cellular glycosylation. The loss of Tn/STn glycoforms and subsequent loss of Tn/STnMUC-1 expression abated activation of all 4 CAR-T cells showing specificity of these CARs is driven, in part, by Tn and/or STn recognition. Interestingly, challenge of these CAR-T cells with MCF7 MUC1 KO cells (also Tn/sTnMUC1−) showed non-specific responses of MB022 and MB025 but not MB021 or MB024 in which MB022 and MB025 produced high levels of IFNγ and granzyme B after 24 hr co-culture. Knock out of MUC-1 in MCF7 cells provides a model to study CAR specificity in which recognition of Tn/STnMUC-1 usually expressed on MCF7 WT cells is eradicated with the loss of MUC-1 expression. However, the absence of MUC-1 does not address aberrant glycosylation and the potential of Tn and STn expression on other proteins (such as other mucins). Reactivity of MB022 and MB025 to MCF7 MUC-1 KO cells could therefore be a consequence of recognising Tn or STn on other proteins other than MUC-1. This may also explain higher cytokine and granzyme B responses of MB022 and MB025 to Jurkat WT cells compared to MB021 and MB024.

In addition to the characterisation of Tn/STnMUC-1 expression on Jurkat cells, total MUC-1 expression was also determined. Total MUC-1 expression was shown to be substantially lower than that of Tn/STnMUC-1 in both Jurkat WT (COSMC−) and Jurkat COSMC+ cells. It was anticipated that total MUC-1 would be expressed at either equivalent or higher levels than Tn/STnMUC-1. However, this has been observed on consistently in Jurkats cells. HMFG2, a monoclonal antibody used to determine MUC-1 expression, derives specificity through the recognition of the MUC-1 peptide DTR motif whilst 5E5 recognises a glyco-peptide epitope specific for Tn/STn glycans on MUC-1 peptide. Discrepancies between total MUC-1 and Tn/STnMUC-1 is mostly likely related to the different binding epitopes of HMFG2 and 5E5 for MUC-1. The lack of HMFG2 binding may also be intrinsic to Jurkats as the use of HMFG2 in other cells lines was more predictable. MUC-1 clustering, conformation or peptide sequence may all be contributing factors. It has also been shown that HMFG2 binding is affected by glycosylation status of MUC1. Irrespective of this, based on the specificity of 5E5 for Tn/STn on MUC-1, we are confident that MUC-1 is expressed on Jurkat cells and aberrantly glycosylated with Tn/STn glycoforms.

Taken together, we have shown all 4 humanised 5E5 CAR T cells to be highly potent as evident by a strong Th1 response towards Tn/STnMUC-1 positive tumour cells. Moreover, all discriminate complex glycans inherent to normal tissues from tumour specific truncated Tn/STn glycoforms.

### Example 14—Polyfunctionality Evaluation of Humanised TnMUC1 CAR T Products

The objective of this study is to evaluate the ability of TnMUC1 CAR T cells to secrete a range of functional cytokines, including interleukin-2 (IL2), type II interferon (IFNγ), tumor necrosis factor alpha (TNFα) and Granzyme B (Grz B) in response to antigenic challenge. A refined protocol was designed to combine cell surface, intracellular staining (ICS) and supernatant cytokine measurement together to build a complete cytokine profile for the humanised CAR products. The ICS was used to examine what TnMUC1 CAR T cells is capable of secreting in terms of effector (GrzB, IFNγ, TNFα) and stimulatory (IL-2) cytokine expression level at 24 hr post co-culture with positive cell line MDA-MB-468 (compared with a negative cell line MCF7-KO). Cytometric Bead Array (CBA) and Luminex assays were used to correlate what the actual secreted cytokine amount of GrzB, IFNγ, TNFα and IL-2 (and IL-4,6,10 as additional cytokines for CBA assay) is in supernatant at 24 hr post co-culture with MDA-MB-468 (compared with MCF7-KO).

### Methods

### Experimental Preparation(s)

### T Cell Thawing and Revival

The tested T cells are untransduced T (UT) and HuCAR021, 022, 024 and 025 (humanised version of TnMUC1 5E5 CAR T with zsGreen gene expression in the construct) cells on Day12 post transduction. 1 mL aliquots of frozen purified humanised CAR T cells from three donors (n=3) were semi-thawed at room temperature and then transferred into 15 mL of cold cell culture media. T cells were centrifuged at 300×g for 10 minutes at room temperature and resuspended in 15 mL of cold cell culture media. After repeated centrifugation, T cells were resuspended in 2 mL warm (37° C.) cell culture media. T cells were counted and cell concentration was adjusted to 2×10⁶ cells/mL. T cells were cultured in media containing IL-2 at 100 U/mL in 6-well, flat bottom cell suspension plates for 24 hours at 37° C. with 5% CO₂ in a humidified incubator.

### Tumour Cell Plating and Co-Culturing Set-Up

Human breast carcinoma MDA-MB-468 and MCF7 MUC1 KO cells were harvested and resuspended in warm cell culture media and MDA-MB-468 and MCF7 MUC1 KO cells were plated at 1×10⁶ cells per well in 6-well plates overnight before the addition of humanised CAR T cells. For co-culturing, plates were set-up by adding untransduced T cells or transduced huCAR T cells into the 6-well plates, containing tumour cells, at an effector:target ratio of 1:1. The exact amount of T cells were calculated based on percentage of transduction efficiency of each CAR T cells, in which transduction efficiency was normalised to the lowest percentage of all four humanised CAR-T constructs by adding UT cells. Transduction efficiency is shown as % of zsGreen positive cells as determined by flow cytometry analysis. Co-cultures were incubated at 37° C. with 5% CO₂ in a humidified incubator for 24 hrs. Next day, 500 μL of supernatant was collected and then frozen in −80° C. for subsequent cytokine analysis. Then intracellular protein transport inhibitor Brefeldin A (BFA) was added to T cells alone and to co-culture samples at a 1:2000 dilution to enable intracellular cytokine staining. Further incubation was done at 37° C. with 5% CO₂ for another 4 hrs. Subsequently, all cells in each sample were collected for cell surface marker and intracellular cytokine staining.

Additional wells of MDA-MB-468 and MCF7 MUC1 KO cells were plated on the same day of staining to evaluate cell surface Tn/STnMUC1 and MUC1 expression after 24 hr of plating. MDA-MB-468 and MCF7 MUC1 KO cells were also characterised for Tn/STnMUC1 & MUC1 expression at the time of plating.

### Cell Surface Tn/STnMUC1 and MUC1 Expression

MDA-MB-468 and MCF7 MUC1 KO cells were detached from culture flasks, then washed with DPBS twice. 1×10⁵ cells were then stained separately for cell surface Tn/STnMUC1 and MUC1 with the addition of 50 μL of a primary antibody diluted in FACS buffer and incubated for 45 minutes at room temperature in the dark. Cells were then washed twice with FACS buffer before adding 50 μL of Brilliant Violet 421™ Goat anti-Mouse secondary antibody diluted in FACS buffer and incubating for a further 45 minutes at room temperature in the dark. Cells were washed a further two times with DPBS and then resuspended in 100 μL DPBS containing Sytox Advanced viability dye diluted to 1:2000 before running on a 5-laser LSR II cytometer.

### Cytometer Set-Up & Compensation

CS&T beads were used daily to evaluate cytometer performance and inform accurate application settings for aligned acquisition of data across each timepoint. Compensation was calculated prior to the acquisition of sample data in FACSDiva using Invitrogen Ultra Comp eBeads stained with each antibody fluorochrome conjugate individually.

### Experimental Protocol(s)

### Fc Blocking and Cell Surface Staining

After collecting the cells, all samples were wash twice with DPBS before staining with 100 μL of viability dye Zombie Aqua diluted 1:2000 in DPBS and incubated for 15 minutes at room temperature in the dark. After staining CAR-T cells were washed twice with DPBS. Then Fc receptor blockage was done by resuspending cells in 40 μL of FACS buffer containing excess of irrelevant Human IgG Isotype Control diluted 1:50 (stock concentration: 5 mg/mL) with incubation of 15 minutes at room temperature in the dark. CAR-T cells were then stained with the addition of 40 μL of a 2× concentrated antibody cocktail containing CD3 and CD8 antibody-fluorochrome conjugates diluted in FACS Buffer for 30 minutes in the dark at room temperature.

### Intracellular Cytokine Staining

After cell surface staining incubation, CAR T cells were washed twice with 200 μL of FACS buffer before being fixed in 100 μL of BD Fix/Perm buffer at 4° C. in the dark for 20 minutes. CAR T cells were then washed twice with 100 μL of DPBS and permeabilised with washed in 200 μL of BD Perm/Wash buffer. CAR T cells were then stained with 50 μL of an antibody cocktail containing all intracellular cytokine (IL-2, IFNγ, TNFα, GmzB) antibody-fluorochrome conjugates diluted in BD Perm/Wash Buffer and incubated for 45 minutes in the dark at room temperature. CAR T cells were washed a further two times with 200 μL of BD Perm/Wash buffer before resuspending in 100 μL of DPBS. Samples were analysed on an LSRII cytometer.

### Supernatant Cytokine Detection Using Cytokine Bead Array (CBA) Assay

A BD™ CBA Human Th1/Th2 Cytokine Kit II and BD™ CBA Human Granzyme B Flex Set D7 were combined into one multiplex assay and used to analyse supernatant cytokine levels of IL2, IL4, IL6, IL10, IFNγ, TNFα & granzyme B. Frozen supernatants were thawed at room temperature. Assay top standards were prepared by combining lyophilised protein of each cytokine provided in each kit and reconstituting in 1 mL of cell culture media (used as assay diluent). Assay top standards were serial diluted in cell culture media to create assay standards. 50 μL of supernatants and assay standards were transferred to a 96-well, v bottom plate for assaying. A capture bead master mix was prepared containing capture beads for all 7 cytokines by combining 4 μL of each of the CBA Human Th1/Th2 Cytokine Kit II Capture Beads & 0.5 μL of the CBA Human Granzyme B Flex Set D7 Capture Beads (diluting granzyme B capture beads 1:50) for each test sample. A PE Detection Reagent master mix was also prepared by combining 25 μL of CBA Human Th1/Th2 Cytokine Kit II PE Detection Reagent & 0.5 μL of CBA Human Granzyme B Flex Set D7 PE Detection Reagent (diluting granzyme B PE detection reagent 1:50) for each test sample. 25 μL of the capture beads master mix and 25 μL of the PE detection reagent master mix were added to supernatant and assay standards at the same time. Plates were sealed and incubated with shaking (600 rpm) for 3 hrs at room temperature in the dark. Plates were washed with the addition of 100 μL of CBA Human Th1/Th2 Cytokine Kit II Wash Buffer and centrifuged at 300×g for 5 minutes. Washing was repeated before resuspended in 60 μL wash buffer before running on an LSRII cytometer.

### Supernatant Cytokine Detection Using Luminex Assay

Antigen standard set was reconstituted in a 4-fold serial dilution using the PCR 8-tube strip provided in the assay kit. The 96-well plate was attached to a Hand-Held Magnetic Plate Washer. Magnetic bead solution was first vortexed for 30 sec and 50 μL of the bead solution was added to the plate and washed once with 150 μL of Wash Buffer (1×). The plate was removed from the Hand-Held Magnetic Plate Washer and 50 μL of standards, controls or samples were added into each well, then sealed, incubated with shaking at room temperature for 120 minutes. After incubation, the 96-well plate was put back on Hand-Held Magnetic Plate Washer and washed twice with 150 μL of Wash Buffer (1×). Next, 25 μL of Detection Antibody Mixture was added, sealed and incubated for 30 minutes on a plate shaker at room temperature at 500 rpm. The plate was put back on the Hand-Held Magnetic Plate Washer and washed twice with 150 μL of Wash Buffer (1×). 50 μL of Streptavidin-PE solution was added, sealed and incubated for 30 minutes on a plate shaker at room temperature at 500 rpm. Finally, the plate was put back on the Hand-Held Magnetic Plate Washer and washed twice with 150 μL of Wash Buffer (1×), then 120 μL of Reading Buffer was added into each well, sealed, shaken at room temperature for 5 min before putting on the Luminex™ instrument ‘Bio-Plex 3D Suspension Array System’ for data acquisition.

### Data Analysis

Flow cytometry data was analysed using Flowlogic 7.2.1 software producing primary metrics and plots. Metrics were analysed in Microsoft Excel and GraphPad Prism 7. Gating strategy: CD4+ and CD8+ T cell subsets were compared in transduced (ZsGreen+) and in untransduced (ZsGreen-) T cells populations. Transduced population only data is shown for CAR T cells. Percentages and median fluorescence intensity values were exported as primary metrics. GraphPad Prism 7 was used to graph metrics representing results from 4 donors.

For CBA assay: GraphPad Prism v7 software was used to calculate standard curves using a polynomial: second order (Y=A+B*X+C*X\hat{ }2) non-linear curve fit model.

Interpolation from standard curves was used to determine concentrations of cytokine in pg/mL.

For Luminex assay: Luminex xPonent v4.2 software was used to acquire data on the machine and BioPlex Manager v6.1 was used to analyse the data including standard curve generation and the sample concentration (in pg/mL) calculation by interpolation from standard curves. Graphing was performed in GraphPad Prism for sample comparisons.

### Results

### Transduction Efficiency (TE) and CD4+/CD8+ Ratio

There are slight decreases of TE (based on % zsGreen positive cells) for all four HuCARs after 24 hr co-culturing with both tumour cell lines (MDA-MB-468 and MCF7 MUC1 KO), compared to the single culture samples (FIG. 31, A left and middle charts). And there are no obvious changes for the transduced CD4+/CD8+ ratios for all four HuCARs after 24 hr co-culturing with both tumour cell lines, compared to the single culture samples. However, there are more CD8+ cells present (˜70%) in untransduced T cell samples, whereas the majority cells in all transduced sample are CD4+ cells (FIG. 31, A right chart).

### Intracellular Cytokine Analysis

To evaluate the expression level of cytokines, intracellular staining was done at 24 hr post single culture or co-culturing T cells with either TnMUC1 positive cell line MDA-MB-468 or negative cell line MCF7-KO. The expression level was evaluated by the positive percentage of cells and degree of positivity, in terms of medium fluorescent intensity (MFI) (FIG. 31, B&C). Overall, all four huCARs show similar pattern of expression for all cytokines tested in response to TnMUC1-specific antigenic challenge with MBA-MB-468. The response in absence of tumors cells (i.e., T cell alone) from MB022 and MB025 was slightly higher than MB021 and MB024. Specifically, IL-2 shows about two-fold amount of TnMUC1 induced specific % expression in CD4+ subsets compared with CD8+ subsets. IFNγ shows about two-fold amount of TnMUC1 specific expression in CD8+ subsets compared with CD4+ subsets. TNFα shows similar trend in both CD4+ subsets and CD8+ subsets. Granzyme B is mainly expressed in all CD8+ subsets, as expected and one donor 74 showed high constitutive level of Granzyme B, even in absence of stimulation. Responses to MCF7-KO line were similar to MDA-MB-468 for MB021 and MB024, whereas responses by MB022 and MB025 were approximately two-fold higher to MCF7-KO line for all cytokines and granzyme B for both CD4+ and CD8+ T cells. Similar trends in the MEI were observed between groups although effects were more modest and variable when compared with data shown as % expression.

### Supernatant Cytokine Analysis

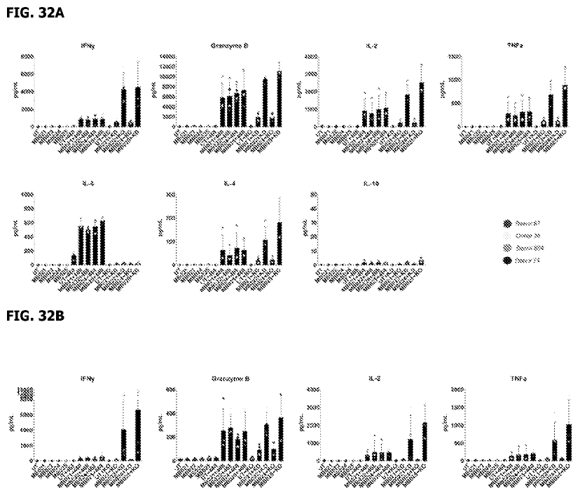

To assess the actual cytokine secretion amount that T cells secreted in the supernatant, we have used two techniques, namely Cytokine Bead Array (CBA) assay and Luminex assay (FIGS. 32A-32B). Overall, both assays showed that all four huCARs secreted cytokines in response to TnMUC1-specific antigenic challenge with MDA-MB-468 after co-culturing with this TnMUC1 positive cell line, compared with huCARs cultured in absence of tumor cells. However, MB022 and MB025 showed relatively higher response than MB021 and MB024 when co-cultured with TnMUC1 negative cell line MCF7-KO.

IL-2, IFNγ, TNFα and Granzyme B secretion levels were similar between CAR-T cells after TnMUC1 specific activation in co-culture with MDA-MB-468, which is significantly higher than UT and T cells alone samples. Noticeably, there are significantly higher secretion of IL-6 in all 4 HuCARs co-cultured with MDA-MB-468 tumour cells than any other sample conditions. Similar results were observed using both the CBA assay and Luminex assay systems.

### Characterisation of Tumor Cell Lines

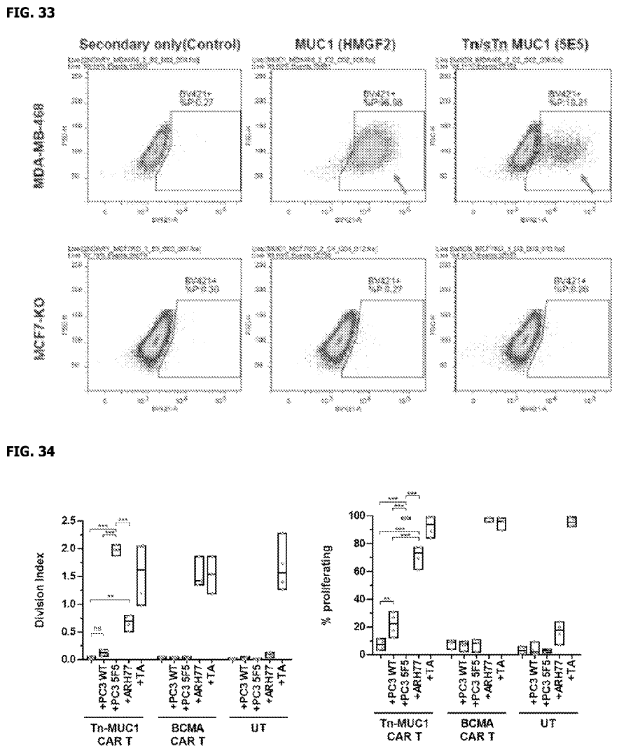

To characterise cell lines, we used for the assay, we also evaluated the TnMUC1 expression level with 5E5-CB antibody and MUC1 expression with HMFG2 antibody prior to the ICS and supernatant collection. Flow cytometry analysis showed that MDA-MB-468 cells have about 10% TnMUC1 positive cells and MCF7-KO cells are negative for both MUC1 and TnMUC1 expression (FIG. 33).

### Discussion

The success of CAR T therapies depends on a broad immune response engaging a range of effector cells and mechanisms. Highly polyfunctional T cells within CAR T-cell therapies have been reported to be significantly associated with clinical response. Recent studies showed that polyfunctional and unexhausted T cells, especially stem cell memory cells, are essential for the prolonged persistence of the therapy. We have evaluated the ability of the humanised TnMUC1 CAR T cells to secret four functional cytokines, including stimulatory cytokine—IL-2, effector cytokines—IFNγ, TNFα and cytotoxic granzyme—GrzB. We compared their secretion level in response to antigenic challenge by co-culturing either with TnMUC1 positive MDA-MB-468 or negative MCF7-KO cell lines. Single culture samples have been served as basal level comparison. We have combined cell surface, intracellular staining (ICS) and supernatant cytokine measurement together to build a complete cytokine profile for our humanised CAR products. The ICS was used to examine what our T cell products are capable of secreting—IL-2, GrzB, IFNγ and TNFα at 24 hr post co-culturing with positive and negative cell lines. Cytometric Bead Array (CBA) and Luminex assays were used to correlate what the actual secreted cytokine amount of these cytokines were in supernatant at 24 hr post co-culture with MDA-MB-468 and MCF7-KO cells. The obviously higher secretion of inflammatory cytokine IL-6 in all four HuCARs co-cultured with MDA-MB-468 tumour cells than any other sample conditions could possibly indicate its anti-tumor role, if it's secreted by T cells. But it can also possibly be secreted by MDA-MB-468 tumour cells themselves. IL-6 is a multifunctional cytokine that plays a central role in host defence due to its wide range of immune and hematopoietic activities and its potent ability to induce the acute phase response. Accumulating evidence establishes IL-6 as a key player in the activation, proliferation and survival of lymphocytes during active immune responses.

A general summary of the T-cell polyfunctionality assay for the four tested HuCARs is shown below in Table 26. This table showed that the overall expression levels of four examined cytokines, after considering what the cells were capable of secreting intracellularly vs the actual secretion in the supernatant. The general consistent trend is the more the cells are capable of secreting, the more actual secretion is observed in the supernatant. We observed that MB022 and MB025 seems to have very high non-specificity towards negative cell line MCF7-KO, which has no expression of MUC1 and TnMUC1. In contrast, MB021 and MB024 demonstrated relatively higher specificity to TnMUC1, as evident from significantly lower levels of secreted cytokines when CAR-T cells were co-cultured with MUC1 negative cell line—MCF7-KO.

TABLE 26
Summary of T-cell polyfunctionality assay
for HuCARs MB021, MB022, MB024, & MB025

| Corresponding | | | | | |
|---|---|---|---|---|---|
| LNGFR- | | | | | |
| version huCARS | | TNFα | IFNγ | IL-2 | GrzB |
| | TnMUC1-specific | | | | |
| | response | | | | |
| MB037 | MB021 + MDA- | + | ++ | ++ | +++ |
| | 468 | | | | |
| MB039 | MB022 + MDA- | + | + | + | +++ |
| | 468 | | | | |
| MB040 | MB024 + MDA- | + | + | ++ | +++ |
| | 468 | | | | |
| MB041 | MB025 + MDA- | +/− | +/− | +/− | +++ |
| | 468 | | | | |
| | Non-specific | | | | |
| | response | | | | |
| MB037 | MB021 + MCF7 | + | +/− | ++ | + |
| MB039 | MB022 + MCF7 | +++ | + | +++ | ++++ |
| MB040 | MB024 + MCF7 | + | +/− | ++ | + |
| MB041 | MB025 + MCF7 | ++ | ++ | ++ | ++++ |

Understanding the complex polyfunctionality of key cytokines, together with analysing cell surface phenotypic biomarkers will help the identification of their relations with patient response in CAR T therapies. For example, although IL-2 can support CAR T cells in vivo and has been tested preclinically and in many clinical trials, it has been reported that it may actually preferentially activate and induce proliferation of Tregs. These data highlight the complex role of IL-2 in CAR T therapies. Other studies that correlate polyfunctionality with T cell subpopulations showed that polyfunctional CD4+ T cells (IFNγ+/IL2+/TNFα+) were significantly associated with recurrence-free survival of patients with bladder cancer. Furthermore, the highly polyfunctional T cells shall correlate with their proliferative potential and apoptosis rate. As highly polyfunctional memory T cells, for instance, are reported to possibly co-produce many cytokines (such as IFNγ, TNFα and IL-2) so that they can become cytolytic and proliferate vigorously. These cells also have considerable survival capacity and are maintained long term without antigen.

Further application of using measurements like polyfunctional strength index will consolidate high-dimensional, single-cell protein secretion data into a single metric that represents the overall activity of examined samples. These measurements can help to reveal possible correlations of CAR T cell subsets and mechanisms of therapies that eventually enhance lead choice.

### Example 15: In Vitro Proliferation of Tn-MUC1 CAR-T in Response to Tumor Cell Lines

Clinical response to CAR T therapy can be in part attributed to robust in vivo expansion and persistence of engineered T cells after infusion. Because of this, proliferation can be used to measure potential therapeutic efficacy of CAR T cells in vitro. The objective of this study was to determine the proliferative ability of TnMUC1 CAR-T cells to tumour cell lines expressing Tn/STn-MUC1 antigen (“Tn-MUC1”).

### Methods

### Experimental Preparations

### CAR T Cell Generation and Preparation

The CAR-T cells were generated as described previously.

### Cell Surface Tn-MUC1, MUC1 and BCMA Expression by Flow Cytometry

Cell lines PC3 (COSMC KO) 5F5 clone (PC3 5F5), PC3 WT and ARH77 clone 10B5 (ARH77 BCMA) were detached from culture flasks with TrypLE™ Express and washed in FACS buffer (DPBS+2% FBS+0.5% sodium Azide). Cells were then centrifuged at 300×g for 5 minutes and resuspended to 1×10⁶ cells/mL in FACS buffer in preparation for staining. 1×10⁵ cells were stained separately for Tn-MUC1, MUC1(HMFG2) and BCMA. Tn-MUC1, MUC1 indirect staining: Staining was carried out in 50 μL of a primary antibody diluted in FACS buffer and incubated for 45 minutes at 4° C. in the dark. Cells were then washed twice with FACS buffer before adding 50 μL of Brilliant Violet 421™ Goat anti-mouse secondary antibody diluted in FACS buffer and incubated for a further 45 minutes at 4° C. in the dark. Cells were washed a further two times with DPBS and then resuspended in 100 μL DPBS containing Sytox orange viability dye diluted to 1:2000. BCMA direct staining: Staining was carried out in 50 μL of antibody diluted in FACS buffer and incubated for 45 minutes at 4° C. in the dark. Cells were then washed twice with FACS buffer and then resuspended in 100 μL DPBS containing Sytox Orange viability dye diluted to 1:2000. Samples were ran on a CytoFLEX cytometer. CytoFLEX QC beads were used to evaluate cytometer performance before acquiring data.

### Experimental Protocols

### VPD450 Proliferation Dye Labelling of CAR T/UT T Cells

T cells were harvested in 15 mL falcons after 48 hr culture in low IL-2. T cells were then washed twice in 10 mL Dulbecco's phosphate-buffered saline buffer (DPBS), counted and resuspended to a concentration of 10×10⁶ cells/mL in DPBS. A 4 μM (2×) concentration of VPD450 proliferation dye diluted in DPBS was added to T cells to a volume double of the initial cell suspension (2 μM final reaction concentration of VPD450). Unlabelled T cell controls were incubated with DPBS only. T cells were Incubated with VPD450 for 15 minutes at 37° C., protected from light. Following incubation, T cells were washed once in 10 mL DPBS and then in 10 mL room temperature co-culture media. T cells were incubated for 15 minutes in co-culture media to allow acetate hydrolysis of the VPD450 dye before being plated in co-culture with target cells.

### Co-Culture Set Up

Co-cultures were performed in flat bottom 48-well cell culture treated plates at a 1:1 C:T ratio. C:T ratios were calculated based on percentage transduction efficiency in which transduction efficiency was normalised across different donors and constructs. 3.33×10⁵ tumour cells were plated per well from three Tn-MUC1 positive cancer cell lines with high (PC3 5F5) or low (PC3 WT and ARH77 BCMA) levels of antigen expression 24 hrs prior to CAR T/UT T cell addition. MB053 TnMUC1 CAR T cells, BCMA CAR T cells or UT T cells were added to tumour cell lines at 0 hrs and co-cultured for 96 hrs in a humidified incubator at 37° C. with 5% CO₂. For non-specific CD3/CD28 stimulated T cell controls, T cell TransACT beads were added to CAR T/UT T cells at a 1:50 final assay dilution.

### T Cell Phenotyping—Flow Cytometry

CAR T/UT T cells were harvested from co-cultures into 15 mL Falcon tubes and the cell density acquired using a cell counter. 2×105 cells per condition were plated into a 96-well V bottom plate and centrifuged at 300×g for 5 minutes at room temperature, the supernatant removed, and cells washed in DPBS. Following a repeated wash, Fc receptors were blocked by adding Human Fc receptor blockage was carried out in 40 μL Trustain FcX diluted 1:50 in FACS Buffer for 15 minutes at room temperature. Cells were subsequently stained with the addition of 40 μL of a 2× concentration of anti-fab(2) diluted in FACS Buffer. Cells were incubated with anti-fab(2) for 30 minutes in the dark at room temperature. Cells were then washed twice with DPBS and were stained with 40 μL of an antibody cocktail containing CD3, CD8 & BCMA transduction markers as well as a streptavidin-PE secondary for anti-fab(2) detection. Cells were incubated for 30 minutes in the dark at room temperature. Cells were washed a further two times with DPBS and resuspended in 100 μL of DPBS containing Sytox green diluted 1:2000. T cells were incubated with Sytox green for 15 minutes at RT in the dark before being read on a BD X-20 flow cytometer. Cytometer setup and tracking (CS&T) beads were used to evaluate cytometer performance. Compensation was calculated prior to the acquisition of sample data in FACS Diva using Invitrogen ultra Comp eBeads stained with each antibody fluorochrome conjugate. Flow cytometry data was analysed using Flowlogic 7.2.1 software.

### Results and Discussion

### Tn-MUC1 CAR T Cell Proliferation in Response to Antigenic Stimulation

MB053 CAR-T cells consistently and robustly proliferated when co-cultured with Tn-MUC1 expressing cancer cell lines and the level of MB053 CAR-T cell proliferation was defined by the extent of Tn-MUC1 expression on cancer cells.

A high expressing Tn-MUC1 expressing cell line (PC3 5F5) was generated from a low Tn-MUC1 expressing cell line (PC3 WT) by knock-out of COSMC. COSMC is molecular chaperon for T synthase—a key enzyme involved into Tn-antigen elongation in normal cells. As a result, PC3 5F5 cells lacking T synthase function express high level of Tn-MUC1 on the cell surface. PC3 5F5, PC3 WT, and ARH77 BCMA cancer cell lines all expressed Tn-MUC1 on the cell surface to various degrees. 96.0% of PC3 5F5 cells were positive for Tn-MUC1. In comparison, only 2.97% and 1.89% of ARH77 BCMA and PC3 WT cells were Tn-MUC1 positive respectively. The relative amount of Tn-MUC1 expression was also significantly higher in PC3 5F5 compared to PC3 WT. BCMA was expressed in 98.3% of ARH77 BCMA cells whereas PC3 5F5 and PC3 WT cells were negative for BCMA (comparable to background). All three cell lines expressed MUC1. PC3 WT and PC3 5F5 expressed similar levels with 81.8% and 94.5% of cells positive for MUC1 respectively. ARH77 BCMA cells expressed lower levels of MUC1 at 65.9%.

On average 92% of MB053 CAR T cells proliferated in response to challenge with Tn-MUC1 positive cell line PC3 5F5 (FIG. 34). In comparison, control BCMA CAR T cells and UT T cells did not proliferate in co-culture with PC3 5F5 (7% and 2% proliferating respectively) and were comparable to when cultured alone. MB053 CAR-T cell proliferation in response to Tn-MUC1 antigen was also shown to be equivalent to TCR-mediated stimulation with TransACT (˜95%). The proliferative response to CD3/C28-mediated stimulation was equivalent across MB053 CAR-T cells, BCMA CAR T and UT T cells.

To further understand the extent of MB053 CAR-T cell proliferation, division index (DI), proliferation index (PI) and fold expansion were calculated. DI calculates average number of divisions within the whole population of dividing and non-dividing cells. MB053 CAR T cells underwent 2 cell divisions on average when challenged with PC3 5F5 compared to 0.04 and 0.02 divisions underwent by BCMA CAR T and UT T cells respectively when challenged with the same cell line. DI of MB053 CAR-T cells in response to PC3 5F5 was also higher than DI of BCMA CAR T challenged with BCMA-positive ARH77 BCMA cell line or CD3/CD28 stimulated T cells (DI 1.5 and 1.6 respectively). DI was similarly reflected in the overall expansion in which MB053 CAR-T cells showed a 4.8-fold expansion in response to PC3 5F5 compared to a 4.2-fold expansion when stimulated with TransACT. No significant proliferative difference was observed between CD4 and CD8 MB053 CAR T cells in response to Tn-MUC1 in which CD4/CD8 ratios were largely unchanged compared to CAR T culture alone or stimulated with TransACT.

MB053 CAR-T cells were also shown to partially proliferate in co-culture with ARH77 BCMA cells and PC3 WT cells which both express low levels of Tn-MUC1 (2.97% and 1.89% respectively). Compared to 92% MB053 CAR-T cell proliferation to challenge with PC3 5F5, only 20% proliferated in response to PC3 WT whereas 80% proliferated to challenge with ARH77 BCMA cells. However, the small proportion of MB053 CAR T proliferating in response to PC3 WT cells did not invoke any significant increase in either the DI (p=0.101) or overall expansion (p=0.417) when compared to MB053 CAR-T cells cultured alone. As the DI measures proliferation within the whole system (in responding and non-responding CAR T cells), the low DI reflects the lesser extent of proliferation observed in responding CAR T cells (evident by the proliferation index which considers only responding cells) as well as by the low proportion of CAR T cells able to respond.

Together, this indicates an overall weak proliferative response of MB053 CAR-T cells to PC3 WT. However, challenge with ARH77 BCMA cells induced a two-fold expansion of MB053 CAR-T cells with a significant increase in the DI albeit lower compared with PC3 5F5 challenge (PC3 5F5 DI=2 vs. ARH77 BCMA DI=0.6). Interestingly, ARH77 BCMA cells were able to induce UT T cell proliferation which was not observed with either PC3 WT or PC3 5F5 co-cultures (FIG. 34). Taking into account the background effects of ARH77 BCMA cells on T cell proliferation, MB053 CAR-T cell proliferation to ARH77 BCMA challenge was still significantly greater than with PC3 WT (FIG. 34).

Proliferation of MB053 CAR-T cells in response to ARH77 BCMA cells was vastly different to that with PC3 WT despite comparable Tn-MUC1 expression levels (2.97% vs 1.89%, respectively). The difference in MB053 CAR-T cell proliferation observed in response to PC3 WT and ARH77 BCMA cells is likely related to cell line characteristics which influence the interplay between tumour and T cell, independent of Tn-MUC1 antigen.

Taken together, MB053 CAR-T cells showed antigen-dependent proliferation in response to challenge with Tn-MUC1 antigen which was equivalent to antigen-independent TCR mediated stimulation with TransACT.

### CAR Expression and CD4/CD8 Ratios in Antigen Challenged TnMUC1 CAR-T Cells

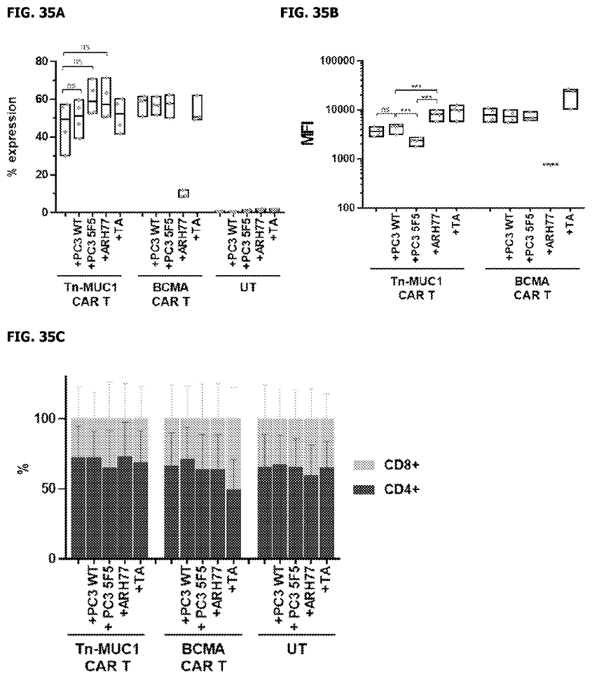

In addition to evaluating MB053 CAR-T cell proliferation, CAR expression on T cells was monitored before and after co-culture with target cell lines (FIGS. 35A-35C).

CAR expression on MB053 CAR T cells was influenced by the strength of antigen stimulation. Strong antigenic challenge induced a downregulation of CAR whereas intermediate to low antigenic challenge upregulated CAR expression on the T cell surface of MB053 CAR T cells after 4 days of stimulation. No significant difference was observed in the frequency of CAR positive cells between MB053 CAR-T cells challenged with either PC3 WT or stimulated with TransACT as compared to CAR level when MB053 CAR-T cells were cultured alone.

Frequency of BCMA CAR positive cells significantly dropped from ˜60% to ˜15% when anti-BCMA CAR T cells were challenged with BCMA positive ARH77 BCMA cells. However, frequency of BCMA CAR positive cells was not affected in co-culture with BCMA negative PC3 WT or PC3 5F5 or in response to CD3/CD28 stimulation.

Overall, CAR expression on MB053 CAR T cells was shown to be influenced by different levels of antigenic stimulation—with strong antigen stimulation resulting in loss of CAR expression whereas intermediate-low stimulation increasing CAR expression. Activation of MB053 CAR T cells via endogenous TCR using TransACT generally increased CAR expression.

CD4/CD8 ratios of MB053 CAR T cells were also evaluated. No significant change was observed in the CD4/CD8 ratio with either CAR-mediated or endogenous TCR-mediated activation of MB053 CAR T cells after 4 days compared to MB053 CAR T cells cultured alone (FIG. 35C).

Similarly, BCMA CAR was also shown to be downregulated with strong antigen stimulation. Transient CAR downregulation upon antigen stimulation is widely reported in the literature. It is considered likely that the downregulation of CAR is due to internalisation in response to CAR stimulation of which is greater in the presence of high antigen expressing PC3 5F5 cells compared to ARH77 BCMA or PC3 WT cells. However, the increase in CAR expression in response to ARH77 BCMA or PC3 WT is likely due to the combination of lesser CAR internalisation due to lower antigen, whilst increasing CAR expression due to the general activation of CAR T cells. Similarly, CD3/CD28 stimulation increase of CAR expression is also likely due to increased activity in CAR T cells which indirectly increased CAR synthesis and/or surface expression.

Taken together, dynamic regulation of CAR expression upon antigen stimulation was similar to previously published findings with other CAR T cells. MB053 CAR-T cells demonstrated a strong ability to proliferate in a Tn-MUC1 dependent manner. Robust proliferation is a hallmark of successful CAR T cell therapy and as such demonstrates efficacy of MB053 CAR-T cells as a CAR T cell therapy.

### Example 16: CAR-T Binding to Cell Lines Overexpressing DCC Netrin 1 Receptor

This study was performed to assess the binding of TnMUC1 CAR-T cell populations to DCC netrin 1 receptor, in order to validate the results in Example 6 above in which MB037 CAR-T cells (transduced with LNGFR tagged lentiviral vector) were found to bind to the DCC netrin 1 receptor with medium intensity. In this study, CAR-T cells transduced with the tagless lentiviral vectors (i.e., lentiviral vector lacking the LNGFR tag) were co-cultured with cell lines overexpressing DCC. In particular, the binding of MB051 CAR T cells (the LNGFR tagless variant of MB037) and MB053 CAR-T cells in co-culture with cell lines overexpressing DCC were evaluated.

### Methods

### Experimental Preparations

### Transduction and Expansion of T Cells

T cells were transduced and expanded essentially as previously described. Briefly, T cells were harvested from PBMCs and transduced with D4 Tn-MUC1 lentiviral vector (to generate MB051 CAR-T cells) or D16 Tn-MUC1 lentiviral vector (to generate MB053 CAR-T cells) at an MOI of 3. T cells were spinnoculated at 800×g for 2 hours at 32° C. prior to incubation in a humidified incubator at 37° C. with 5% CO₂ for two days. Two days post-transduction, T cells were expanded in TEXMACs media supplemented with 100 IU/mL of IL-2. After three days, 100 IU/mL of IL-2 was added to each well. After a further two days, media was changed by removing 6.5 mL of TEXMACs media and replacing with 6.5 mL of fresh TEXMACs media, supplementing with 100 IU/mL of IL-2. After a further two days, 100 IU/mL of IL-2 was added to each well. T cells were incubated for a further three days before being harvested, counted and frozen for use in co-culture assay. At day 12 post-transduction, T cell populations were harvested and samples of T cells were removed for analysis of transduction efficiency by fluorescence activated cell sorting (FACS) using anti-Fab′(2)-Biotin antibody (Ab) (Jackson ImmunoResearch, #115-066-072) and PE-Streptavidin secondary Ab (Miltenyi Biotec, #130-106-789) on a MACSQuant Analyser 10 Flow Cytometer.

Transfection of Cell Lines with DCC Plasmid

HEK293Tsa cells were harvested from culture vessels, centrifuged, resuspended in HEK293Tsa media and counted on the ViCell XR. Cells were resuspended at 4E6 cells/mL and 2E6 cells were plated into a 96 well deep well plate. Cells were transfected with 8 μg plasmid using the transfection reagent 293Fectin. Cells were incubated at 37° C. and 5% CO₂ for 72 hours. Subsequent to transfection, cell lines were analysed via flow cytometry to determine the expression of DCC netrin 1 receptor and ZsGreen, which was used as a marker of transfection. Samples of cell solution were plated into V bottom 96 well polypropylene plates, washed with FACs buffer and blocked with 50 μL of 40 μg/mL human IgG solution for 5 minutes at RT. Cells were washed with FACs buffer and cell pellets were stained with 50 μL of 10 μg/mL of Mouse anti-DCC primary Ab (BD Pharmingen, #554223) or 50 μL of 10 μg/mL Mouse IgG1 isotype control Ab for 30 minutes at room temperature (RT). Cells were washed twice with FACs buffer and stained with 50 μL of 5 μg/mL of APC anti-mouse IgG1 Secondary Ab (BioLegend, #406610) for 30 minutes at RT. Cells were washed twice with FACS buffer and stained with 100 μL of 1 μg/mL DAPI solution, prior to analysis on either the CytoFLEX S flow cytometer or MACSQuant Analyser 10 Flow Cytometer.

Detection of DCC Expression—qPCR

SYBR Green-based real time qPCR was used for relative quantification of the DCC mRNA expression. RNA was extracted with the RNeasy Plus Mini kit (Qiagen). The total RNA was then reverse transcribed into cDNA using the Superscript™ IV First-Strand Synthesis System according to manufacturer's guidelines. Transgene-derived DCC was quantified and normalised against endogenous ACTB which was used as reference control.

### Detection of DCC Expression—Western Blot

Cell pellets were lysed on ice in 50 μL of RIPA buffer with protease inhibitors, with three 5 minute incubations and 30 seconds of vortexing between each incubation. The cell lysates were centrifuged for 15 minutes at 13,000×g for 4° C. before lysates were transferred to fresh tubes, ensuring no cell debris was transferred. A Pierce BCA assay was performed according to the manufacturer's protocol for total protein quantification. NuPAGE SDS Electrophoresis was performed according to the manufacturer's protocol. Membrane, filter paper and blotting pads were soaked in transfer buffer for 30 minutes and SDS gels were incubated in transfer buffer for 3 minutes. A transfer sandwich was set up in an Xcell II transfer module and the gel was transferred for 1 hour at 30 V and 170 mA. The membranes were washed in PBS, blocked in Odyssey blocking buffer, and then stained with primary antibody followed by staining with secondary antibody before imaged using a Licor Odyssey imager and Lite Studio software.

### Analysis of Tn-MUC1 and MUC1 Expression on HEK Cells

Samples of cell solutions were plated into V bottom polypropylene 96 well plates and washed with FACs buffer and blocked with 50 μL of 40 μg/mL human IgG solution for 5 minutes at RT. Cells were washed and cell pellets were stained with 50 μL of 1 μg/mL 5E5 Ab, 50 μL of 2 μg/mL HMFG2 Ab (Absolute Antibody, Ab.00712-1.1) or 50 μL of 1 μg/mL Mouse IgG1 Isotype control Ab for 45 minutes at 4° C. Cells were washed twice with FACs buffer and stained with 50 μL of 2 μg/mL BV421 goat anti-mouse IgG secondary antibody (BioLegend, 405317) for 45 minutes at 4° C. Cells were washed twice with FACs buffer and stained with 100 μL of Sytox AADvanced (1 in 2000 dilutionand incubated for 10 minutes at RT prior to analysis on the CytoFLEX S flow cytometer.

### Co-Culture of CAR-T Cells and Cell Lines

48 hours post-transfection, HEK cells (untransfected, ZsGreen transfected and DCC-ZsGreen transfected), WT Jurkat cells and COSMC Jurkat cells were harvested, counted, washed with PBS and then resuspended to a cell density of 2E6 cells/mL in RPMI media. CAR T cells were thawed, washed and resuspended in TEXMACs media to a density of 1E6 cells/mL. IL-2 (final concentration 100 IU/mL) was added to the cell solution. Cells were plated into flat bottom 24 well cell culture plates adding 1E6 cells per well and were incubated in a humified incubator at 37° C. with 5% CO₂ for 24 hours. After a recovery period of 24 hours, T cells were harvested from 24 well plates, re-counted and 6E6 cells from each cell population were transferred into 15 mL tubes. Cells were washed with PBS twice and resuspended in RPMI media to achieve a cell density of 2E6 cells/mL.

100 μL (2E5 cells per well) of cell lines and 100 μL (2E5 cells per well) of T cells were plated into 96 well flat bottom plates, separating the four donors across four plates. The conditions set up within the co-culture experiment were: T cells alone; untransfected HEK cells+T cells; ZsGreen transfected HEK cells+T cells; DCC transfected HEK cells+T cells; COSMC Jurkat cells+T cells; WT Jurkat cells+T cells; and all cell lines cultured alone. Plates were incubated within a humidified chamber at 37° C. with 5% CO₂ for 48 hours.

Samples of CAR T cells were analysed for CAR expression, DCC & ZsGreen expression by flow cytometry, Tn-MUC1 and MUC1 expression by flow cytometry, DCC gene expression by qPCR, and DCC protein expression by Western Blot. After 48 hours, plates were removed from the incubator, centrifuged at 300×g for 5 minutes and 100 μL of supernatant was removed from each well and transferred into 96 well V bottom plates. The plates were sealed and supernatants frozen at −80° C. until MSD analysis was performed.

### MSD Analysis of Supernatants

Stored supernatants were thawed at room temperature. Samples and calibrators were prepared as per manufacturer's instructions and 25 μl of each sample were added to the MSD plate. Plates were sealed and incubated at room temperature with shaking. Plates were washed 3× with PBS+0.5% Tween (Sodexo) using the plate washer. Detection antibody was diluted in diluent 100. Following the addition of 25 μl of detection antibody, the plates were sealed and incubated at room temperature with shaking for 1.5 hours. Plates were washed as before. 150 μl of 2× read buffer was added to each well before reading on the MSD Sector 600 Imager.

### Results and Discussion

### Tn-MUC1 and MUC1 Expression Analysis

Cell lines, including wild-type (WT) Jurkat cells, COSMC Jurkat cells, WT HEK cells and A673 cells were analysed for the expression of Tn-MUC1 and MUC1 via flow cytometry to determine background levels of Tn-MUC1 expression which may result in activation of Tn-MUC1 CAR T cells independent of DCC binding. WT Jurkat cells were 100% positive for Tn-MUC1, which was expected as WT Jurkat cells are the positive control for Tn-MUC1. There was 13% expression of Tn-MUC1 within the COSMC Jurkat cell population, however the median fluorescence intensity (MFI) of BV421 within the single cell population was low-demonstrating that this is likely just very low levels of background staining within the Tn-MUC1 negative COSMC Jurkat cell population. There was also a low frequency of Tn-MUC1 expressing cells within A673 and HEK cell populations, with MFIs four time higher than that of the COSMC Jurkat cells. This could be background staining or may indicate a very low level of Tn-MUC1 expression on these cell lines.

Similarly, the low MFIs and low frequency of Tn-MUC1 expressing A673 and HEK cells detected were determined to be background staining. Due to the results of this staining analysis, it was determined that there was not a risk of increased Tn-MUC1 expression within DCC-ZsGreen transfected HEK cell populations.

### Transfection of DCC Plasmid for Co-Culture

Initial work performed was to optimise the transfection of cell lines with a DCC plasmid to enable the setup of co-culture assays. Several transfection methods were tried, including nucleofection and use of transfection reagents including Lipofectamine 3000, PEIpro and 293Fectin. The final transfection method used was the use of 293Fectin transfection reagent and 8 μg of plasmid. This resulted in a transfection efficiency in HEK cells of 34%, based on the detection of ZsGreen expression. Although this is a low frequency of ZsGreen expression, both the qPCR and Western blot results demonstrated a high expression of both the DCC gene and the DCC protein. Since the anti-DCC Ab not binding specifically to DCC positive cell lines, it was not possible to demonstrate the expression of DCC protein on the surface of the transfected HEK cells. However, surface expression was presumed based on qPCR, Western and MSD results.

In particular, qPCR analysis of the transfected HEK cells demonstrated high expression of the DCC gene within the DCC-ZsGreen transfected HEK cells at day 3 post-transfection compared to the housekeeping gene ACTB. The expression of the DCC gene decreased by day 5 post transfection, however expression was still higher than the expression of the DCC gene within the endogenously expressing DCC positive cell line A673. Western analysis demonstrated that the DCC protein was expressed within the DCC-ZsGreen transfected HEK cells at both day 3 and day 5 post-transfection, with a band running within the molecular weight range predicted by Becton Dickinson (168-175 kDa).

### Co-Culture and MSD

MB051 CAR-T cells and MB053 CAR-T cells, which differ by a single amino acid, were used in the co-culture assay with untransduced T cells used as the control to reduce the risk of unknown interactions between irrelevant CARs and uncharacterised cell lines, which may have led to unexpected activation of irrelevant CAR T cell populations. The results of the co-culture experiment demonstrated a large production of IFN-γ by MB051 and MB053 CAR T cells co-cultured with DCC expressing HEK cells, with levels over two times higher than the amount of IFN-γ production when co-cultured with wild type Jurkat cells, which are the Tn-MUC1 positive control. For all donors, there was a higher production of IFN-γ produced when MB051 CAR T cells were co-cultured with DCC expressing HEK cells which potentially demonstrates a stronger interaction between DCC and the MB051 CAR T cells which were originally screened in Example 6 above. MB053 CAR T cells co-cultured with DCC expressing T cells produced almost two times the amount of IFN-γ as the MB053 CAR T cells co-cultured with wild type Jurkat cells, however MB051 CAR T cells produced between 4-5 times more IFN-γ when co-cultured with DCC expressing HEK cells than wild type Jurkat cells.

As shown in FIG. 36, there was background levels of IFN-γ production when untransduced T cells were co-cultured with the cell lines. An increase in the background level of IFN-γ production was observed when MB051 and MB053 CAR T cells were cultured with untransfected and ZsGreen transfected HEK cells. The highest peak in IFN-γ production was observed when MB051 and MB053 CAR T cells were co-cultured with DCC transfected HEK cells, with a 5473 fold increase and 3239 fold increase in IFN-γ production above untransduced T cells for MB051 and MB053 CAR T cells respectively. The fold change in IFN-γ production was lower when MB051 and MB053 CAR T cells were co-cultured with the Tn-MUC1 positive control WT Jurkat cells, with a 1937 fold increase and 1280 fold increase in IFN-γ production above untransduced T cells for MB051 and MB053 CAR T cells respectively.

There was some increase in the background level of IFN-γ when both MB051 and MB053 CAR T cells were co-cultured with untransfected HEK cells and ZsGreen transfected HEK cells, which could indicate a low level of Tn-MUC1 expression on the HEK cell lines. Donor PR19X128768 and PR19W128773 also had an increase in the background production of IFN-γ within the T cell alone populations, which may indicate low level basal activation within these donors.

Overall, the results validated the results of Example 6 above, confirming the binding of MB051 CAR T cells to DCC. Further studies are being conducted to investigate binding of Tn-MUC1 CAR T cells at physiological levels of DCC, as these experiments showed DCC at overexpressed levels.

### Example 17: In Vivo Efficacy of Tn-MUC1 CAR-T in CDX NSG Mouse Model

The objective of this study was to assess the ability of Tn-MUC1 CAR-T cells (MB053) to induce targeted killing of the Tn-MUC1 expressing prostate cancer cell line PC3 5F5 in vivo. Tn-MUC1 positive cell line (PC3 5F5) was used as cell derived xenograft (CDX) which was implanted into NSG (NOD scid gamma mouse) mouse model. BCMA CAR-T cells were used as negative control.

### Methods

### Tumor Cell Inoculation and T Cell Dosing

PC3 5F5 prostate cell line was used to establish tumour xenograft model by injecting mice subcutaneously with 2×10⁶ tumour cells. Animals were briefly anaesthetised in a chamber by isoflurane-oxygen mix and moved to face cone. The right flank was shaved then wiped with alcohol wipe. Cells were resuspended in PBS and then mixed well with Matrigel on ice (1:1 PBS/cells:Matrigel). A total volume of 100 uL of Matrigel and PBS solution with cells were injected s/c per mouse.

When tumours were palpable (˜100 mm³), CAR T cells were dosed intravenously via tail vein injection at a dose of 1×10⁷ cells per mouse. 17 days post tumour engraftment, when the mean tumour volume measured by caliper reached approximately 100 mm³, mice were inoculated intravenously via tail vein injection at a dose of 1×10⁷ cells per mouse with MB053 Tn-MUC1 CAR-T cells, PBS or BCMA CAR T cells (CAR T of non-relevant specificity).

### Tumour Measurements

Tumour size in all mice was measured by caliper measurements and recorded three times a week to be followed by body weight recording twice a week. Tumour volume was calculated with Excel as indicated below:

Tumour volume=Tumour length *(Tumour Width\hat{ }2)*0.5

Mice were culled and tissues harvested at individual end points due to end point criteria such as tumour volume. For PBS control and MB053 CAR-T cell groups, tumours and essential organs (kidney, heart, spleen, brain and lungs) were collected and fixed with 10% buffered formalin saline (BFS) for up to 48 hours for histopathological examination. No tumours or organs were collected from the BCMA group of mice.

### Blood Collection

Blood samples from all mice on study were collected twice—6 days post tumour inoculation (used as baseline) and 7 days post CAR T cell dosing to assess the level of cytokines in blood released by CAR T cells.

### Serum Cytokine Assay MSD

To assess cytokine levels secreted by MB053 TnMUC1 CAR T cells and BCMA CAR T cells, blood samples (˜100 μl) were collected twice from all mice. First collection was carried out on day 7 prior to CAR T cells dosing and represented baseline (Day 0). Second blood collection was carried out 7 days post CAR T cell dosing (Day 7). The collected whole blood was allowed to clot for a minimum of 30 minutes at room temperature (RT). Once clotted the blood was centrifuged at 12500 rpm for 3 minutes to obtain serum which was frozen at −80° C. until used in the assay. All mouse serum samples collected were thawed in RT and analysed according to the manufacturer's instructions using detection antibodies against IL-2, IFN-γ, and TNF-alpha (detection antibody mix).

Plates were read on the MSD Sector 600 Imager and data analysis was performed using the brms package in R version 3.6.1. The quantification of serum cytokine concentration changes was analysed using MSD extrapolated values. This quantification was repeated in two technical replicates—i.e. two separate plates. Many observations of the control groups were so low that they were below the limit of quantification (<LLoQ) for a given mouse/time/cytokine concentration.

### Results and Discussion

### Impact of MB053 TnMUC1 CAR-T Cells on Tumour Growth in CDX Tumour-Bearing Mice

Transduction efficiency was measured for MB053 TnMUC1 CAR T cells and BCMA CAR T cells one day prior to dosing using flow cytometry with anti-Fab detection reagent and BCMA Ab, respectively. In addition, basic T cell phenotype was carried out, where CD3+/CD4+/CD8+ populations were measured. Both CAR T cell samples showed high cell viability (98-99%) and transduction efficiencies were determined as 45.92% for MB053 CAR-T cells and 74.22% for BCMA CAR T cells. Transduction efficiency was normalised to 50% with UT cells between MB053 CAR-T cells and BCMA CAR T cells.

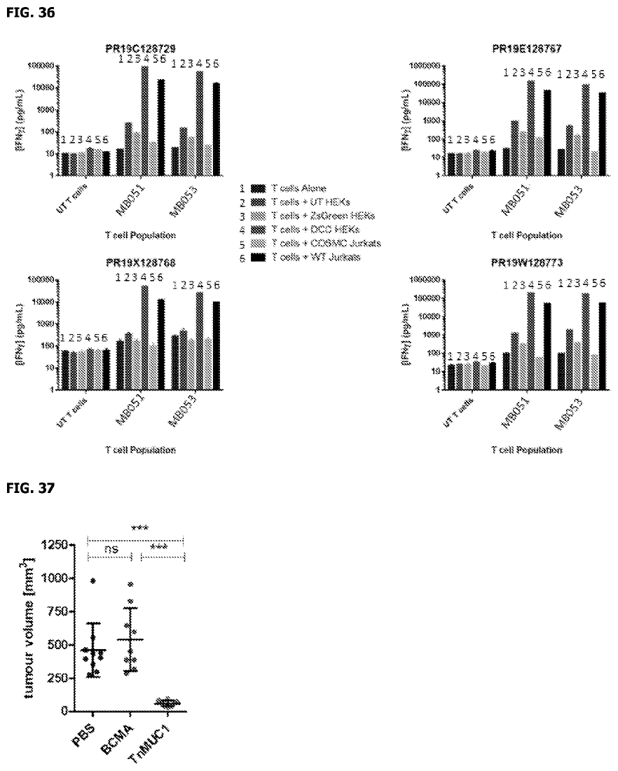

MB053 CAR-T cells had a potent anti-tumor effect, as was evident from drastic tumour volume reduction leading to tumour clearance in tumour-bearing CDX mice treated with MB053 CAR-T cells. In contrast, tumour volume reduction or tumour clearance was not observed in CDX tumour-bearing mice treated with PBS or BCMA CAR T cells. In fact, CDX tumour-bearing mice treated with MB053 CAR-T cells showed a statistically significant decrease in tumour volume even at an earlier timepoint (study day 31), before the study endpoint (FIG. 37). Histopathological examination confirmed that tumours were present only in CDX tumour-bearing mice dosed with PBS or BCMA CAR T cells. By contrast, in tumour-bearing CDX mice treated with MB053 CAR-T cells, the tumour site comprised only variable admixtures of collagen, adipose tissue and skeletal muscle but no tumour cells at all. Of note, no difference in tumour volume was seen when CDX tumour-bearing mice dosed with BCMA CAR T were compared with PBS control group.

Overall, administration of MB053 CAR T cells in CDX tumour-bearing mice in vivo resulted in tumour shrinkage. Injection of MB053 CAR T cells into tumour bearing mice resulted in fast tumour size reduction leading to complete tumour clearance. In contrast, injection of PBS or control BCMA CAR T (CAR of non-relevant specificity) into tumour bearing mice didn't result in tumour volume reduction. MB053 CAR T cells controlled the tumour growth, as seen by caliper measurements. Interestingly, even prior to study endpoint (study day 30), there was a significant reduction in tumour volume in tumour-bearing CDX mice dosed with MB053 CAR T cells compared to tumour-bearing CDX mice dosed with BCMA CAR T cells or PBS control group. Moreover, histopathological examination confirmed the absence of tumour cells in the tumour site in tumour-bearing CDX mice treated with MB053 CAR T cells.

### Serum Cytokine Release to Assess T Cell Activation

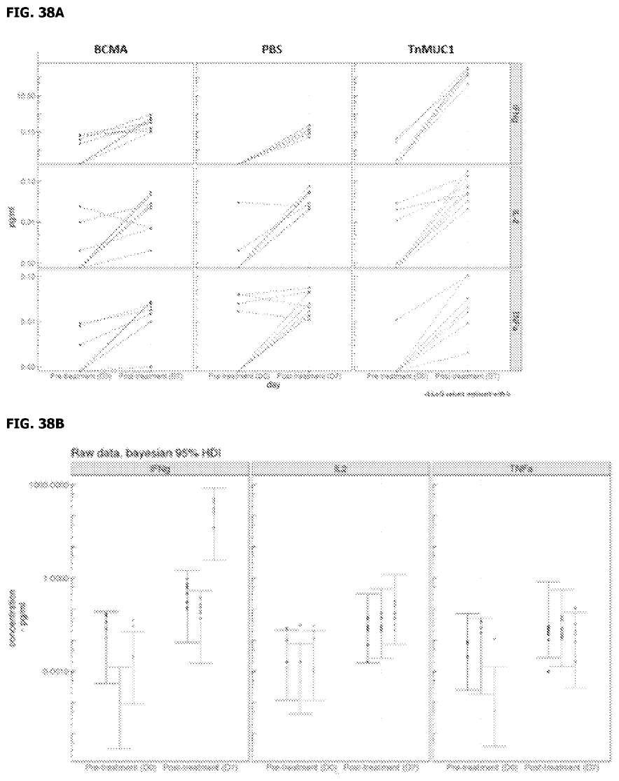

To confirm functional response of MB053 CAR-T cells to the Tn-MUC1 positive tumours, levels of Th1 cytokines (IFN-γ, IL-2 and TNF-α) were measured in the serum. Th1 cytokines secretion by CAR T cells indicates engagement with the target and functional activation of CAR T cells. It was found that CDX tumour-bearing mice dosed with MB053 CAR-T cells had higher levels for all three cytokines when compared to pre-treatment cytokines levels on Day 0 (FIG. 38A). Levels of IFN-γ were significantly higher in serum of the MB053 CAR-T cell group compared to cytokines levels in serum of PBS group (FIG. 38B). A trend of increased secretion of IL-2 was observed in the MB053 CAR-T cell group compared to PBS and BCMA groups post-treatment; however the secreted levels were rather low and not biologically relevant. Overall, among all cytokines, IFN-γ secreted levels showed the most prominent increase in the MB053 CAR-T cells group post-treatment.

These results demonstrate that tumour volume reduction results were supported by elevated serum levels of IFN-γ and TNF-α at day 7 post-T cell dosing in mice injected with MB053 CAR T cells. Elevated serum levels of IFN-γ and TNF-α suggest successful engagement of MB053 CAR T cells with the target and subsequent activation resulting in cytokines secretion by CAR T cells. Therefore, MB053 CAR T cells demonstrated high cytotoxic efficacy against CDX tumour implanted in NSG mice.

### Distribution of MB053 CAR-T Cells in Tumours and Murine Tissues by CD3 IHC

In the tumour site of CDX tumour-bearing mice treated with MB053 CAR-T cells, there were mild numbers of human T lymphocytes. They were typically scattered in an apparently random manner throughout the site. Human T lymphocytes were not present in the tumour of any CDX tumour-bearing mouse treated dosed with PBS. It is also worth mentioning that there was no evidence of toxicity related to MB053 CAR-T cells in murine tissues as assessed by histopathology. In addition, there were also no noteworthy microscopic changes in the PBS and BCMA groups.

### Example 18: Assessment of Tn-MUC1 CAR T in Response to Low Concentrations of Tn-MUC1 Positive Tumour Cells

The aim of this study was to measure MB053 CAR T cell functional activity in response to low levels of Tn-MUC1 positive cells through assessment of interferon-gamma (IFNγ) secretion in supernatant after 24h co-culture with Tn-MUC1 positive and negative isogenic cell lines.

### Methods

### Experimental Preparations

### CAR T Cell Generation

TnMUC1 CAR T cells and UT T cells from 3 donors were expanded, checked for transduction efficiency (TE) and frozen.

### Tumour Cell Line Preparation

PC-3 5F5 and Jurkat 6E have more than 90% of Tn-MUC1 positive cells and were used as positive controls (100% Tn-MUC1). PC-3 WT and Jurkat COSMC KI have less than 1% Tn-MUC1 positive cells and were used as negative controls. The isogenic PC-3 cell lines (PC-3 5F5 and PC-3 WT) were diluted to 3×10⁵ cells/mL and the isogenic Jurkat cell lines (Jurkat 6E and Jurkat COSMC KI) were diluted to 5×10⁵ cells/mL.

A population of 20% Tn-MUC1 positivity was generated to a total volume of 10 mL comprised of 20% positive Tn-MUC1 cells mixed with 80% Tn-MUC1 negative cells. A subsequent four-point serial 1 in 2 dilution of the 20% Tn MUC-1 positive cell population was performed to obtain 5 mL of 10%, 5%, 2.5% and 1.25% Tn-MUC1 positive conditions. All generated cell lines were then checked for Tn-MUC1 expression on the surface by flow cytometry.

### Experimental Protocols

### Protocol for Flow Cytometry of T Cells

T cells (2×10⁵ cells per condition) were incubated with 50 μL anti-Fab (10.4 μg/mL) for 45 min at 4° C. Cells were washed twice with FACS buffer (DPBS, 2% FBS, 0.05% sodium azide). T cells were subsequently incubated with 50 μL strep-PE secondary antibody (1 μg/mL) for 45 min at 4° C. Cells were washed twice with FACS buffer (DPBS, 2% FBS, 0.05% sodium azide). Cells were resuspended with 100 μL DAPI viability dye (1 μg/mL). Data was acquired on an MACSQuant Analyser flow cytometer. Analysis was performed using FlowJo, using unstained controls for gating. Daily QC of cytometer performance was evaluated using MACSQuant Calibration beads.

### Protocol for Co-Culture Set Up

100 μL of 5×10⁴ Jurkat cells per well, and 3×10⁴ PC-3 cells per well, were plated into 96 well tissue culture plates. 100 μL of cell culture media was added to all remaining wells of the plate. The culture plates were incubated in a humidified incubator at 37° C., 5% CO₂ for 24 hours prior to T cell addition.

MB053 CAR T cells and UT T cells were added to the plated target cells at 1:1 CAR-T to target ratio, where effector number was based on a 50% transduction efficiency of MB053 CAR T cells. Number of UT T cells was equal to total number of added MB053 CAR T cells. 100 μL of the MB053 CAR T cells, UT T cells or media was added to the 96 well cell culture plate. The culture plates were incubated in a humidified incubator at 37° C., 5% CO₂ for 24 hours prior IFNγ detection.

### Protocol for MSD Assay (IFNγ Detection)

50 μL of supernatant was harvested from plates containing co-culture and transferred to 96 well V-bottom polypropylene plates, centrifuged at 300 g, for 5 min. Supernatants were diluted 1 in 30 in diluent 2 (MSD). The IFNγ calibrator was prepared in Diluent 2 to generate a stock 1230 μg/mL, and was left to equilibrate at room RT for 30 min. 200 μL of calibrator was transferred to a 96 well polypropylene V-bottom plate. A subsequent 8 point 1 in 4 serial dilution was conducted in Diluent 2.

The MSD assay plates were washed 3 times with 150 μL of wash buffer (PBS, 0.05% Tween) and left to dry for 30 min. 25 μL of the diluted supernatants and calibrant dose response curves were transferred to the respective MSD plates. The plates were sealed and incubated at RT for 2 hrs on a plate shaker, 600 rounds per minute (rpm). Following incubation, plates were washed 3 times with 150 μL of wash buffer. Detection antibody was diluted in diluent 3 (MSD) and 25 μL of the antibody was transferred to the MSD assay plates. The plates were sealed and incubated at RT for 2 hrs on a plate shaker at 600 rpm. Following incubation, the plates were washed 3 times. 150 μL of 2×read buffer was added to each well before reading on the MSD Sector 600 Imager. Data was analysed using the MSD Discovery Workbench® software used to extrapolate the concentration of the IFNγ.

### Results and Discussion

### Percentage of Tn-MUC1 Positive Cells Defines Level of IFNγ Secretion

Decreasing the ratio of Tn-MUC1 positive cells was achieved by diluting the Tn-MUC1 positive cell lines, Jurkat 6E or PC-3 5F5, with Tn-MUC1 negative cell lines, Jurkat COSMC KI or PC-3 WT, respectively. As result, a series of mixed isogenic cell lines containing different levels of Tn-MUC1 positive cells was achieved in Jurkat cells (Tn-MUC1 positive cells: 82.7%, 19.3%, 9.8%, 4.8%, 2.4%, 1.4%, 0.05%) and in PC-3 cells (Tn-MUC1 positive cells: 93%, 15%, 8%, 4%, 2.4%, 1.6%, 0.77%) (FIG. 2). A small percentage of Tn-MUC1 positive cells were present in the PC-3 WT cell line (0.77%) and the Jurkat COSMC KI cell line (0.05%).

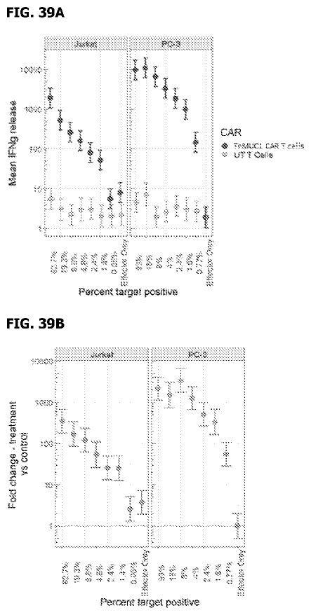

Co-culture of MB053 CAR T cells with a series of mixed isogenic cell lines demonstrated a Tn-MUC1 positivity dependent secretion of IFNγ. In contrast, no apparent IFNγ secretion was detected when these mixed isogenic cell lines were co-cultured with UT T cells (FIG. 39A).

The concentrations of IFNγ produced T cells was converted into a fold increase response. In short, the amount of IFNγ produced by MB053 CAR T cells was divided by the amount of IFNγ produced by its respective UT T cell control, for each mixed cell population co-culture. A fold increase in IFNγ was compared to the lowest percentage Tn-MUC1 positive condition in Jurkat cells (0.05% Tn-MUC1 positive cells) and in PC-3 cells (0.77% Tn-MUC1 positive cells). For the Jurkat derived cell populations there was no significant increase between the lowest Tn-MUC1 positive cell population when compared to MB053 CAR T cells cultured alone (effector only). In contrast, for the PC-3 derived cell populations a significant increase was seen between the lowest Tn-MUC1 positive cell population when compared to the effector only response. All subsequent increases in Tn-MUC1 positivity conferred significant increase in IFNγ levels for all cell populations derived from both Jurkat and PC-3.

Data in this study confirmed that MB053 CAR-T cells can recognise and exert a functional response, which is defined by IFNγ secretion in the presence of very low levels of Tn-MUC1 positive cells. MB053 CAR-T cells demonstrated that a dose dependent response of IFNγ secretion was associated with the level of Tn-MUC1 positivity, which correlated with the number of Tn-MUC1 positive cells in the mixed isogenic cell populations. Co-culture of MB053 CAR-T cells with mixed cell populations which have a low number of Tn-MUC1 positive cells (1.4% Jurkat; 1.6% PC-3) leads to a statistically significant increase (FIG. 39B) in IFNγ release when compared to that seen by UT T cells co-cultured with the same mixed cell populations. While MB053 CAR T cells did not show any activation in response to the Tn-MUC1 negative Jurkat KI cell line, a statistically significant increase in IFNγ release was seen by MB053 CAR T cells in co-culture with the PC-3 cell line which only had 0.77% Tn-MUC1 positive cells.

SEQUENCE LISTING

SEQ ID NO: 1: Amino acid sequence of MB004 scFv
MALPVTALLLPLALLLHAARPELVMTQSPSSLTVTAGEKVTMICKSSQSLLNSGDQKNYLTWYQQKPG
QPPKLLIFWASTRESGVPDRFTGSGSGTDFTLTISSVQAEDLAVYYCQNDYSYPLTFGAGTKLELKGG
GGSGGGGSGGGGSQVQLQQSDAELVKPGSSVKISCKASGYTFTDHAIHWVKQKPEQGLEWIGHFSPG
NTDIKYNDKFKGKATLTVDRSSSTAYMQLNSLTSEDSAVYFCKTSTFFFDYWGQGTTLTVSS
SEQ ID NO: 2: Amino acid sequence of MB021 scFv with signal peptide
MALPVTALLLPLALLLHAARPDIVMTQSPDSLAVSLGERATINCKSSQSLLNSGDQKNYLTWYQQKPG
QPPKLLIYWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWVRQAPGQGLEWM
GHFSPGNTDIKYNDKFKGRVTITADKSTSTAYMELSSLRSEDTAVYYCKTSTFFFDYWGQGTTVTVSS
SEQ ID NO: 3: Amino acid sequence of MB022 scFv with signal peptide
MALPVTALLLPLALLLHAARPDIVMTQSPDSLAVSLGERATINCKSSQSLLNSGDQKNYLTWYQQKPG
QPPKLLIYWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWVRQAPGQGLEWIG
HFSPGNTDIKYNDKFKGRATLTVDRSTSTAYMELSSLRSEDTAVYFCKTSTFFFDYWGQGTTVTVSS
SEQ ID NO: 4: Amino acid sequence of MB023 scFv with signal peptide
MALPVTALLLPLALLLHAARPELVMTQSPDSLAVSLGERATINCKSSQSLLNSGDQKNYLTWYQQKPG
QPPKLLIYWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWVRQAPGQGLEWI
GHFSPGNTDIKYNDKFKGRATLTVDRSTSTAYMELSSLRSEDTAVYFCKTSTFFFDYWGQGTTVTV
SS
SEQ ID NO: 5: Amino acid sequence of MB024 scFv with signal peptide
MALPVTALLLPLALLLHAARPDIVMTQSPDSLAVSLGERATINCKSSQSLLNSGDQKNYLTWYQQKPG
QPPKLLIFWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWRQAPGQGLEWM
GHFSPGNTDIKYNDKFKGRVTITADKSTSTAYMELSSLRSEDTAVYYCKTSTFFFDYWGQGTTVTVS
S
SEQ ID NO: 6: Amino acid sequence of MB025 scFv with signal peptide
MALPVTALLLPLALLLHAARPDIVMTQSPDSLAVSLGERATINCKSSQSLLNSGDQKNYLTWYQQKPG
QPPKLLIFWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWVRQAPGQGLEWI
GHFSPGNTDIKYNDKFKGRATLTVDRSTSTAYMELSSLRSEDTAVYFCKTSTFFFDYWGQGTTVTV
SS
SEQ ID NO: 7: Amino acid sequence of MB026 scFv with signal peptide
MALPVTALLLPLALLLHAARPELVMTQSPDSLAVSLGERATINCKSSQSLLNSGDQKNYLTWYQQKPG
QPPKLLIFWASTRESGVPDRFSGSGSGTDFTLTISSLQAEDVAVYYCQNDYSYPLTFGQGTKLEIKGG
GGSGGGGSGGGGSGGGGSQVQLVQSGAEVKKPGSSVKVSCKASGYTFTDHAIHWRQAPGQGLEWI
GHFSPGNTDIKYNDKFKGRATLTVDRSTSTAYMELSSLRSEDTAVYFCKTSTFFFDYWGQGTTVTV
SS
SEQ ID NO: 8: Amino acid sequence of CD8a signal peptide
MALPVTALLLPLALLLHAARP
SEQ ID NO: 9: Amino acid sequence of GGGGS linker
GGGGSGGGGSGGGGS
SEQ ID NO: 10: Amino acid sequence of MB021 CDRL1
KSSQSLLNSGDQKNYLT
SEQ ID NO: 11: Amino acid sequence of MB021 CDRL2
WASTRES
SEQ ID NO: 12: Amino acid sequence of MB021 CDRL3
QNDYSYPLT
SEQ ID NO: 13: Amino acid sequence of MB021 CDRH1
DHAIH
SEQ ID NO: 14: Amino acid sequence of MB021 CDRH2
HFSPGNTDIKYNDKFKG
SEQ ID NO: 15: Amino acid sequence of MB021 CDRH3
STFFFDY
SEQ ID NO: 16: Amino acid sequence of MB022 CDRL1
KSSQSLLNSGDQKNYLT
SEQ ID NO: 17: Amino acid sequence of MB022 CDRL2
WASTRES
SEQ ID NO: 18: Amino acid sequence of MB022 CDRL3
QNDYSYPLT
SEQ ID NO: 19: Amino acid sequence of MB022 CDRH1
DHAIH
SEQ ID NO: 20: Amino acid sequence of MB022 CDRH2
HFSPGNTDIKYNDKFKG
SEQ ID NO: 21: Amino acid sequence of MB022 CDRH3
STFFFDY
SEQ ID NO: 22: Amino acid sequence of MB023 CDRL1
KSSQSLLNSGDQKNYLT
SEQ ID NO: 23: Amino acid sequence of MB023 CDRL2
WASTRES
SEQ ID NO: 24: Amino acid sequence of MB023 CDRL3
QNDYSYPLT
SEQ ID NO: 25: Amino acid sequence of MB023 CDRH1
DHAIH
SEQ ID NO: 26: Amino acid sequence of MB023 CDRH2
HFSPGNTDIKYNDKFKG
SEQ ID NO: 27: Amino acid sequence of MB023 CDRH3
STFFFDY
SEQ ID NO: 28: Amino acid sequence of MB024 CDRL1
KSSQSLLNSGDQKNYLT
SEQ ID NO: 29: Amino acid sequence of MB024 CDRL2
WASTRES
SEQ ID NO: 30: Amino acid sequence of MB024 CDRL3
QNDYSYPLT
SEQ ID NO: 31: Amino acid sequence of MB024 CDRH1
DHAIH
SEQ ID NO: 32: Amino acid sequence of MB024 CDRH2
HFSPGNTDIKYNDKFKG
SEQ ID NO: 33: Amino acid sequence of MB024 CDRH3
STFFFDY
SEQ ID NO: 34: Amino acid sequence of MB025 CDRL1
KSSQSLLNSGDQKNYLT
SEQ ID NO: 35: Amino acid sequence of MB025 CDRL2
WASTRES
SEQ ID NO: 36: Amino acid sequence of MB025 CDRL3
QNDYSYPLT
SEQ ID NO: 37: Amino acid sequence of MB025 CDRH1
DHAIH
SEQ ID NO: 38: Amino acid sequence of MB025 CDRH2
HFSPGNTDIKYNDKFKG
SEQ ID NO: 39: Amino acid sequence of MB025 CDRH3
STFFFDY
SEQ ID NO: 40: Amino acid sequence of MB026 CDRL1
KSSQSLLNSGDQKNYLT
SEQ ID NO: 41: Amino acid sequence of MB026 CDRL2
WASTRES
SEQ ID NO: 42: Amino acid sequence of MB026 CDRL3
QNDYSYPLT
SEQ ID NO: 43: Amino acid sequence of MB026 CDRH1
DHAIH
SEQ ID NO: 44: Amino acid sequence of MB026 CDRH2
HFSPGNTDIKYNDKFKG
SEQ ID NO: 45: Amino acid sequence of MB026 CDRH3
STFFFDY
SEQ ID NO: 46: Amino acid sequence of MB021 VL
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIK
SEQ ID NO: 47: Amino acid sequence of MB021 VH
QVQLVQSGAEVKKPGSSVKVSCKASGYTFTDHAIHWVRQAPGQGLEWMGHFSPGNTDIKYNDKFKGRV
TITADKSTSTAYMELSSLRSEDTAVYYCKTSTFFFDYWGQGTTVTVSS
SEQ ID NO: 48: Amino acid sequence of MB022 VL
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIK
SEQ ID NO: 49: Amino acid sequence of MB022 VH
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYFCKTSTFFFDYWGQGTTVTVSS
SEQ ID NO: 50: Amino acid sequence of MB023 VL
ELVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIK
SEQ ID NO: 51: Amino acid sequence of MB023 VH
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYFCKTSTFFFDYWGQGTTVTVSS
SEQ ID NO: 52: Amino acid sequence of MB024 VL
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIK
SEQ ID NO: 53: Amino acid sequence of MB024 VH
QVQLVQSGAEVKKPGSSVKVSCKASGYTFTDHAIHWVRQAPGQGLEWMGHFSPGNTDIKYNDKFKGRV
TITADKSTSTAYMELSSLRSEDTAVYYCKTSTFFFDYWGQGTTVTVSS
SEQ ID NO: 54: Amino acid sequence of MB025 VL
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIK
SEQ ID NO: 55: Amino acid sequence of MB025 VH
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYFCKTSTFFFDYWGQGTTVTVSS
SEQ ID NO: 56: Amino acid sequence of MB026 VL
ELVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIK
SEQ ID NO: 57: Amino acid sequence of MB026 VH
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYFCKTSTFFFDYWGQGTTVTVSS
SEQ ID NO: 58: Amino acid sequence of MB051 CAR
MALPVTALLLPLALLLHAARPDIVMTQSPDSLAVSLGERATINCKSSQSLLNSGDQKNYLTWYQQKPGQP
PKLLIYWASTRESGVPDRFSGSGSGTDFTLTISSLQAEDVAVYYCQNDYSYPLTFGQGTKLEIKGGGGSG
GGGSGGGGSGGGGSQVQLVQSGAEVKKPGSSVKVSCKASGYTFTDHAIHWVRQAPGQGLEWMGHFSP
GNTDIKYNDKFKGRVTITADKSTSTAYMELSSLRSEDTAVYYCKTSTFFFDYWGQGTTVTVSSRTFVPVF
LPAKPTTTPAPRPPTPAPTIASQPLSLRPEACRPAAGGAVHTRGLDFACDIYIWAPLAGTCGVLLLSLVITL
YCNHRNKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 59: Amino acid sequence of MB052 CAR
MALPVTALLLPLALLLHAARPDIVMTQSPDSLAVSLGERATINCKSSQSLLNSGDQKNYLTWYQQKPGQP
PKLLIYWASTRESGVPDRFSGSGSGTDFTLTISSLQAEDVAVYYCQNDYSYPLTFGQGTKLEIKGGGGSG
GGGSGGGGSGGGGSQVQLVQSGAEVKKPGSSVKVSCKASGYTFTDHAIHWVRQAPGQGLEWIGHFSP
GNTDIKYNDKFKGRATLTVDRSTSTAYMELSSLRSEDTAVYFCKTSTFFFDYWGQGTTVTVSSRTFVPVF
LPAKPTTTPAPRPPTPAPTIASQPLSLRPEACRPAAGGAVHTRGLDFACDIYIWAPLAGTCGVLLLSLVITL
YCNHRNKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 60: Amino acid sequence of MB053 CAR
MALPVTALLLPLALLLHAARPDIVMTQSPDSLAVSLGERATINCKSSQSLLNSGDQKNYLTWYQQKPGQP
PKLLIFWASTRESGVPDRFSGSGSGTDFTLTISSLQAEDVAVYYCQNDYSYPLTFGQGTKLEIKGGGGSG
GGGSGGGGSGGGGSQVQLVQSGAEVKKPGSSVKVSCKASGYTFTDHAIHWVRQAPGQGLEWMGHFSP
GNTDIKYNDKFKGRVTITADKSTSTAYMELSSLRSEDTAVYYCKTSTFFFDYWGQGTTVTVSSRTFVPVF
LPAKPTTTPAPRPPTPAPTIASQPLSLRPEACRPAAGGAVHTRGLDFACDIYIWAPLAGTCGVLLLSLVITL
YCNHRNKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 61: Amino acid sequence of MB054 CAR
MALPVTALLLPLALLLHAARPDIVMTQSPDSLAVSLGERATINCKSSQSLLNSGDQKNYLTWYQQKPGQP
PKLLIFWASTRESGVPDRFSGSGSGTDFTLTISSLQAEDVAVYYCQNDYSYPLTFGQGTKLEIKGGGGSG
GGGSGGGGSGGGGSQVQLVQSGAEVKKPGSSVKVSCKASGYTFTDHAIHWVRQAPGQGLEWIGHFSP
GNTDIKYNDKFKGRATLTVDRSTSTAYMELSSLRSEDTAVYFCKTSTFFFDYWGQGTTVTVSSRTFVPVF
LPAKPTTTPAPRPPTPAPTIASQPLSLRPEACRPAAGGAVHTRGLDFACDIYIWAPLAGTCGVLLLSLVITL
YCNHRNKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 62: VL domain from murine mAb 5e5
ELVMTQSPSSLTVTAGEKVTMICKSSQSLLNSGDQKNYLTWYQQKPGQPPKLLIFWASTRESGVPDRFT
GSGSGTDFTLTISSVQAEDLAVYYCQNDYSYPLTFGAGTKLELK
SEQ ID NO: 63: VH domain from murine mAb 5e5
QVQLQQSDAELVKPGSSVKISCKASGYTFTDHAIHWVKQKPEQGLEWIGHFSPGNTDIKYNDKFKGKAT
LTVDRSSSTAYMQLNSLTSEDSAVYFCKTSTFFFDYWGQGTTLTVSS
SEQ ID NO: 64: VL domain from murine mAb 5e5 with masked CDRs
ELVMTQSPSSLTVTAGEKVTMICXXXXXXXXXXXXXXXXXWYQQKPGQPPKLLIFXXXXXXXGVPDRFTG
SGSGTDFTLTISSVQAEDLAVYYCXXXXXXXXXFGAGTKLELK
SEQ ID NO: 65: VH domain from murine mAb 5e5 with masked CDRs
QVQLQQSDAELVKPGSSVKISCKASGYTFTXXXXXWVKQKPEQGLEWIGXXXXXXXXXXXXXXXXXKATL
TVDRSSSTAYMQLNSLTSEDSAVYFCKTXXXXXXXWGQGTTLTVSS
SEQ ID NO: 66: Humanised VH chain H0

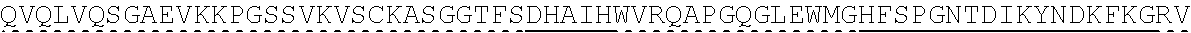

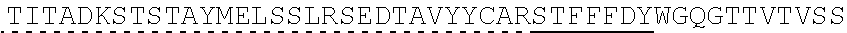

SEQ ID NO: 67: Humanised VH chain H1
QVQLVQSGAEVKKPGSSVKVSCKASGYTFTDHAIHWVRQAPGQGLEWMGHFSPGNTDIKYNDKFKGRV
TITADKSTSTAYMELSSLRSEDTAVYYCARSTFFFDYWGQGTTVTVSS
SEQ ID NO: 68: Humanised VH chain H2
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYYCARSTFFFDYWGQGTTVTVSS
SEQ ID NO: 69: Humanised VH chain H3
QVQLVQSGAEVKKPGSSVKVSCKASGYTFTDHAIHWVRQAPGQGLEWMGHFSPGNTDIKYNDKFKGRV
TITADKSTSTAYMELSSLRSEDTAVYYCKTSTFFFDYWGQGTTVTVSS
SEQ ID NO: 70: Humanised VH chain H4
QVQLVQSGAEVKKPGSSVKVSCKASGYTFTDHAIHWVRQAPGQGLEWMGHFSPGNTDIKYNDKFKGRV
TITADKSTSTAYMELSSLRSEDTAVYFCARSTFFFDYWGQGTTVTVSS
SEQ ID NO: 71: Humanised VH chain H5
QVQLVQSGAEVKKPGSSVKVSCKASGYTFTDHAIHWVRQAPGQGLEWIGHFSPGNTDIKYNDKFKGRA
TLTVDRSTSTAYMELSSLRSEDTAVYFCKTSTFFFDYWGQGTTVTVSS
SEQ ID NO: 72: Humanised VL chain L0
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIK
SEQ ID NO: 73: Humanised VL chain L1
ELVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIK
SEQ ID NO: 74: Humanised VL chain L2
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIK
SEQ ID NO: 75: Humanised VL chain L3
ELVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIK
SEQ ID NO: 76: Tn-MUC1 peptide (fully glycosylated)
Biotin-PEG2-GV-T(AcNH-a-Gal)-S(AcNH-a-Gal)-APD-T(AcNH-a-Gal)-RPAPGS(AcNH-a-Gal)-
T(AcNH-a-Gal)-APPAH-amide
SEQ ID NO: 77: MUC1 peptide (non-glycosylated)
Biotin-[PEG2]-GVTSAPDTRPAPGSTAPPAH-amide
SEQ ID NO: 78: humanised anti-TnMUC1 scFv construct 88A (including hexa-His tag)
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKVEIKGGGGSGGGGSGGGGSGGGGSQVQL
VQSGAEVKKTGSSVKVSCKASGYTFTDHAIHWVRQAPGQALEWMGHFSPGNTDIKYNDKFKGRVTLTV
DRSMSTAYMELSSLRSEDTAMYYCKTSTFFFDYWGQGTMVTVSSAAAHHHHHH
SEQ ID NO: 79: humanised anti-TnMUC1 scFv construct 82A (including hexa-His tag)
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWMGHFSPGNTDIKYNDKFKGRVTITAD
KSTSTAYMELSSLRSEDTAVYYCKTSTFFFDYWGQGTTVTVSSAAAHHHHHH
SEQ ID NO: 80: humanised anti-TnMUC1 scFv construct 89A (including hexa-His tag)
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWIGHFSPGNTDIKYNDKFKGRATLTVDR
STSTAYMELSSLRSEDTAVYFCKTSTFFFDYWGQGTTVTVSSAAAHHHHHH
SEQ ID NO: 81: humanised anti-TnMUC1 scFv construct 94A (including hexa-His tag)
ELVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWIGHFSPGNTDIKYNDKFKGRATLTVDR
STSTAYMELSSLRSEDTAVYFCKTSTFFFDYWGQGTTVTVSSAAAHHHHHH
SEQ ID NO: 82: humanised anti-TnMUC1 scFv construct 97A (including hexa-His tag)
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWMGHFSPGNTDIKYNDKFKGRVTITADKS
TSTAYMELSSLRSEDTAVYYCKTSTFFFDYWGQGTTVTVSSAAAHHHHHH
SEQ ID NO: 83: humanised anti-TnMUC1 scFv construct 19A (including hexa-His tag)
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWIGHFSPGNTDIKYNDKFKGRATLTVDRS
TSTAYMELSSLRSEDTAVYFCKTSTFFFDYWGQGTTVTVSSAAAHHHHHH
SEQ ID NO: 84: humanised anti-TnMUC1 scFv construct 77A (including hexa-His tag)
ELVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWIGHFSPGNTDIKYNDKFKGRATLTVDRS
TSTAYMELSSLRSEDTAVYFCKTSTFFFDYWGQGTTVTVSSAAAHHHHHH
SEQ ID NO: 85: TnMUC1 peptide 1 (partially glycosylated)
Biotin-PEG2-GV-TSAPD-TRPAPGS(AcNH-a-Gal)-T(AcNH-a-Gal)-APPAH-amide
SEQ ID NO: 86: TnMUC1 peptide 2 (partially glycosylated)
Biotin-PEG2-GV-T(AcNH-a-Gal)-S(AcNH-a-Gal)-APD-T(AcNH-a-Gal)-RPAPGSTAPPAH-amide
SEQ ID NO: 87: STnMUC1 peptide
Biotin-[PEG]2-GVTSAPDTRPAPG-[Ser(Sial-AcNH-a-Gal)]-[Thr(Sial-AcNH-a-Gal)]-APPAH-amide
SEQ ID NO: 88: TnMUC1 peptide
GV-T(AcNH-a-Gal)-S(AcNH-a-Gal)-APD-T(AcNH-a-Gal)-RPAPGS(AcNH-a-Gal)-T(AcNH-a-Gal)-
APPAH-amide
SEQ ID NO: 89: MUC1 peptide
GVTSAPDTRPAPGSTAPPAH-amide
SEQ ID NO: 90: Amino acid sequence of MB021 scFv
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWMGHFSPGNTDIKYNDKFKGRVTITAD
KSTSTAYMELSSLRSEDTAVYYCKTSTFFFDYWGQGTTVTVSS
SEQ ID NO: 91: Amino acid sequence of MB022 scFv
DIVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWIGHFSPGNTDIKYNDKFKGRATLTVDR
STSTAYMELSSLRSEDTAVYFCKTSTFFFDYWGQGTTVTVSS
SEQ ID NO: 92: Amino acid sequence of MB023 scFv
ELVMTQSPDSLAVSLGERATINCKSSQSLLNSGDQKNYLTWYQQKPGQPPKLLIYWASTRESGVPDRFS
GSGSGTDFTLTISSLQAEDVAVYYCQNDYSYPLTFGQGTKLEIKGGGGSGGGGSGGGGSGGGGSQVQLV
QSGAEVKKPGSSVKVSCKASGYTFTDHAIHWVRQAPGQGLEWIGHFSPGNTDIKYNDKFKGRATLTVDR
STSTAYMELSSLRSEDTAVYFCKTSTFFFDYWGQGTTVTVSS
SEQ ID NO: 93: Amino acid sequence of MB024 scFv
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWMGHFSPGNTDIKYNDKFKGRVTITADKS
TSTAYMELSSLRSEDTAVYYCKTSTFFFDYWGQGTTVTVSS
SEQ ID NO: 94: Amino acid sequence of MB025 scFv
DIVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWIGHFSPGNTDIKYNDKFKGRATLTVDRS
TSTAYMELSSLRSEDTAVYFCKTSTFFFDYWGQGTTVTVSS
SEQ ID NO: 95: Amino acid sequence of MB026 scFv
ELVMTQSPDSLAVSLGERATINCKSSQSLLNSGDQKNYLTWYQQKPGQPPKLLIFWASTRESGVPDRFSG
SGSGTDFTLTISSLQAEDVAVYYCQNDYSYPLTFGQGTKLEIKGGGGSGGGGSGGGGSGGGGSQVQLVQ
SGAEVKKPGSSVKVSCKASGYTFTDHAIHWVRQAPGQGLEWIGHFSPGNTDIKYNDKFKGRATLTVDRS
TSTAYMELSSLRSEDTAVYFCKTSTFFFDYWGQGTTVTVSS
SEQ ID NO: 96: TnMUC1 peptide
GV-T(AcNH-a-Gal)-S(AcNH-a-Gal)-APD-T(AcNH-a-Gal)-RPAPGS(AcNH-a-Gal)-T(AcNH-a-Gal)-
APPAH
SEQ ID NO: 97: MUC1 peptide
GVTSAPDTRPAPGSTAPPAH
SEQ ID NO: 98: TnMUC1 peptide 1
GVTSAPDTRPAPGS(AcNH-a-Gal)T(AcNH-a-Gal)APPAH
SEQ ID NO: 99: TnMUC1 peptide 2
GVT(AcNH-a-Gal)S(AcNH-a-Gal)APDT(AcNH-a-Gal)RPAPGSTAPPAH
SEQ ID NO: 100: STnMUC1 peptide
GVTSAPDTRPAPG-[Ser(Sial-AcNH-a-Gal)]-[Thr(Sial-AcNH-a-Gal)]-APPAH

1. A chimeric antigen receptor (CAR) comprising:
a) an extracellular domain which comprises a humanised antibody or antigen binding domain thereof that binds one or more epitopes on an aberrantly glycosylated MUCI protein, wherein the antibody of antigen binding domain thereof comprises:
a CDRL1 sequence at least 90% identical to SEQ ID NO: 28;
a CDRL2 sequence at least 90% identical to SEQ ID NO: 29;
a CDRL3 sequence at least 90% identical to SEQ ID NO: 30;
a CDRH1 sequence at least 90% identical to SEQ ID NO: 31;
a CDRH2 sequence at least 90% identical to SEQ ID NO: 32; and
a CDRH3 sequence at least 90% identical to SEQ ID NO: 33,
and wherein the antibody or antigen binding fragment thereof binds said epitope with a faster dissociation rate constant (k_{d}) as compared to a non-humanised version of said antibody or antigen binding domain thereof, and
b) a transmembrane domain;
c) one or more costimulatory domains; and
d) one or more intracellular signalling domains.

2. A CAR according to claim 1, wherein the antibody or antigen binding domain thereof comprises:
a CDRL1 sequence of SEQ ID NO: 28;
a CDRL2 sequence of SEQ ID NO: 29;
a CDRL3 sequence of SEQ ID NO: 30;
a CDRH1 sequence of SEQ ID NO: 31;
a CDRH2 sequence of SEQ ID NO: 32; and
a CDRH3 sequence of SEQ ID NO: 33.

3. A CAR according to claim 1, wherein the antibody or antigen binding domain thereof is selected from the group consisting of: a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

4. A CAR according to claim 3, wherein the antibody or antigen binding domain thereof is a scFv.

5. A CAR according to claim 1, wherein the antibody or antigen binding domain thereof comprises a variable light chain sequence at least 90% identical to a sequence selected from the group consisting of SEQ ID NOs: 46, 48, 50, 52, 54, and 56, and a variable heavy chain sequence at least 90% identical to a sequence selected from a group consisting of SEQ ID NOS: 47, 49, 51, 53, 55, and 57.

6. A CAR according to claim 5, wherein the antibody or antigen binding domain thereof comprises a variable light chain sequence selected from the group consisting of SEQ ID NOs: 46, 48, 50, 52, 54, and 56, and a variable heavy chain sequence selected from the group consisting of SEQ ID NOs: 47, 49, 51, 53, 55, and 57.

7. A CAR according to claim 1, wherein the aberrantly glycosylated MUC1 protein is either TnMUC1 or STnMUC1.

8. A CAR according to claim 1, wherein the transmembrane domain is isolated from a polypeptide selected from the group consisting of: alpha or beta chain of the T-cell receptor, CD3δ, CD3ε, CD3γ, CD3ζ, CD4, CD5, CD8α CD9, CD16, CD22, CD27, CD28, CD33, CD37, CD45, CD64, CD80, CD86, CD134, CD137 (4-1BB), CD152, CD154, CD278 (ICOS), and PD1.

9. A CAR according to claim 8, wherein the transmembrane domain is isolated from CD8α.

10. A CAR according to claim 1, wherein the one or more costimulatory domains is isolated from a costimulatory molecule selected from the group consisting of: CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TLR1, TLR2, TLR3, TLR4, TLR5, TLR6, TLR7, TLR8, TLR9, TLR10, TRIM, and ZAP70.

11. A CAR according to claim 10, wherein the one or more costimulatory domains is isolated from CD137 (4-1BB).

12. A CAR according to claim 1, wherein the one or more intracellular signalling domains is isolated from an intracellular signalling molecule selected from the group consisting of: FcRγ, FcRβ, CD3γ, CD3ε, CD3δ, CD3ζ, CD22, CD66d, CD79a, and CD79b.

13. A CAR according to claim 12, wherein the one or more intracellular signalling domains is isolated from CD3ζ.

14. A CAR according to claim 1 comprising a sequence at least 90% identical to a sequence selected from the group consisting of SEQ ID NOs: 90, 91, 92, 93, 94, and 95.

15. A CAR according to claim 1 comprising a sequence selected from the group consisting of SEQ ID NOs: 90, 91, 92, 93, 94, and 95.

16. A CAR according to claim 1 comprising a sequence at least 90% identical to a sequence selected from the group consisting of SEQ ID NOs: 58, 59, 60, and 61.

17. A CAR according to claim 1 comprising a sequence selected from the group consisting of SEQ ID NOs: 58, 59, 60, and 61.

18. A polypeptide comprising the amino acid sequence of the CAR according to claim 1.

19. A polynucleotide encoding a CAR according to claim 1.

20. A vector comprising the polynucleotide according to claim 19.

21.-25. (canceled)

26. A vector producer cell comprising the vector according to claim 20.

27. An immune effector cell comprising a vector according to claim 20.

28.-29. (canceled)

30. A pharmaceutical composition comprising the immune effector cell according to claim 27 and a pharmaceutically acceptable excipient.

31. A method of generating an immune effector cell comprising a CAR comprising introducing into an immune effector cell a vector according to claim 20.

32.-36. (canceled)

37. A method for the treatment of cancer in a subject in need thereof, comprising administering to the subject a therapeutically effective amount of a CAR according to claim 1, wherein the cancer comprises cells which express an aberrantly glycosylated MUC1 protein.

38. A method for increasing the cytotoxicity in cancer cells that express an aberrantly glycosylated MUC1 protein in a subject having cancer, comprising administering to the subject a CAR according to claim 1, in an amount sufficient to increase the cytotoxicity in cancer cells that express an aberrantly glycosylated MUC1 compared to the cytotoxicity of the cancer cells that express an aberrantly glycosylated MUC1 protein prior to the administration

39. A method for decreasing the number of cancer cells expressing an aberrantly glycosylated MUC1 protein in a subject having cancer, comprising administering to the subject a therapeutically effective amount of a CAR according to claim 1, wherein the therapeutically effective amount is sufficient to decrease the number of cancer cells that express an aberrantly glycosylated MUC1 protein compared to the number of the cancer cells that express an aberrantly glycosylated MUC1 protein prior to the administration.

40.-45. (canceled)
